(19) United States
(12) Patent Application Publication
(10) Pub. No.: US 2025/0388961 A1
(43) Pub. Date: Dec. 25, 2025

(54) METHODS FOR SEQUENCING AN IMMUNE CELL RECEPTOR

(71) Applicant: The Johns Hopkins University, Baltimore, MD (US)

(72) Inventors: Bert Vogelstein, Baltimore, MD (US); Kenneth W. Kinzler, Frankford, DE (US); Nickolas Papadopoulos, Towson, MD (US); Alexander H. Pearlman, Baltimore, MD (US); Joshua David Cohen, Baltimore, MD (US)

(21) Appl. No.: 18/835,650

(22) PCT Filed: Feb. 3, 2023
(86) PCT No.: PCT/US2023/012290

§ 371 Date: Aug. 2, 2024

Related U.S. Application Data
(60) Provisional application No. 63/306,439, filed on Feb. 3, 2022.

(51) Int. Cl. C12Q 1/6869; C12Q 1/6806; C12Q 1/6827; C12Q 1/6855; C12Q 1/686

(52) U.S. Cl. CPC C12Q 1/6869; C12Q 1/6806; C12Q 1/6827; C12Q 1/6855; C12Q 1/686

(57) ABSTRACT

Provided herein are methods for determining a sequence of a double stranded DNA molecule of an immune cell receptor (e.g., T cell receptor, B cell receptor).

### CROSS-REFERENCE TO RELATED APPLICATIONS

This application is a U.S. National Stage application under 35 U.S.C. § 371 and claims the benefit of priority to International Application No. PCT/US2023/012290 having an International Filing date of 3 Feb. 2023, which claims priority to U.S. Provisional Patent Application No. 63/306,439, filed on Feb. 3, 2022, which is incorporated herein by reference in its entirety.

### FEDERALLY SPONSORED RESEARCH OR DEVELOPMENT

This invention was made with government support under grant CA006973, GM008752, and GM136577 awarded by the National Institutes of Health. The government has certain rights in the invention.

### SEQUENCE LISTING

This application contains a Sequence Listing that has been submitted electronically as an XML file named “44807-0406US1_SL_ST26.XML.” The XML file, created on Aug. 9, 2024, is 60,163 bytes in size. The material in the XML file is hereby incorporated by reference in its entirety.

### TECHNICAL FIELD

The present disclosure relates to the area of nucleic acid analysis. In particular, it relates to nucleic acid sequence analysis which can determine a sequence of an immune cell receptor (e.g., B cell receptor, T cell receptor) and detect mutations of the nucleic acid sequence.

### BACKGROUND

B cell (BCR) and T cell (TCR) receptors underly the function of the adaptive immune system. A large and diverse repertoire of BCR and TCR receptors is generated through somatic recombination with imprecise joining of variable (V), diversity (D), and joining (J) genes. Comprehensive characterizations of BCR and TCR repertoires is important for applications including understanding immune responses to pathogens, malignancies, and self-antigens. Tracking specific BCR and TCR sequences is also important for understanding clonal cell dynamics and responses in health and disease. Because individual clones can be rare, methods that enable accurate determination of sequences and precise quantification of sequence abundance are essential.

High throughput sequencing can be used for the characterization of TCR and BCR repertoires. Existing methods for library preparation that begin with RNA as a template generally use adapter ligation or 5′ RACE strategies. These methods can incorporate unique identifiers (UIDs) to increase accuracy. However, because cells can contain multiple BCR or TCR transcripts, quantification of clone abundance is confounded. In addition, RNA templates may not be obtainable from samples with decreased nucleotide quality, including fixed specimens. Methods that begin with DNA as a template for library preparation use multiplex PCR schemes or gene capture schemes. These methods are subject to bias from sources that include primer competition and differential amplification efficiencies. Complex methods are required to account for bias such as computational corrections, the use of spike-in standards, and primer balancing. Accordingly, existing methods for BCR and TCR sequencing are expensive, complex, require sophisticated or elaborate library preparation methods, or exhibit elements of all of these limitations. Moreover, even advanced methods still display systematic biases along with limitations in sensitivity, reproducibility, and quantification accuracy.

Although next generation sequencing methods are, in principle, well suited for the ascertainment and quantification of TCR and BCR sequences, in practice, the error rate of the sequencing itself is too high to allow confident detection of TCR or BCR sequences present at low frequencies in the original sample. One type of strategy to overcome this obstacle involves bioinformatic analysis to calculate probabilities that an observed sequence is more likely to be due to its presence in the original sample rather than to be a technical artifact. But, this strategy alone is often insufficient to detect rare sequences with the high confidence optimal for clinical use, inspiring the use of molecular barcodes to tag every original template molecule. With molecular barcoding, redundant sequencing of the PCR-generated progeny of each tagged molecule is performed and sequencing errors are easily recognized.

Two types of molecular barcodes have been described: exogenous and endogenous. Exogenous barcodes, consisting of pre-specified or random nucleotides, are appended during library preparation or during PCR. Endogenous barcodes are formed by the sequences at the 5′ and 3′ ends of the template fragments. Endogenous barcodes allow “duplex sequencing”, wherein each of the two strands (Watson and Crick) of the original DNA duplex can be discerned by the 5′ to 3′ directionality revealed upon sequencing. Duplex sequencing reduces sequencing errors because it is extremely unlikely that both strands of DNA contain the identical mutation if that mutation was erroneously generated during library preparation or sequencing. A variety of molecular barcoding approaches based on either endogenous or exogenous barcodes, or the combination thereof, have been developed and applied to a wide range of clinical applications.

A barcoding strategy that appends the identical exogenous barcode to the Watson and Crick strands of a template molecule allows unambiguous determination of the identity of the two strands of a template without reference to the endogenous sequence ends. And, because the method involves duplex sequencing, the error rate is minimal. Although this method has the lowest error rate of any sequencing technology described to date, two issues have limited its clinical applicability. First, it is challenging to convert a large fraction of the initial template molecules to adapter-ligated fragments with the same barcode on each strand. This issue is particularly problematic when the amount of initial DNA is limiting, such as found in cell-free plasma DNA used for liquid biopsies. Second, hybridization-based capture is used to enrich for desired regions of the genome. While effective for enriching large regions of interest, hybridization capture is not well suited for TCR or BCR applications, does not scale well for small target regions, and exhibits poor duplex recovery. Sequential rounds of capture can partially overcome these limitations, but existing hybridization capture-based methods typically recover a minority of input molecules with sequence information from both strands. When the targeted region is very small (e.g. one or a few positions in the genome of particular interest), or the amount of DNA available is limited (e.g. <33 ng, as often found in plasma), capture-based approaches are suboptimal. There is therefore a need for methods that can reliably ascertain and quantify TCR and BCR sequences.

### SUMMARY

Provided herein are methods for determining a sequence of a double stranded DNA molecule of an immune cell receptor, the method comprising: (a) attaching a 3′ adapter fragment to each 3′ end of the double-stranded DNA molecule and a 5′ adapter fragment to each 5′ end of the double-stranded DNA molecule to generate an adapted double-stranded DNA molecule, wherein the adapted double-stranded DNA molecule comprises an adapted Watson strand and an adapted Crick strand, wherein the 3′ adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises performing a round of linear extension of the adapted double-stranded DNA molecule, generating an adapted double-stranded Watson template and an adapted double-stranded Crick template; (c) generating a first population of analyte DNA fragments from the adapted double-stranded Watson template and generating a first sequencing read for at least one member of the first population of analyte DNA fragments; (d) generating a second population of analyte DNA fragments from the adapted double-stranded Crick template and generating a second sequencing read for at least one member of the second population of analyte DNA fragments; (e) grouping the first sequencing reads according to the molecular barcode present on the at least one member of the first population of analyte DNA fragments to generate a first analyte DNA family; (f) grouping the second sequencing reads according to the molecular barcode present on the at least one member of the second population of analyte DNA fragments to generate a second analyte DNA family; (g) analyzing the first sequencing read of the first analyte DNA family; and (h) analyzing the second sequencing read of the second analyte DNA family, thus, determining the sequence of the double stranded DNA molecule.

In some embodiments, the 3′ adaptor fragment comprises a partially double-stranded molecular barcode. In some embodiments, the partially double-stranded molecular barcode comprises an endogenous barcode, an exogenous barcode, or both.

In some embodiments, the copying step (b) further comprises performing the round of linear extension of the adapted double-stranded DNA molecule with (i) a first primer complementary to the 3′ adapter sequence, and (ii) a second primer complementary to the complement of the 5′ adapter sequence.

In some embodiments, the generating steps (c) and (d) are performed under PCR conditions. In some embodiments, the generating step (c) further comprises amplifying the adapted double-stranded Watson template with a first set of Watson-target selective primer pair, wherein the first set of Watson target-selective primer pair comprises (i) a first Watson target-selective primer comprising a sequence complementary to the 3′ adapter sequence, and (ii) a second Watson target-selective primer comprising a target-selective sequence.

In some embodiments, the second Watson target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65.

In some embodiments, the second Watson target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the generating step (d) further comprises amplifying the adapted double-stranded Crick template with a first set of Crick-target selective primer pair, wherein the first set of Crick target-selective primer pair comprises (i) a first Crick target-selective primer comprising a sequence complementary to the 3′ adapter sequence, and (ii) a second Crick target-selective primer comprising a target-selective sequence.

In some embodiments, the second Crick target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65.

In some embodiments, the second Crick target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the double-stranded DNA molecule comprises a V(D)J sequence of the immune cell receptor. In some embodiments, the target-selective sequence comprises a sequence complementary to the V(D)J sequence of the immune cell receptor. In some embodiments, the immune cell receptor comprises a B cell receptor. In some embodiments, the immune cell receptor comprises a T cell receptor.

In some embodiments, the method further comprises identifying (i) a mutation in the adapted double-stranded Watson template of the first analyte DNA family, (ii) a mutation in the adapted double-stranded Crick template of the second analyte DNA family, or (iii) a mutation in both the adapted double-stranded Watson template and the adapted double-stranded Crick template. In some embodiments, the mutation is selected from the group consisting of an insertion, a deletion, a substitution, a deletion-insertion, a duplication, an inversion, a frameshift, a repeat expansion, a translocation, and combinations thereof.

In some embodiments, the method determines the sequence of the double-stranded DNA molecule in a population of double-stranded DNA molecules by assaying both strands of the double-stranded DNA molecule. In some embodiments, a mutation in both the adapted double-stranded Watson template and the adapted double-stranded Crick template is identified.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an exemplary workflow for identification and analysis of a double-stranded DNA molecule of an immune cell.

FIG. 2 is a bar graph showing the median fraction of on-target reads (i.e., reads comprised of the intended amplicon), across the 13 J segment targets, derived from the Watson strand and Crick strand.

FIG. 3 is a bar graph showing targets exhibiting relatively uniform amplification, with coefficients of variation of 29% and 24% for the Watson and Crick-derived reads.

FIG. 4 is a bar graph showing the number of duplex UID families (i.e. each UID family represents an original molecule present in the DNA sample) being exceptionally uniform across each of the 13 targets.

FIG. 5 shows that each TRBJ primer set recovered an approximately equal number of corresponding synthetic construct molecules (median: 833.5; range: 587-1783 for average of Watson and Crick strands), wherein cross-reactive identification of non-corresponding synthetic constructs was minimal.

FIG. 6 is a graph showing the number of synthetic construct molecules identified was highly correlated to orthogonal determinations of the synthetic control construct concentrations, as measured by the number of molecules identified using the CMV-specific primer set.

FIG. 7 is a graph showing the number of synthetic construct molecules identified was highly correlated to orthogonal determinations of the synthetic control construct concentrations, as measured by the number of molecules identified by concentration in the ThermoFisher Qubit dsDNA HS assay.

FIG. 8 is a bar graph showing that the fraction of correct clonotypes identified by each primer set was high (median: 0.999; range 0.998-1.000).

FIG. 9 is a bar graph showing that the percent of sequencing reads assignable to a TCR clonotype was low for all TRBJ segments (range 0-0.06%).

FIG. 10 is a bar graph showing that the number of clonotypes identified was similarly low for all TRBJ segments (range 0-6).

FIG. 11 is a bar graph showing that the percent of sequencing reads assignable to a TCR clonotype was again low for all TRBJ segments (range 0-3.2%).

FIG. 12 is a bar graph showing that the number of clonotypes identified was also low for all TRBJ segments (range 0-82).

FIG. 13 is a bar graph showing results from evaluating the performance of the primer sets, Set 1 and Set 2, on DNA derived from T cells from a normal healthy donor, wherein the Set 1 primers had a greater percentage of sequencing reads assignable to TCR clonotypes than the Set 2 primers.

FIG. 14 is a bar graph showing results from evaluating the performance of the primer sets, Set 1 and Set 2, on DNA derived from T cells from a normal healthy donor, wherein the Set 1 primers identified a greater number of clonotypes than the Set 2 primers.

FIG. 15 is a bar graph showing results from evaluating the performance of the primer sets, Set 1 and Set 3, on DNA derived from T cells from a normal healthy donor, wherein the Set 1 primers had a greater percentage of sequencing reads assignable to TCR clonotypes than the Set 3 primers.

FIG. 16 is a bar graph showing results from evaluating the performance of the primer sets, Set 1 and Set 3, on DNA derived from T cells from a normal healthy donor, wherein the Set 1 primers identified a greater number of clonotypes than the Set 3 primers.

FIG. 17 is a bar graph showing that the Set 1 primers had a greater percentage of sequencing reads assignable to TCR clonotypes than the Set 3 primers.

FIG. 18 is a bar graph showing that the coefficient of variation for the number of on-target reads for each TRBJ segment for Multiplex Pool 1 was 103.5%.

FIG. 19 is a bar graph showing that Multiplex Pool 2 exhibited more balanced recovery of each TRBJ segment with a coefficient of variation for the number of on-target reads for TRBJ segment of 17.5%.

FIG. 20 is a bar graph showing the coefficient of variation for the number of on-target reads for each TRBJ segment for Multiplex Pool 3 was 19.4% for Watson and 21.4% for Crick, wherein Multiplex Pool 4 exhibited more balanced recovery of each TRBJ segment with a coefficient of variation for the number of on-target reads for each TRBJ segment of 13.2% for Watson and 18.1% for Crick.

FIG. 21 is a graph showing results from determining the yield with varying amounts of input DNA derived from healthy donor T cells, wherein the number of TCRs recovered, averaged from donors and replicates, was linear across input amounts from 25 ng to 400 ng.

FIG. 22 is a bar graph showing the estimated yield, averaged for donors and replicates, was also consistent across input amounts from 25 ng to 400 ng.

FIG. 23 is a graph showing results from evaluating the TCR repertoires of the cells, wherein the analysis demonstrated a reduction in clonal diversity over the course of the expansion.

FIG. 24 shows results from evaluating the TCR repertoires of the cells, the results identifying the outgrowth of specific clones over the course of expansion.

FIG. 25 shows results from evaluating the TCR repertoires of the cells, the results identifying the outgrowth of specific clones over the course of expansion.

FIG. 26 shows results from evaluating the TCR repertoires of the cells, the results identifying the outgrowth of specific clones over the course of expansion.

FIG. 27 is a graph showing results from extracting DNA from T cells derived from two healthy donors, designated “AB02” and “AB04,” and analyzing the TCR repertoires. The results show the number of TCRs recovered and the diversity of TCRs recovered was high for both donors across all replicates.

FIG. 28 is a graph showing the pairwise distance correlation between replicates from each donor was consistently high.

FIG. 29 is a graph showing the clonotype frequencies in samples from DNA input amounts of 400 ng, 100 ng, and 25 ng were well correlated for representative donor AB02.

FIG. 30 is a graph showing the clonotype frequencies in samples from DNA input amounts of 400 ng, 100 ng, and 25 ng were well correlated for representative donor AB02.

FIG. 31 is a graph showing results from analyzing the TCR V segment gene usage in the T cell populations by flow cytometry using the Beckman Coulter IOTest Beta Mark TCR VB Repertoire Kit. The proportion of V gene segment usage was well correlated with the proportion of V gene segment usage measured by flow cytometry.

FIG. 32 is a graph showing that the proportion of TCR reads corresponding to the Jurkat clone TCR was well correlated with the input DNA proportion.

FIG. 33 is a graph showing results from analyzing DNA derived from the Jurkat clonal T cell line. 1 TCR clone was identified in the Jurkat sample for 4 replicates with 2 clones called in 2 replicates.

FIG. 34 is a bar graph showing results from isolating DNA from plasma samples from patients with colorectal cancer, wherein the analysis shows the TCR repertoires in plasma.

FIG. 35 is a bar graph showing results from isolating DNA from white blood cell samples from patients with colorectal cancer, wherein the analysis shows the TCR repertoires in white blood cell samples.

FIG. 36 is a bar graph showing results from isolating DNA from tumor samples from patients with colorectal cancer, wherein the analysis shows the TCR repertoires in tumor samples.

### DETAILED DESCRIPTION

B cell (BCR) and T cell (TCR) receptors underly the function of the adaptive immune system. A large and diverse repertoire of BCR and TCR receptors is generated through somatic recombination with imprecise joining of variable (V), diversity (D), and joining (J) genes. Comprehensive characterizations of BCR and TCR repertoires is important for applications including understanding immune responses to pathogens, malignancies, and self-antigens. Tracking specific BCR and TCR sequences is also important for understanding clonal cell dynamics and responses in health and disease. Because individual clones can be rare, methods that enable accurate determination of sequences and precise quantification of sequence abundance are essential.

High throughput sequencing can be used for the characterization of TCR and BCR repertoires. Existing methods for library preparation that begin with RNA as a template generally use adapter ligation or 5′ RACE strategies. These methods can incorporate unique identifiers (UIDs) to increase accuracy. However, because cells can contain multiple BCR or TCR transcripts, quantification of clone abundance is confounded. In addition, RNA templates may not be obtainable from samples with decreased nucleotide quality, including fixed specimens. Methods that begin with DNA as a template for library preparation use multiplex PCR schemes or gene capture schemes. These methods are subject to bias from sources that include primer competition and differential amplification efficiencies. Complex methods are required to account for bias such as computational corrections, the use of spike-in standards, and primer balancing. Accordingly, existing methods for BCR and TCR sequencing are expensive, complex, require sophisticated or elaborate library preparation methods, or exhibit elements of all of these limitations. Moreover, even advanced methods still display systematic biases along with limitations in sensitivity, reproducibility, and quantification accuracy.

Accordingly, there exists a need for improvements to sequencing library preparation and workflow, to enable accurate identification of mutations, e.g., rare mutations, as well as epigenetic changes, from the same aliquot of DNA purified from clinically relevant samples.

Provided herein are methods for determining a sequence of a double stranded DNA molecule of an immune cell receptor, the method comprising: (a) attaching a 3′ adapter fragment to each 3′ end of the double-stranded DNA molecule and a 5′ adapter fragment to each 5′ end of the double-stranded DNA molecule to generate an adapted double-stranded DNA molecule, wherein the adapted double-stranded DNA molecule comprises an adapted Watson strand and an adapted Crick strand, wherein the 3′ adapter fragment comprises a molecular barcode, a primer sequence, and an adapter sequence, and wherein the molecular barcode of the adapted Watson strand is the reverse complement of the molecular barcode of the adapted Crick strand; (b) copying both strands of the adapted double-stranded DNA molecule, wherein the copying comprises performing a round of linear extension of the adapted double-stranded DNA molecule, generating an adapted double-stranded Watson template and an adapted double-stranded Crick template; (c) generating a first population of analyte DNA fragments from the adapted double-stranded Watson template and generating a first sequencing read for at least one member of the first population of analyte DNA fragments; (d) generating a second population of analyte DNA fragments from the adapted double-stranded Crick template and generating a second sequencing read for at least one member of the second population of analyte DNA fragments; (e) grouping the first sequencing reads according to the molecular barcode present on the at least one member of the first population of analyte DNA fragments to generate a first analyte DNA family; (f) grouping the second sequencing reads according to the molecular barcode present on the at least one member of the second population of analyte DNA fragments to generate a second analyte DNA family; (g) analyzing the first sequencing read of the first analyte DNA family; and (h) analyzing the second sequencing read of the second analyte DNA family, thus, determining the sequence of the double stranded DNA molecule.

Various non-limiting aspects of these methods are described herein, and can be used in any combination without limitation. Additional aspects of various components of methods for identifying the presence or absence of a mutation and methylation are known in the art.

It must be noted that, as used in the specification and the appended claims, the singular forms “a,” “an” and “the” include plural referents unless the context clearly dictates otherwise.

As used herein, an “adaptor,” an “adapter,” and a “tag” are terms that are used interchangeably, and refer to species that can be coupled to a polynucleotide sequence (e.g., in a process referred to as “tagging”) using any one of many different techniques including, but not limited to, ligation, hybridization, and tagmentation. In some embodiments, adaptors can also be nucleic acid sequences that add a function, e.g., spacer sequences, primer sequences/sites, barcode sequences, or unique molecular identifier sequences.

As used herein, the term “barcode” refers to a label, or identifier, that conveys or is capable of conveying information (e.g., information about an analyte in a sample). A barcode can be part of an analyte, or independent of an analyte. In some embodiments, a barcode can be attached to an analyte. In some embodiments, a particular barcode can be unique relative to other barcodes. In some embodiments, barcodes can have a variety of different formats. For example, barcodes can include non-random, semi-random, and/or random nucleic acid and/or amino acid sequences, and synthetic nucleic acid and/or amino acid sequences. In some embodiments, a barcode can be attached to an analyte or to another moiety or structure in a reversible or irreversible manner. In some embodiments, a barcode can be added to, for example, a fragment of a deoxyribonucleic acid (DNA) or ribonucleic acid (RNA) sample before or during sequencing of the sample. In some embodiments, barcodes can allow for identification and/or quantification of individual sequencing-reads. In some embodiments, a barcode can refer to a unique identifier (UID) and the terms “barcode” and “UID” can be used interchangeably.

As used herein, the term “nucleotides” and “nt” are used interchangeably herein to generally refer to biological molecules that comprise nucleic acids. Nucleotides can have moieties that contain the known purine and pyrimidine bases. Nucleotides may have other heterocyclic bases that have been modified. Such modifications include, e.g., methylated purines or pyrimidines, acylated purines or pyrimidines, alkylated riboses, or other heterocycles. The terms “polynucleotides,” “nucleic acid,” and “oligonucleotides” can be used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise non-naturally occurring sequences. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, a “primer” generally refers to a polynucleotide molecule comprising a nucleotide sequence (e.g., an oligonucleotide), generally with a free 3′—OH group, that hybridizes with a template sequence (such as a target polynucleotide, or a primer extension product) and is capable of promoting polymerization of a polynucleotide complementary to the template. In some embodiments, a primer is a biotinylated primer.

### Overview

This document relates to methods and materials useful for accurately identifying TCR/BCR receptor sequences present in a nucleic acid sample. In some aspects, the method comprises identifying the TCR/BCR receptor sequences by using both Watson and Crick strands of a double stranded nucleic acid template. Such methods are particularly useful for characterizing and quantifying TCR/BCR receptor sequences, and allowing for the identification of TCR and BCR repertoires with high confidence.

In some cases, the methods and materials described herein can determine TCR/BCR receptor sequences with a low error rate. For example, the methods and materials described herein can be used to determine TCR/BCR receptor sequences in a nucleic acid template with an error rate of less than about 1% (e.g., less than about 0.1%, less than about 0.05%, or less than about 0.01%). In some cases, the methods and materials described herein can be used to determine TCR/BCR receptor sequences in a nucleic acid template with an error rate of from about 0.001% to about 0.01%. In some cases, the error rate associated with the identification of TCR/BCR receptor sequences in analyte DNA fragments according to a method described herein is no more than 1×10⁻², no more than 1×10⁻³, no more than 1×10⁻⁴, no more than 1×10⁻⁵, no more than 1×10⁻⁶, no more than 5×10⁻⁶, or no more than 1×10⁻⁷. In some cases, the error rate associated with the identification of TCR/BCR receptor sequences in analyte DNA fragments according to a method described herein is reduced by at least 2-fold, 4-fold, 5-fold, 10-fold, 20-fold, 30-fold, 40-fold, 50-fold, 60-fold, 70-fold, 80-fold, 90-fold, or 100-fold, as compared to an alternative method of identifying TCR/BCR receptor sequences that does not require the use of both Watson and Crick strands of an analyte DNA fragment.

In some embodiments, the alternative method comprises standard molecular barcoding or standard PCR-based molecular barcoding followed by sequencing. In particular embodiments, the alternative method comprises: (a) attaching adapters to a population of double-stranded DNA fragments in an analyte DNA sample, wherein the adapters comprise a unique exogenous UID; (b) performing an initial amplification to amplify the adapter-ligated, double-stranded DNA fragments to produce amplicons; (c) determining sequence reads of one or more amplicons of the one or more of the adapter-ligated, double-stranded DNA fragments; (d) assigning the sequence reads into UID families, wherein each member of a UID family comprises the same exogenous UID sequence; (e) identifying a nucleotide sequence as accurately representing an analyte DNA fragment when a threshold percentage of members of a UID family contain the sequence; and (f) identifying TCR/BCR receptor sequences in the analyte DNA fragment.

In some cases, the methods and materials described herein can be used to achieve efficient duplex recovery. For example, methods described herein can be used to recover PCR amplification products derived from both the Watson strand and the Crick strand of a double stranded nucleic acid template. In some cases, the methods described herein can be used to achieve at least 50% (e.g., about 50%, about 60%, about 70%, about 75%, about 80%, about 82%, about 85%, about 88%, about 90%, about 93%, about 95%, about 97%, about 99%, or 100%) duplex recovery.

In some cases, the methods and materials described herein can be used to determine TCR/BCR receptor sequences having low allele frequency. For example, methods described herein can be used to determine TCR/BCR receptor sequences having low allele frequency of less than about 1% (e.g., less than about 0.1%, less than about 0.05%, or less than about 0.01%). In some cases, the methods described herein can be used to determine TCR/BCR receptor sequences having low allele frequency of about 0.001%.

In some cases, the methods described herein can be used to determine TCR/BCR receptor sequences that are present in an analyte nucleic acid sample at a frequency of 0.1% or less. In some embodiments, the methods described herein can be used to determine TCR/BCR receptor sequences that are present in an analyte nucleic acid sample at a frequency of 0.1% to 0.00001%. In some embodiments, the methods described herein can be used to determine TCR/BCR receptor sequences that are present in an analyte nucleic acid sample at a frequency of 0.1% to 0.01%.

In some cases, methods for determining TCR/BCR receptor sequences of a double stranded nucleic acid can include generating a duplex sequencing library having a duplex molecular barcode on each end (e.g., the 5′ end and the 3′ end) of each nucleic acid in the library, generating a library of single stranded Watson strand-derived sequences and a library of single stranded Crick-strand derived sequences from the duplex sequencing library, and determining TCR/BCR receptor sequences of the double stranded nucleic acid in each single stranded library. The presence of a first molecular barcode in a 3′ duplex adapter and a second molecular barcode present in a 5′ adapter can be used to distinguish amplification products derived from the Watson strand from amplification products derived from the Crick strand.

In some cases, methods for identifying TCR/BCR receptor sequences comprise: (a) attaching partially double-stranded 3′ adapters to 3′ ends of both Watson and Crick strands of a population of double-stranded DNA fragments in an analyte DNA sample, wherein a first strand of the partially double-stranded 3′ adapter comprises, in the 5′-3′ direction, (i) a first segment, (ii) an exogenous UID sequence, (iii) an annealing site for a 5′ adapter, and (iv) a universal 3′ adapter sequence comprising an R2 sequencing primer site, and wherein the second strand of the partially double-stranded 3′ adapter comprises, in the 5′ to 3′ direction, (i) a segment complementary to the first segment, and (ii) a 3′ blocking group, optionally wherein the second strand is degradable; (b) annealing 5′ adapters to the 3′ adapters via the annealing site, wherein the 5′ adapters comprise, in the 5′ to 3′ direction, (i) a universal 5′ adapter sequence that is not complementary to the universal 3′ adapter sequence and that comprises an R1 sequencing primer site, and (ii) a sequence complementary to the annealing site for the 5′ adapter; (c) performing a nick translation reaction to extend the 5′ adapters across the exogenous UID sequence of the 3′ adapters and covalently link the extended 5′ adapter to the 5′ ends of the Watson and Crick strands of the double-stranded DNA fragments; (d) performing an initial amplification to amplify the adapter-ligated, double-stranded DNA fragments to produce amplicons; (e) determining sequence reads of one or more amplicons of the one or more of the adapter-ligated, double-stranded DNA fragments; (f) assigning the sequence reads into UID families, wherein each member of a UID family comprises the same exogenous UID sequence; (g) assigning sequence reads of each UID family into a Watson subfamily and Crick subfamily based on spatial relationship of the exogenous UID sequence to the R1 and R2 read sequence; (h) identifying a nucleotide sequence as accurately representing a Watson strand of an analyte DNA fragment when a threshold percentage of members of the Watson subfamily contain the sequence; (i) identifying a nucleotide sequence as accurately representing a Crick strand of an analyte DNA fragment when a threshold percentage of members of the Crick subfamily contain the sequence; (j) identifying TCR/BCR receptor sequences in the nucleotide sequence accurately representing the Watson Strand; (k) identifying TCR/BCR receptor sequences in the nucleotide sequence accurately representing the Crick Strand; and (1) identifying TCR/BCR receptor sequences in the analyte DNA fragment when the TCR/BCR receptor sequences in the nucleotide sequence accurately representing the Watson strand and the TCR/BCR receptor sequences in the nucleotide sequence accurately representing the Crick strand are the same TCR/BCR receptor sequences.

In some cases, methods for identifying TCR/BCR receptor sequences comprises: (a) attaching adapters to a population of double-stranded DNA fragments, wherein the adapters comprise a double-stranded portion comprising an exogenous UID and a forked portion comprising (i) a single-stranded 3′ adapter sequence comprising an R2 sequencing primer site and (ii) a single-stranded 5′ adapter sequence comprising an R1 sequencing primer site; (b) performing an initial amplification to amplify the adapter-ligated, double-stranded DNA fragments to produce amplicons; (c) selectively amplifying amplicons of Watson strands comprising the target polynucleotide sequence with a first set of Watson target-selective primer pairs, the first set of Watson target-selective primer pairs comprising: (i) a first Watson target-selective primer comprising a sequence complementary to the R2 sequencing primer site of the universal 3′ adapter sequence, and (ii) a second Watson target-selective primer comprising a target-selective sequence, thereby creating target Watson amplification products; (d) selectively amplifying amplicons of Crick strands comprising the same target polynucleotide sequence with a first set of Crick target-selective primer pairs, the first set of Crick target-selective primer pairs comprising: a first Crick target-selective primer comprising a sequence complementary to the R1 sequencing primer site of the universal 5′ adapter sequence, and (ii) a second Crick target-selective primer comprising the same target-selective sequence as the second Crick target-selective primer sequence, thereby creating target Crick amplification products; (e) determining sequence reads of the target Watson amplification products and the target Crick amplification products; (f) assigning the sequence reads into UID families, wherein each member of a UID family comprises the same exogenous UID sequence; (g) assigning sequence reads of each UID family into a Watson subfamily and Crick subfamily based on spatial relationship of the exogenous UID sequence to the R1 and R2 read sequence; (h) identifying a nucleotide sequence as accurately representing a Watson strand of an analyte DNA fragment when a threshold percentage of members of the Watson family contain the sequence; (i) identifying a nucleotide sequence as accurately representing a Crick strand of an analyte DNA fragment when a threshold percentage of members of the Crick family contain the sequence; and (j) identifying TCR/BCR receptor sequences in the analyte DNA fragment when the nucleotide sequence accurately representing the Watson strand and the nucleotide sequence accurately representing the Crick strand both contain the same TCR/BCR receptor sequences.

In some cases, the methods and materials described herein can be used to independently assess each strand of a double stranded nucleic acid. For example, when a nucleic acid mutation is identified in independently assessed strands of a double stranded nucleic acid as described herein, the materials and methods described herein can used to determine from which strand of the double stranded nucleic acid the nucleic acid mutation originated.

Any appropriate method can be used to generate a duplex sequencing library. As used herein a duplex sequencing library is a plurality of nucleic acid fragments including a duplex molecular barcode on at one end (e.g., the 5′ end and/or the 3′ end) of each nucleic acid fragment in the library and can allow both strands of a double stranded nucleic acid to be sequenced. In some cases, a nucleic acid sample can be fragmented to generate nucleic acid fragments, and the generated nucleic acid fragments can be used to generate a duplex sequencing library. Nucleic acid fragments used to generate a duplex sequencing library can also be referred to herein as input nucleic acid. For example, when nucleic acid fragments used to generate a duplex sequencing library are DNA fragments, the DNA fragments can also be referred to herein as input DNA. A duplex sequencing library can include any appropriate number of nucleic acid fragments. In some cases, generating a duplex sequencing library can include fragmenting a nucleic acid template and ligating adapters to each end of each nucleic acid fragment in the library.

### Analyte Nucleic Acids

Nucleic acid templates in an analyte nucleic acid sample can comprise any type of nucleic acid (e.g., DNA, RNA, and DNA/RNA hybrids). In some cases, a nucleic acid template can be a double-stranded DNA template. Examples of nucleic acid can be used as a template for the methods described herein include, without limitation, genomic DNA, circulating free DNA (cfDNA; e.g., circulating tumor DNA (ctDNA), and cell-free fetal DNA (cffDNA)).

In some embodiments, the nucleic acid templates in the nucleic acid sample are nucleic acid fragments, e.g., DNA fragments. In some embodiments, the ends of a DNA fragment represent unique sequences which can be used as an endogenous unique identifier of the fragment. In some embodiments, the fragments are manually produced. In some embodiments, the fragments are produced by shearing, e.g., enzymatic shearing, shearing by chemical means, acoustic shearing, nebulization, centrifugal shearing, point-sink shearing, needle shearing, sonication, restriction endonucleases, non-specific nucleases (e.g., DNase I), and the like. In some embodiments, the fragments are not manually produced. In some embodiments, the fragments are from a cfDNA sample.

In some embodiments, a nucleic acid fragment to be analyzed has a length of about 4 to about 1000 nucleotides (e.g., about 10 to about 1000, about 20 to about 1000, about 30 to about 1000, about 40 to about 1000, about 50 to about 1000, about 60 to about 1000, about 70 to about 1000, about 80 to about 1000, about 90 to about 1000, about 100 to about 1000, about 250 to about 1000, about 500 to about 1000, about 750 to about 1000, about 4 to about 750, about 10 to about 750, about 20 to about 750, about 30 to about 750, about 40 to about 750, about 50 to about 750, about 60 to about 750, about 70 to about 750, about 80 to about 750, about 90 to about 750, about 100 to about 750, about 250 to about 750, about 500 to about 750, about 4 to about 500, about 10 to about 500, about 20 to about 500, about 30 to about 500, about 40 to about 500, about 50 to about 500, about 60 to about 500, about 70 to about 500, about 80 to about 500, about 90 to about 500, about 100 to about 500, about 250 to about 500, about 4 to about 250, about 10 to about 250, about 20 to about 250, about 30 to about 250, about 40 to about 250, about 50 to about 250, about 60 to about 250, about 70 to about 250, about 80 to about 250, about 90 to about 250, about 100 to about 250, about 4 to about 100, about 10 to about 100, about 20 to about 100, about 30 to about 100, about 40 to about 100, about 50 to about 100, about 60 to about 100, about 70 to about 100, about 80 to about 100, about 90 to about 100, about 4 to about 90, about 10 to about 90, about 20 to about 90, about 30 to about 90, about 40 to about 90, about 50 to about 90, about 60 to about 90, about 70 to about 90, about 80 to about 90, about 4 to about 80, about 10 to about 80, about 20 to about 80, about 30 to about 80, about 40 to about 80, about 50 to about 80, about 60 to about 80, about 70 to about 80, about 4 to about 70, about 10 to about 70, about 20 to about 70, about 30 to about 70, about 40 to about 70, about 50 to about 70, about 60 to about 70, about 4 to about 60, about 10 to about 60, about 20 to about 60, about 30 to about 60, about 40 to about 60, about 50 to about 60, about 4 to about 50, about 10 to about 50, about 20 to about 50, about 30 to about 50, about 40 to about 50, about 4 to about 40, about 10 to about 40, about 20 to about 40, about 30 to about 40, about 4 to about 30, about 10 to about 30, about 20 to about 30, about 4 to about 20, about 10 to about 20, or about 4 to about 10). In some embodiments, the length of the nucleic acid fragment to be analyzed may be less than 1000 (e.g., less than 750, less than 500, less than 250, less than 100, less than 50, or less than 20) nucleotides.

In some embodiments, ends of nucleic acid templates are used as endogenous UIDs. A skilled artisan may determine the length of the endogenous UID needed to uniquely identify a nucleic acid template, using factors such as, e.g., overall template length, complexity of nucleic acid templates in a partition or starting nucleic acid sample, and the like. In some embodiments, 10-500 nucleotides of the ends of nucleic acid templates are used as endogenous UIDs. In some embodiments, 15-100 nucleotides of the ends of nucleic acid templates are used as endogenous UIDs. In some embodiments, 15-40 nucleotides of the ends of nucleic acid templates are used as endogenous UIDs. In some embodiments, at least 10 nucleotides of the ends of nucleic acid templates are used as endogenous UIDs. In some embodiments, at least 15 nucleotides of the ends of nucleic acid templates are used as endogenous UIDs. In some embodiments, only one end of a nucleic acid template is used as an endogenous UID.

In some embodiments, nucleic acid templates comprise one or more target polynucleotides. The terms “target polynucleotide,” “target region,” “nucleic acid template of interest,” “desired locus,” “desired template,” or “target,” are used interchangeably herein to refer to a polynucleotide of interest under study. In certain embodiments, a target polynucleotide contains one or more sequences that are of interest and under study. A target polynucleotide can comprise, for example, a genomic sequence. The target polynucleotide can comprise a target sequence whose presence, amount, and/or nucleotide sequence, or changes in these, are desired to be determined.

The target polynucleotide can be a region of gene associated with a disease. In some embodiments, the gene is a druggable target. The term “druggable target”, as used herein, generally refers to a gene or cellular pathway that is modulated by a disease therapy. The disease can be cancer. Accordingly, the gene can be a known cancer-related gene.

In some embodiments, the input nucleic acid, also referred to herein as the nucleic acid sample, was obtained from a biological sample. The biological sample may be obtained from a subject. In some embodiments, the subject is a mammal. Examples of mammals from which nucleic acid can be obtained and used as a nucleic acid template in the methods described herein include, without limitation, humans, non-human primates (e.g., monkeys), dogs, cats, sheep, rabbits, mice, hamsters, and rats. In some embodiments, the subject is a human subject. In some embodiments, the subject is a plant.

Biological samples include but are not limited to plasma, serum, blood, tissue, tumor sample, stool, sputum, saliva, urine, sweat, tears, ascites, bronchoaveolar lavage, semen, archeologic specimens and forensic samples. In particular embodiments, the biological sample is a solid biological sample, e.g., a tumor sample. In some embodiments, the solid biological sample is processed. The solid biological sample may be processed by fixation in a formalin solution, followed by embedding in paraffin (e.g., is a FFPE sample). Processing can alternatively comprise freezing of the sample prior to conducting the probe-based assay. In some embodiments, the sample is neither fixed nor frozen. The unfixed, unfrozen sample can be, by way of example only, stored in a storage solution configured for the preservation of nucleic acid.

In some embodiments, the biological sample is a liquid biological sample. Liquid biological samples include, but are not limited to plasma, serum, blood, sputum, saliva, urine, sweat, tears, ascites, bronchoaveolar lavage, and semen. In some embodiments, the liquid biological sample is cell free or substantially cell free. In particular embodiments, the biological sample is a plasma or serum sample. In some embodiments, the liquid biological sample is a whole blood sample. In some embodiments, the liquid biological sample comprises peripheral mononuclear blood cells.

In some embodiments, a nucleic acid sample has been isolated and purified from the biological sample. Nucleic acid can be isolated and purified from the biological sample using any means known in the art. For example, a biological sample may be processed to release nucleic acid from cells, or to separate nucleic acids from unwanted components of the biological sample (e.g., proteins, cell walls, other contaminants). For example, nucleic acid can be extracted from the biological sample using liquid extraction (e.g., Trizol, DNAzol) techniques. Nucleic acid can also be extracted using commercially available kits (e.g., Qiagen DNeasy kit, QIAamp kit, Qiagen Midi kit, QIAprep spin kit).

In some embodiments, the biological sample comprises low amounts of nucleic acid. In some embodiments, the biological sample comprises less than about 500 nanograms (ng) of nucleic acid. For example, the biological sample comprises from about 30 ng to about 40 ng of nucleic acid.

Nucleic acid can be concentrated by known methods, including, by way of example only, centrifugation. Nucleic acid can be bound to a selective membrane (e.g., silica) for the purposes of purification. Nucleic acid can also be enriched for fragments of a desired length, e.g., fragments which are less than 1000, 500, 400, 300, 200 or 100 base pairs in length. Such an enrichment based on size can be performed using, e.g., PEG-induced precipitation, an electrophoretic gel or chromatography material (Huber et al. (1993) Nucleic Acids Res. 21:1061-6), gel filtration chromatography, TSK gel (Kato et al. (1984) J. Biochem, 95:83-86), which publications are hereby incorporated by reference.

Polynucleotides extracted from a biological sample can be selectively precipitated or concentrated using any methods known in the art.

In some embodiments, the nucleic acid sample comprises less than about 35 ng of nucleic acid. For example, the nucleic acid sample comprises can include from about 1 ng to about 35 ng of nucleic acid (e.g., from about 1 ng to about 30 ng, from about 1 ng to about 25 ng, from about 1 ng to about 20 ng, from about 1 ng to about 15 ng, from about 1 ng to about 10 ng, from about 1 ng to about 5 ng, from about 5 ng to about 35 ng, from about 10 ng to about 35 ng, from about 15 ng to about 35 ng, from about 20 ng to about 35 ng, from about 25 ng to about 35 ng, from about 30 ng to about 35 ng, from about 5 ng to about 30 ng, from about 10 ng to about 25 ng, from about 15 ng to about 20 ng, from about 5 ng to about 10 ng, from about 10 ng to about 15 ng, from about 15 ng to about 20 ng, from about 20 ng to about 25 ng, or from about 25 ng to about 30 ng of nucleic acid). In some cases, a nucleic acid sample can include nucleic acid from a genome that includes more than about several hundred nucleotides of nucleic acid.

In some cases, a nucleic acid sample can be essentially free of contamination. For example, when a nucleic acid sample is a cfDNA template, the cfDNA can be essentially free of genomic DNA contamination. In some cases, a cfDNA sample that is essentially free of genomic DNA contamination can include minimal (or no) high molecular weight (e.g., >1000 bp) DNA. In some cases, methods described herein can include determining whether a nucleic acid sample is essentially free of contamination. Any appropriate method can be used to determine whether a nucleic acid sample is essentially free of contamination. Examples of methods that can be used to determine whether a nucleic acid sample is essentially free of contamination include, for example, a TapeStation system, and a Bioanalyzer. For example, when using a TapeStation system and/or a Bioanalyzer to determine whether a cfDNA sample is essentially free of genomic DNA contamination, a prominent peak at ˜180 bp (e.g., corresponding to mononucleosomal DNA) can be used to indicate that the nucleic acid sample is essentially free of genomic DNA contamination.

In some cases, nucleic acid fragments that can be used to generate a duplex sequencing library (e.g., prior to attaching a 3′ duplex adapter to the 3′ ends of the nucleic acid fragments) can be end-repaired. Any appropriate method can be used to end-repair a nucleic acid template. For example, blunting reactions (e.g., blunt end ligations) and/or dephosphorylation reactions can be used to end-repair a nucleic acid template. In some cases, blunting can include filling in a single stranded region. In some cases, blunting can include degrading a single stranded region. In some cases, blunting and dephosphorylation reactions can be used to end-repair a nucleic acid template.

### Adapters

As used herein, an “adapter” and “adapter fragment” can refer to a species that can be coupled to a polynucleotide sequence using any one of many different techniques including, but not limited to, ligation, hybridization, and tagmentation. In some embodiments, adapter fragments can also be nucleic acid sequences that add a function, e.g., spacer sequences, primer sequences/sites, or barcode sequences (e.g., UID sequences).

In some embodiments, the method comprises attaching adapters to a population of double-stranded DNA molecules to produce a population of adapter-attached, double-stranded DNA molecules, wherein the adapted double-stranded DNA molecule comprises an adapted Watson strand and an adapted Crick strand, wherein the adapter fragment comprises a molecular barcode, a primer sequence, and an adapter sequence, and wherein the molecular barcode of the adapted Watson strand is the reverse complement of the molecular barcode of the adapted Crick strand. In some embodiments, the primer sequence can be the reverse complement of the adapter sequence. In some embodiments, the adapter sequence can include specific sequences to allow sequencing when generating a sequence library. In some embodiments, the adapter sequence comprises a sequencing primer sequence (e.g., R1, R2).

In some embodiments, the adapters comprise a double-stranded portion comprising an exogenous UID and a forked portion comprising (i) a single-stranded 3′ adapter sequence and (ii) a single-stranded 5′ adapter sequence. In some embodiments, the single-stranded 3′ adapter sequence is not complementary to the single-stranded 5′ adapter sequence. In some embodiments, the 3′ adapter sequence comprises a second (e.g., R2) sequencing primer site and the 5′ adapter sequence comprises a first (e.g., R1) sequencing primer site. It is to be understood that an “R1” and “R2” sequencing primer sites are used by sequencing systems that produce paired end reads, e.g., reads from opposite ends of a DNA fragment to be sequenced. In some embodiments, the R1 sequencing primer is used to produce a first population of reads from first ends of DNA fragments, and the R2 sequencing primer is used to produce a second population of reads from the opposite ends of the DNA fragments. The first population is referred to herein as “R1” or “Read 1” reads. The second population is referred to herein as “R2” or “Read 2” reads. The R1 and R2 reads can be aligned as “read pairs” or “mate pairs” corresponding to each strand of a double-stranded analyte DNA fragment.

Certain sequencing systems, e.g., Illumina, utilizes what they refer to as “R1” and “R2” primers, and “R1” and “R2” reads. It should be noted that the terms “R1” and “R2”, and “Read 1” and “Read 2”, for the purposes of this application, are not limited to how they are referenced in relation to a particular sequencing platform. For example, if an Illumina sequencer is used, the “R2” primer and corresponding R2 read disclosed herein may refer to the Illumina “R2” primer and read, or may refer to the Illumina “R1” primer and read, so long as the “R1” primer and corresponding R1 read disclosed herein refers to the other Illumina primer and read. To clarify, in some embodiments wherein an “R2” primer provided herein is the Illumina “R1” primer producing “R1” reads, the corresponding “R1” primer provided herein is the Illumina “R2” primer producing “R2” reads. To clarify, in some embodiments wherein an “R2” primer provided herein is the Illumina “R2” primer providing “R2” reads, the “R1” primer provided herein is the Illumina “R1” primer providing R1 reads.

In some embodiments, the exogenous UID is unique to each double-stranded DNA fragment in the nucleic acid sample. In some embodiments, the exogenous UID is not unique to each double-stranded DNA fragment.

In some embodiments, the exogenous UID has a length. The length can be about 2-4000 nt. The length can be about 6-100 nt. The length can be about 8-50 nt. The length can be about 10-20 nt. The length can be about 12-14 nt. In some embodiments, the length of the exogenous UID is sufficient to uniquely barcode the molecules and the length/sequence of the exogenous UID does not interfere with the downstream amplification steps.

In some embodiments, the exogenous UID sequence does not exist in the nucleic acid template. In some embodiments, the exogenous UID sequence does not exist in a desired template harboring a desired locus. Such unique sequences can be randomly generated, e.g., by a computer readable medium, and selected by BLASTing against known nucleotide databases such as, e.g., EMBL, GenBank, or DDBJ. In some embodiments, an exogenous UID sequence exists in a nucleic acid template. In such cases, the position of the exogenous UID sequence in the sequence read is used to distinguish the exogenous UID sequence from a sequence within the nucleic acid template.

In some embodiments, the exogenous UID sequence is random. In some embodiments, the exogenous UID sequence is a random N-mer. For example, if the exogenous UID sequence has a length of six nt, then it may be a random hexamer. If the exogenous UID sequence has a length of 12 nt, then it may be a random 12-mer.

Exogenous UIDs may be made using random addition of nucleotides to form a sequence having a length to be used as an identifier. At each position of addition, a selection from one of four deoxyribonucleotides may be used. Alternatively a selection from one of three, two, or one deoxyribonucleotides may be used. Thus the UID may be fully random, somewhat random, or non-random in certain positions.

In some embodiments, the exogenous UIDs are not random N-mers, but are selected from a predetermined set of exogenous UID sequences.

Exemplary exogenous UIDs suitable for use in the methods disclosed herein are described in PCT/US2012/033207, which is hereby incorporated by reference in its entirety.

Forked adapters described herein may be attached to double-stranded DNA fragments by any means known in the art.

In some embodiments, the forked adapters are attached to double-stranded DNA fragments by: (a) attaching partially double-stranded 3′ adapters to 3′ ends of both Watson and Crick strands of a population of double-stranded DNA fragments, wherein a first strand of the partially double-stranded 3′ adapter comprises, in the 5′-3′ direction, (i) a first segment, (ii) an exogenous UID sequence, (iii) an annealing site for a 5′ adapter, and (iv) a universal 3′ adapter sequence comprising an R2 sequencing primer site, and wherein the second strand of the partially double-stranded 3′ adapter comprises, in the 5′ to 3′ direction, (i) a segment complementary to the first segment, and (ii) a 3′ blocking group, optionally wherein the second strand is degradable; (b) annealing 5′ adapters to the 3′ adapters via the annealing site, wherein the 5′ adapters comprise, in the 5′ to 3′ direction, (i) a universal 5′ adapter sequence that is not complementary to the universal 3′ adapter sequence and that comprises an R1 sequencing primer site, and (ii) a sequence complementary to the annealing site for the 5′ adapter; and (c) performing a nick translation reaction to extend the 5′ adapters across the exogenous UID sequence of the 3′ adapters and covalently link the extended 5′ adapter to the 5′ ends of the Watson and Crick strands of the double-stranded DNA fragments.

In some embodiments, the forked adapters are attached to double-stranded DNA fragments by: (a) attaching a 3′ duplex adapter to 3′ ends of both Watson and Crick strands of a population of double-stranded DNA fragments. A 3′ duplex adapter, also referred to herein as a partially double stranded 3′ adapter, as described herein is an oligonucleotide complex including a molecular barcode that can have a first oligonucleotide (also referred to herein as “first strand”) annealed (hybridized) to a second oligonucleotide (also referred to herein as “second strand”) such that a portion (e.g., first portion) of the 3′ duplex adapter is double stranded and a portion (e.g., a second portion) of the 3′ duplex adapter is single stranded. In some cases, a first oligonucleotide of a 3′ duplex adapter described herein comprises a first segment comprising nucleotides that are complementary to nucleotides present in a second oligonucleotide of the 3′ duplex adapter (e.g., such that the first oligonucleotide of the 3′ duplex adapter and the second oligonucleotide of the 3′ duplex adapter can anneal at the complementary region).

The first oligonucleotide of a 3′ duplex adapter described herein can be an oligonucleotide that includes a 5′ phosphate and a molecular barcode. The first oligonucleotide of a 3′ duplex adapter described herein can include any appropriate number of nucleotides. Any appropriate molecular barcode can be included in a first oligonucleotide of a 3′ duplex adapter described herein. In some cases, a molecular barcode can include a random sequence. In some cases, a molecular barcode can include a fixed sequence. Examples of molecular barcodes that can be included in a first oligonucleotide of a 3′ duplex adapter described herein include, without limitation, IDT 8, IDT 10, ILMN 8, ILMN 10 as available from Integrated DNA technologies. Any appropriate type of molecular barcode can be used. In some cases, a molecular barcode comprise an exogenous UID sequence. Exogenous UIDs are described herein. Examples of oligonucleotides that include a 5′ phosphate and a molecular barcode and can be included in a first oligonucleotide of a 3′ duplex adapter described herein include, without limitation, ATAAAACGACGGCNNNNNNNNNNNNNNAGATCGGAAGAGCACACGTCTGAACTCCA G*T*C (with the asterisks representing phosphorothioate bonds; SEQ ID NO: 1), where NNNNNNNNNNNNNN (SEQ ID NO: 2) is a molecular barcode, and where the number of nucleotides in the molecular barcode can be from 0 to about 25.

In some embodiments, the first oligonucleotide of the 3′ duplex adapter comprises an annealing site for a 5′ adapter.

In some embodiments, the first oligonucleotide of the 3′ duplex adapter comprises a universal 3′ adapter sequence. In some embodiments, the universal 3′ adapter sequence comprises an R2 sequencing primer site.

In some cases, a first oligonucleotide of a 3′ duplex adapter described herein also can include one or more features to prevent or reduce extension during a PCR. A feature that can prevent or reduce extension during a PCR can be any type of feature (e.g., a chemical modification). Examples of feature that can prevent or reduce extension during a PCR and can be included in a first oligonucleotide of a 3′ duplex adapter described herein include, without limitation, 3SpC3 and 3Phos. A feature that can prevent or reduce extension during a PCR can be incorporated into a first oligonucleotide of a 3′ duplex adapter described herein in any appropriate position within the oligonucleotide. In some case, a molecule that can prevent or reduce extension during a PCR can be incorporated internally within the oligonucleotide. In some case, a molecule to prevent or reduce extension during a PCR can be incorporated at and end (e.g., the 5′ end) of the oligonucleotide.

In particular embodiments, the first oligonucleotide of the 3′ duplex adapter comprises a 5′ phosphate, a first segment comprising nucleotides that are complementary to nucleotides present in a second oligonucleotide of the 3′ duplex adapter, an exogenous UID sequence, an annealing site for a 5′ adapter, and a universal 3′ adapter sequence.

The second oligonucleotide of a 3′ duplex adapter described herein can be an oligonucleotide that includes a blocked 3′ group (e.g., to reduce or eliminate dimerization of two adapters). The second oligonucleotide of a 3′ duplex adapter described herein can include any appropriate number of nucleotides. In some embodiments, the second oligonucleotide of the 3′ duplex adapter is complementary to the first segment of the first oligonucleotide of the 3′ duplex adapter. An exemplary oligonucleotide that includes a blocked 3′ group and can be included in a second oligonucleotide of a 3′ duplex adapter described herein includes, without limitation, GCCGUCGUUUUAdT (SEQ ID NO: 3).

The second oligonucleotide of a 3′ duplex adapter described herein can be degradable. Any appropriate method can be used to degrade a second oligonucleotide of a 3′ duplex adapter described herein. For example, UDG can be used to degrade a second oligonucleotide of a 3′ duplex adapter described herein.

In some cases, a 3′ duplex adapter described herein can include a first oligonucleotide including the sequence ATAAAACGACGGCNNNNNNNNNNNNNNAGATCGGAAGAGCACACGTCTGAACTCCA 5G*T*C/3SpC3 (SEQ ID NO: 1) annealed to a second oligonucleotide including the sequence GCCGUCGUUUUAdT (SEQ ID NO: 3).

In some cases, a 3′ duplex adapter described herein can include a commercially available adapter. An exemplary commercially available adapters that can be used as (or can be used to generate) a 3′ duplex adapter described herein includes, without limitation, adapters in an Accel-NGS 2S DNA Library Kit (Swift Biosciences, cat. #21024).

The 3′ adapters can be attached (e.g., covalently attached) to 3′ ends of the double-stranded DNA fragments using any appropriate method. In some embodiments, the 3′ adapters are attached by ligation. In some embodiments, the ligation comprises use of a ligase. Examples of ligases that can be used to attach a 3′ adapter to the 3′ ends of each nucleic acid fragment include, without limitation, T4 DNA ligases, E. coli ligases (e.g., Enzyme Y3), CircLigase I, CircLigase II, Taq-Ligase, T3 Ligase, T7 Ligase, and 9N Ligase.

Once the 3′ duplex adapter is attached (e.g., covalently attached) to the 3′ ends of each nucleic acid fragment, the second oligonucleotide of a 3′ duplex adapter described herein can be degraded, and a 5′ adapter can be attached (e.g., covalently attached) to the 5′ ends of each nucleic acid fragment. In some embodiments, the 5′ adapter sequence is not complementary to the first oligonucleotide of the 3′ adapter. In some embodiments, the 5′ adapter sequence comprises, in the 5′ to 3′ direction, an R1 sequencing primer site and a sequence complementary to the annealing site of the 3′ adapter.

In some embodiments, the attaching of the 5′ adapter comprises annealing the 5′ adapter to the 3′ adapter via the annealing site.

A 5′ adapter can anneal to a nucleic acid fragment upstream of a molecular barcode on a 3′ duplex adapter such that a gap (e.g., single stranded nucleic acid fragment) containing a portion (e.g., a molecular barcode) of the 3′ duplex adapter is present on the nucleic acid fragment. The gap containing a portion of the 3′ duplex adapter can be filled in (e.g., to generate a double stranded nucleic acid fragment). Any appropriate method can be used to fill in the single stranded gap. Examples of methods that can be used to fill in a single stranded gap on a nucleic acid fragment include, without limitation, polymerases such as DNA polymerases (e.g., Taq polymerases such as a Taq-B polymerase) and nick-translation reactions (e.g., including both a ligase such as an E. coli ligase and a polymerase such as a DNA polymerase). In cases where filling in a single stranded gap on a nucleic acid fragment includes providing a polymerase, the method also can include providing deoxyribonucleotide triphosphates (dNTPs; e.g., dATP, dGTP, dCTP, and dTTP). In some cases, attaching a 5′ adapter to the 5′ ends of each nucleic acid fragment and filling in the single stranded gap can be done concurrently (e.g., in a single reaction tube).

In some cases, alternative methods can be used to attach the adapters to templates. For example, nucleic acid fragments can be treated with single strand nucleases (e.g., to digest overhangs) followed by ligation can be used to prepare a duplex sequencing library. For example, a single nucleotide can be added to the 3′ ends of each nucleic acid fragment and adapters (e.g., containing a molecular barcode) containing a complementary base at the 5′ end can be ligated to each nucleic acid fragment to prepare a duplex sequencing library of adapter-attached templates.

### Molecular Barcode

As used herein, “molecular barcode” refers to a barcode that serves to identify individual nucleic acid fragments in an original sample prior to barcoding and amplification. In some embodiments, each individual nucleic acid fragment will have a unique molecular barcode. In some embodiments, barcodes may be randomly generated nucleotide sequences or intentionally chosen nucleotide runs. For attaching molecular barcodes in particular, the number of individual molecular barcodes in a reaction mixture will be in excess of the number of nucleic acid fragments.

In some embodiments, a molecular barcode is unique to each double-stranded DNA fragment in the nucleic acid sample. In some embodiments, the molecular barcode includes an endogenous barcode, an exogenous barcode, or both.

In some embodiments, the molecular barcode has a length of about 2 to about 4000 (e.g., about 2 to about 3500, about 2 to about 3000, about 2 to about 2500, about 2 to about 2000, about 2 to about 1500, about 2 to about 1000, about 2 to about 500, about 2 to about 100, about 2 to about 50, about 2 to about 20, about 2 to about 10, about 10 to about 4000, about 10 to about 3500, about 10 to about 3000, about 10 to about 2500, about 10 to about 2000, about 10 to about 1500, about 10 to about 1000, about 10 to about 500, about 10 to about 100, about 10 to about 50, about 10 to about 20, about 20 to about 4000, about 20 to about 3500, about 20 to about 3000, about 20 to about 2500, about 20 to about 2000, about 20 to about 1500, about 20 to about 1000, about 20 to about 500, about 20 to about 100, about 20 to about 50, about 50 to about 4000, about 50 to about 3500, about 50 to about 3000, about 50 to about 2500, about 50 to about 2000, about 50 to about 1500, about 50 to about 1000, about 50 to about 500, about 50 to about 100, about 100 to about 4000, about 100 to about 3500, about 100 to about 3000, about 100 to about 2500, about 100 to about 2000, about 100 to about 1500, about 100 to about 1000, about 100 to about 500, about 500 to about 4000, about 500 to about 3500, about 500 to about 3000, about 500 to about 2500, about 500 to about 2000, about 500 to about 1500, about 500 to about 1000, about 1000 to about 4000, about 1000 to about 3500, about 1000 to about 3000, about 1000 to about 2500, about 1000 to about 2000, about 1000 to about 1500, about 1500 to about 4000, about 1500 to about 3500, about 1500 to about 3000, about 1500 to about 2500, about 1500 to about 2000, about 2000 to about 4000, about 2000 to about 3500, about 2000 to about 3000, about 2000 to about 2500, about 2500 to about 4000, about 2500 to about 3500, about 2500 to about 3000, about 3000 to about 4000, about 3000 to about 3500, or about 3500 to about 4000) nucleotides. In some embodiments, the length of the molecular barcode is sufficient to uniquely barcode the molecules and the length/sequence of the molecular barcode does not interfere with the downstream amplification steps.

In some embodiments, the molecular barcode sequence can be random. In some embodiments, the molecular barcode sequence can be a random N-mer. For example, if the molecular barcode sequence has a length of six nt, then it may be a random hexamer. If the molecular barcode sequence has a length of 12 nt, then it may be a random 12-mer.

In some embodiments, molecular barcodes can be made using random addition of nucleotides to form a sequence having a length to be used as an identifier. At each position of addition, a selection from one of four deoxyribonucleotides may be used. Alternatively a selection from one of three, two, or one deoxyribonucleotides may be used. Thus the molecular barcode may be fully random, somewhat random, or non-random in certain positions. In some embodiments, the molecular barcodes are not random N-mers, but are selected from a predetermined set of molecular barcode sequences. Exemplary molecular barcodes suitable for use in the methods disclosed herein are described in PCT/US2012/033207, which is hereby incorporated by reference in its entirety.

Attachment of a molecular barcode to a nucleic acid fragment may be performed by any means known in the art, including enzymatic, chemical, or biologic. In some embodiments, one means employs a polymerase chain reaction. In some embodiments, another means employs a ligase enzyme. For example, the ligase enzyme may be mammalian or bacterial. Other enzymes which may be used for attaching are other polymerase enzymes. A molecular barcode may be added to one or both ends of the fragments, preferably to both ends. In some embodiments, a molecular barcode may be contained within a nucleic acid molecule that contains other regions for other intended functionality. For example, a universal priming site may be added to permit later amplification. In some embodiments, another additional site may be a region of complementarity to a particular region or gene in the nucleic acid fragment.

### Initial Amplification of the Adapter-Attached Templates

Following adapter attachment, the adapter-attached templates can be amplified (e.g., PCR amplified) in an initial amplification reaction. Any appropriate method can be used to amplify the adapter-attached templates. An exemplary method that can be used to amplify the adapter-attached templates includes, without limitation, whole-genome PCR.

Any appropriate primer pair can be used to amplify the adapter-attached templates. In some cases, a universal primer pair can be used. A primer can include, without limitation from about 12 nucleotides to about 30 nucleotides. Examples of primer pairs that can be used to amplify the adapter-attached templates as described herein include, without limitation, those described in Example 4.

Any appropriate PCR conditions can be used in the initial amplification. PCR amplification can include a denaturing phase, an annealing phase, and an extension phase. Each phase of an amplification cycle can include any appropriate conditions. In some cases, a denaturing phase can include a temperature of about 90° C. to about 105° C. (e.g., about 94° C. to about 98° C.), and a time of about 1 second to about 5 minutes (e.g., about 10 seconds to about 1 minute). For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, annealing and extension phases can be performed in a single cycle. For example, an annealing and phase extension phase can include a temperature of about 65° C. for about 75 seconds.

PCR conditions used in the initial amplification can include any appropriate number of PCR amplification cycles. In some cases, PCR amplification can include from about 1 to about 50 cycles. In some embodiments, the PCR amplification comprises no more than 11 cycles. In some embodiments, the PCR amplification comprises no more than 7 cycles. In some embodiments, the PCR amplification comprises no more than 5 cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

In some cases, a duplex sequencing library generated as described herein (e.g., an amplified duplex sequencing library) can be purified. Any appropriate method can be used to purify a duplex sequencing library. An exemplary method that can be used to purify a duplex sequencing library includes, without limitation, magnetic beads (e.g., solid phase reversible immobilization (SPRI) magnetic beads).

Optional ssDNA Library Prep

In some cases, a duplex sequencing library can be used to generate a library of single stranded Watson strand-derived sequences and a library of single stranded Crick-strand derived sequences. Generating a library of single stranded Watson strand-derived sequences and a library of single stranded Crick-strand derived sequences can minimize non-specific amplification (e.g., from a primer complementary to a ligated sequence such as a 3′ duplex adapter or a 5′ adapter). Any appropriate method can be used to generate a library of single stranded Watson strand-derived sequences and a library of single stranded Crick-strand derived sequences (e.g., from a duplex sequencing library generated as described herein). In some cases, a library of single stranded Watson strand-derived sequences and a library of single stranded Crick-strand derived sequences can be generated from an amplified duplex sequencing library by dividing the amplification products into at least two aliquots, and subjecting each aliquot to a PCR amplification where the Watson strand is amplified from a first aliquot, and the Crick strand is amplified from a second aliquot. For example, a first aliquot of amplification products from an amplified duplex sequencing library can be subjected to a PCR amplification using a primer pair where a first primer is biotinylated and a second primer is non-biotinylated to generate a single stranded library of Watson strands, and a second aliquot of amplification products from an amplified duplex sequencing library can be subjected to a PCR amplification using a primer pair where a first primer is non-biotinylated and a second primer is biotinylated to generate a single stranded library of Crick strands. In some cases, a library of single stranded Watson strand-derived sequences and a library of single stranded Crick-strand derived sequences can be generated.

Any appropriate method can be used to generate a library of single stranded Watson strand-derived sequences and a library of single stranded Crick-strand derived sequences from an amplified duplex sequencing library. For example, amplification products from an amplified duplex sequencing library can be separated into a first PCR amplification and a second PCR amplification in which only one of the two primers in the PCR primer pair is tagged. For example, a first PCR amplification can use a primer pair that includes a primer (e.g., a first primer) that is tagged and a primer (e.g., a second primer) that is not tagged, and a second PCR amplification can use a primer pair that includes a primer (e.g., a first primer) that is not tagged and a primer (e.g., a second primer) that is tagged. A primer tag can be any tag that enables a PCR amplification product generated from the tagged primer to be recovered. In some cases, a tagged primer can be a biotinylated primer, and a PCR amplification produce generated from the biotinylated primer can be recovered using streptavidin. For example, a library of single stranded Watson strand-derived sequences and a library of single stranded Crick-strand derived sequences can be generated in a PCR amplification using a primer pair including a biotinylated primer and a non-biotinylated primer. In some cases, a tagged primer can be a phosphorylated primer, and a PCR amplification produce generated from the phosphorylated primer can be recovered using a lambda nuclease. For example, a library of single stranded Watson strand-derived sequences and a library of single stranded Crick-strand derived sequences can be generated in a PCR amplification using a primer pair including a phosphorylated primer and a non-phosphorylated primer.

Any appropriate primer pair can be used to generate a library of single stranded Watson strand-derived sequences and a library of single stranded Crick-strand derived sequences (e.g., from a duplex sequencing library generated as described herein). A primer can include, without limitation, from about 12 nucleotides to about 30 nucleotides. In some cases, a primer pair can include at least one primer that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification). Examples of primer pairs that can be used to generate a library of single stranded Watson strand-derived sequences and a library of single stranded Crick-strand derived sequences as described herein include, without limitation, a P5 primer and a P7 primer.

Any appropriate PCR conditions can be used to generate a library of single stranded Watson strand-derived sequences and a library of single stranded Crick-strand derived sequences (e.g., from a duplex sequencing library generated as described herein). PCR amplification can include a denaturing phase, an annealing phase, and an extension phase. Each phase of an amplification cycle can include any appropriate conditions. In some cases, a denaturing phase can include a temperature of about 90° C. to about 105° C., and a time of about 1 second to about 5 minutes. For example, a denaturing phase can include a temperature of about 98° C. for about 10 seconds. In some cases, an annealing phase can include a temperature of about 50° C. to about 72° C., and a time of about 30 seconds to about 90 seconds. In some cases, an extension phase can include a temperature of about 55° C. to about 80° C., and a time of about 15 seconds per kb of the amplicon to be generated to about 30 seconds per kb of the amplicon to be generated. In some cases, an extension phase reflects the processivity of the polymerase that is used. In some cases, annealing and extension phases can be performed in a single cycle. For example, an annealing and phase extension phase can include a temperature of about 65° C. for about 75 seconds.

PCR conditions used to generate a library of single stranded Watson strand-derived sequences and a library of single stranded Crick-strand derived sequences (e.g., from a duplex sequencing library generated as described herein) can include any appropriate number of PCR amplification cycles. In some cases, PCR amplification can include, without limitation, from about 1 to about 50 cycles. For example, PCR amplification can include about 4 amplification cycles.

In some cases, when PCR conditions include a heat-activated polymerase, PCR amplification also can include an initialization step. For example, PCR amplification can include an initialization step prior to performing the PCR amplification cycles. In some cases, an initialization step can include a temperature of about 94° C. to about 98° C., and a time of about 15 seconds to about 1 minute. For example, an initialization step can include a temperature of about 98° C. for about 30 seconds.

In some cases, PCR amplification also can include a hold step. For example, PCR amplification can include a hold step after performing the PCR amplification cycles, an optionally after performing any final extension step. In some case, a hold step can include a temperature of about 4° C. to about 15° C., for an indefinite amount of time.

Any appropriate method can be used to separate double stranded amplification products into single stranded amplification products. In some cases, a double stranded amplification products can be denatured to separate double stranded amplification products into two single stranded amplification products. Examples of methods that can be used to separate a double stranded amplification product into single stranded amplification products include, without limitation, heat denaturation, chemical (e.g., NaOH) denaturation, and salt denaturation.

Following PCR amplification, the tagged PCR amplification products can be recovered. Any appropriate method can be used to recover tagged PCR amplification products generated using a tagged primer. In cases where a tagged primer is a biotinylated primer, the biotinylated amplification products (e.g., generated from the biotinylated primer) can be recovered using streptavidin (e.g., streptavidin-functionalized beads). For example, when an amplified duplex sequencing library is further amplified in a first PCR amplification using a primer pair that includes a first biotinylated primer and a second non-biotinylated primer, and a second PCR amplification using a primer pair that includes a first non-biotinylated primer and a second biotinylated primer, the biotinylated amplification products generated from the first PCR amplification can be bound to streptavidin-functionalized beads (e.g., a first set of streptavidin-functionalized beads) and the biotinylated amplification products generated from the second PCR amplification can be bound to streptavidin-functionalized beads (e.g., a first second of streptavidin-functionalized beads), and the double stranded amplification products can be separated (e.g., denatured) into single strands of the amplification products. In some cases, recovering biotinylated PCR amplification products also can include releasing the biotinylated PCR amplification products from the streptavidin (e.g., the streptavidin-functionalized beads). Separating the double stranded amplification products generated by a first PCR amplification using a primer pair that includes a first biotinylated primer and a second non-biotinylated primer, and a second PCR amplification using a primer pair that includes a first non-biotinylated primer and a second biotinylated primer, can allow single stranded amplification products generated from the biotinylated primers to remain bound to the streptavidin-functionalized beads while single stranded amplification products generated from the non-biotinylated primers can be denatured (e.g., denatured and degraded) from the streptavidin-functionalized beads, thereby generating a library of single stranded Watson strand-derived sequences and a library of single stranded Crick-strand derived sequences of the duplex sequencing library.

In cases where a tagged primer is a phosphorylated primer, the phosphorylated amplification products (e.g., generated from the phosphorylated primer) can be recovered using an exonuclease (e.g., a lambda exonuclease). For example, when an amplified duplex sequencing library is further amplified in a first PCR amplification using a primer pair that includes a first phosphorylated primer and a second non-phosphorylated primer, and a second PCR amplification using a primer pair that includes a first non-phosphorylated primer and a second phosphorylated primer, the double stranded amplification products can be separated into single strands of the amplification products. Separating the double stranded amplification products generated by a first PCR amplification using a primer pair that includes a first phosphorylated primer and a second non-phosphorylated primer, and a second PCR amplification using a primer pair that includes a first non-phosphorylated primer and a second phosphorylated primer, can allow single stranded amplification products generated from the non-phosphorylated primers to be recovered while single stranded amplification products generated from the phosphorylated primers can be degraded by a lambda exonuclease, thereby generating a library of single stranded Watson strand-derived sequences and a library of single stranded Crick-strand derived sequences of the duplex sequencing library.

### Target Enrichment

In some embodiments of any one of the methods herein, amplicons produced by the initial amplification are enriched for one or more target polynucleotides. In some embodiments, prior to target enrichment, single-stranded DNA libraries are prepared from amplicons produced by the initial amplification. Exemplary methods for producing the single-stranded DNA libraries are described herein.

Any appropriate method can be used to amplify a target region from a library of amplification products (e.g., a duplex sequencing library, a library of single stranded Watson strand-derived sequences, or a library of single stranded Crick-strand derived sequences generated as described herein). In some cases, a target region can be amplified from library of amplification products by subjecting the library of amplification products to a PCR amplification using a primer pair where a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest).

In some cases, a target region can be amplified from a library of amplification products (e.g., a duplex sequencing library, a library of single stranded Watson strand-derived sequences, or a library of single stranded Crick-strand derived sequences generated as described herein) in a single PCR amplification. For example, a target region can be amplified from a library of amplification products in a single PCR amplification using a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region.

In some cases, a target region can be amplified from a library of amplification products (e.g., a duplex sequencing library, a library of single stranded Watson strand-derived sequences, or a library of single stranded Crick-strand derived sequences generated as described herein) in multiple PCR amplifications. Multiple PCR amplifications (e.g., a first PCR amplification and a subsequent, nested PCR amplification) can be used to increase the specificity of amplifying a target region. For example, a target region can be amplified from a library of amplification products in a series of PCR amplifications where a first PCR amplification uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a target region, and subjecting the amplification products generated in the first PCR amplification to a subsequent, nested PCR amplification that uses a primer pair including a first primer that can target an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a second primer that can target a nucleic acid sequence from the target region that is present in the amplification products generated in the first PCR amplification.

Any appropriate primer pair can be used to amplify a target region from a library of amplification products (e.g., a duplex sequencing library, a library of single stranded Watson strand-derived sequences, or a library of single stranded Crick-strand derived sequences generated as described herein). A primer can include, without limitation, from about 12 nucleotides to about 30 nucleotides. In some cases, a primer pair can include a primer (e.g., a first primer) that can target (e.g., target and bind to) an adapter sequence (e.g., an adapter sequence containing a molecular barcode) present in an amplification product generated as described herein (e.g., by ligating a 3′ duplex adapter including a first molecular barcode and a 5′ adapter including a second molecular barcode to a nucleic acid fragment in a duplex sequencing library prior to the amplification) and a primer (e.g., a second primer) that can target (e.g., target and bind to) a target region (e.g., a region of interest). Examples of primers that can target an adapter sequence containing a molecular barcode present in an amplification product generated as described herein (e.g., by ligating a 3′ duplex adapter including a first molecular barcode and a 5′ adapter including a second molecular barcode to a nucleic acid fragment in a duplex sequencing library prior to the amplification) include, without limitation, an i5 index primer and an i7 index primer. Primers that can target a target region can include a sequence that is complementary to the target region.

In some embodiments, in cases where a target region is a nucleic acid encoding an immune cell receptor, a primer that can target the target region includes a sequence that is complementary to the sequence of the immune cell receptor. In some embodiments, in cases where a target region is a nucleic acid encoding a T cell receptor, a primer that can target the target region includes a sequence that is complementary to the sequence of the T cell receptor. In some embodiments, in cases where a target region is a nucleic acid encoding a T cell receptor, a primer that can target the target region can include a nucleic acid sequence of SEQ ID NO: 4 (e.g., a nucleic acid sequence comprising SEQ ID NO: 4). In some embodiments, in cases where a target region is a nucleic acid encoding a T cell receptor, a primer that can target the target region can include a nucleic acid sequence of SEQ ID NO: 5 (e.g., a nucleic acid sequence comprising SEQ ID NO: 5). In some embodiments, in cases where a target region is a nucleic acid encoding a T cell receptor, a primer that can target the target region can include a nucleic acid sequence of SEQ ID NO: 6 (e.g., a nucleic acid sequence comprising SEQ ID NO: 6). In some embodiments, in cases where a target region is a nucleic acid encoding a T cell receptor, a primer that can target the target region can include a nucleic acid sequence of SEQ ID NO: 7 (e.g., a nucleic acid sequence comprising SEQ ID NO: 7). In some embodiments, in cases where a target region is a nucleic acid encoding a T cell receptor, a primer that can target the target region can include a nucleic acid sequence of SEQ ID NO: 8 (e.g., a nucleic acid sequence comprising SEQ ID NO: 8). In some embodiments, in cases where a target region is a nucleic acid encoding a T cell receptor, a primer that can target the target region can include a nucleic acid sequence of SEQ ID NO: 9 (e.g., a nucleic acid sequence comprising SEQ ID NO: 9). In some embodiments, in cases where a target region is a nucleic acid encoding a T cell receptor, a primer that can target the target region can include a nucleic acid sequence of SEQ ID NO: 10 (e.g., a nucleic acid sequence comprising SEQ ID NO: 10). In some embodiments, in cases where a target region is a nucleic acid encoding a T cell receptor, a primer that can target the target region can include a nucleic acid sequence of SEQ ID NO: 11 (e.g., a nucleic acid sequence comprising SEQ ID NO: 11). In some embodiments, in cases where a target region is a nucleic acid encoding a T cell receptor, a primer that can target the target region can include a nucleic acid sequence of SEQ ID NO: 12 (e.g., a nucleic acid sequence comprising SEQ ID NO: 12). In some embodiments, in cases where a target region is a nucleic acid encoding a T cell receptor, a primer that can target the target region can include a nucleic acid sequence of SEQ ID NO: 13 (e.g., a nucleic acid sequence comprising SEQ ID NO: 13). In some embodiments, in cases where a target region is a nucleic acid encoding a T cell receptor, a primer that can target the target region can include a nucleic acid sequence of SEQ ID NO: 14 (e.g., a nucleic acid sequence comprising SEQ ID NO: 14). In some embodiments, in cases where a target region is a nucleic acid encoding a T cell receptor, a primer that can target the target region can include a nucleic acid sequence of SEQ ID NO: 15 (e.g., a nucleic acid sequence comprising SEQ ID NO: 15). In some embodiments, in cases where a target region is a nucleic acid encoding a T cell receptor, a primer that can target the target region can include a nucleic acid sequence of SEQ ID NO: 16 (e.g., a nucleic acid sequence comprising SEQ ID NO: 16). In some embodiments, in cases where a target region is a nucleic acid encoding a T cell receptor, a primer that can target the target region can include a nucleic acid sequence of SEQ ID NO: 17 (e.g., a nucleic acid sequence comprising SEQ ID NO: 17). In some embodiments, in cases where a target region is a nucleic acid encoding a T cell receptor, a primer that can target the target region can include a nucleic acid sequence of SEQ ID NO: 18 (e.g., a nucleic acid sequence comprising SEQ ID NO: 18). In some embodiments, in cases where a target region is a nucleic acid encoding a T cell receptor, a primer that can target the target region can include a nucleic acid sequence of SEQ ID NO: 19 (e.g., a nucleic acid sequence comprising SEQ ID NO: 19). In some embodiments, in cases where a target region is a nucleic acid encoding a T cell receptor, a primer that can target the target region can include a nucleic acid sequence of SEQ ID NO: 20 (e.g., a nucleic acid sequence comprising SEQ ID NO: 20). In some embodiments, in cases where a target region is a nucleic acid encoding a T cell receptor, a primer that can target the target region can include a nucleic acid sequence of SEQ ID NO: 21 (e.g., a nucleic acid sequence comprising SEQ ID NO: 21). In some embodiments, in cases where a target region is a nucleic acid encoding a T cell receptor, a primer that can target the target region can include a nucleic acid sequence of SEQ ID NO: 22 (e.g., a nucleic acid sequence comprising SEQ ID NO: 22). In some embodiments, in cases where a target region is a nucleic acid encoding a T cell receptor, a primer that can target the target region can include a nucleic acid sequence of SEQ ID NO: 23 (e.g., a nucleic acid sequence comprising SEQ ID NO: 23). In some embodiments, in cases where a target region is a nucleic acid encoding a T cell receptor, a primer that can target the target region can include a nucleic acid sequence of SEQ ID NO: 24 (e.g., a nucleic acid sequence comprising SEQ ID NO: 24). In some embodiments, in cases where a target region is a nucleic acid encoding a T cell receptor, a primer that can target the target region can include a nucleic acid sequence of SEQ ID NO: 25 (e.g., a nucleic acid sequence comprising SEQ ID NO: 25). In some embodiments, in cases where a target region is a nucleic acid encoding a T cell receptor, a primer that can target the target region can include a nucleic acid sequence of SEQ ID NO: 26 (e.g., a nucleic acid sequence comprising SEQ ID NO: 26). In some embodiments, in cases where a target region is a nucleic acid encoding a T cell receptor, a primer that can target the target region can include a nucleic acid sequence of SEQ ID NO: 27 (e.g., a nucleic acid sequence comprising SEQ ID NO: 27). In some embodiments, in cases where a target region is a nucleic acid encoding a T cell receptor, a primer that can target the target region can include a nucleic acid sequence of SEQ ID NO: 28 (e.g., a nucleic acid sequence comprising SEQ ID NO: 28). In some embodiments, in cases where a target region is a nucleic acid encoding a T cell receptor, a primer that can target the target region can include a nucleic acid sequence of SEQ ID NO: 29 (e.g., a nucleic acid sequence comprising SEQ ID NO: 29). In some embodiments, in cases where a target region is a nucleic acid encoding a T cell receptor, a primer that can target the target region can include a nucleic acid sequence of SEQ ID NO: 30 (e.g., a nucleic acid sequence comprising SEQ ID NO: 30). In some embodiments, in cases where a target region is a nucleic acid encoding a T cell receptor, a primer that can target the target region can include a nucleic acid sequence of SEQ ID NO: 31 (e.g., a nucleic acid sequence comprising SEQ ID NO: 31). In some embodiments, in cases where a target region is a nucleic acid encoding a T cell receptor, a primer that can target the target region can include a nucleic acid sequence of SEQ ID NO: 32 (e.g., a nucleic acid sequence comprising SEQ ID NO: 32). In some embodiments, in cases where a target region is a nucleic acid encoding a T cell receptor, a primer that can target the target region can include a nucleic acid sequence of SEQ ID NO: 33 (e.g., a nucleic acid sequence comprising SEQ ID NO: 33). In some embodiments, in cases where a target region is a nucleic acid encoding a T cell receptor, a primer that can target the target region can include a nucleic acid sequence of SEQ ID NO: 34 (e.g., a nucleic acid sequence comprising SEQ ID NO: 34). In some embodiments, in cases where a target region is a nucleic acid encoding a T cell receptor, a primer that can target the target region can include a nucleic acid sequence of SEQ ID NO: 35 (e.g., a nucleic acid sequence comprising SEQ ID NO: 35). In some embodiments, in cases where a target region is a nucleic acid encoding a T cell receptor, a primer that can target the target region can include a nucleic acid sequence of SEQ ID NO: 36 (e.g., a nucleic acid sequence comprising SEQ ID NO: 36). In some embodiments, in cases where a target region is a nucleic acid encoding a T cell receptor, a primer that can target the target region can include a nucleic acid sequence of SEQ ID NO: 37 (e.g., a nucleic acid sequence comprising SEQ ID NO: 37). In some embodiments, in cases where a target region is a nucleic acid encoding a T cell receptor, a primer that can target the target region can include a nucleic acid sequence of SEQ ID NO: 38 (e.g., a nucleic acid sequence comprising SEQ ID NO: 38). In some embodiments, in cases where a target region is a nucleic acid encoding a T cell receptor, a primer that can target the target region can include a nucleic acid sequence of SEQ ID NO: 39 (e.g., a nucleic acid sequence comprising SEQ ID NO: 39). In some embodiments, in cases where a target region is a nucleic acid encoding a T cell receptor, a primer that can target the target region can include a nucleic acid sequence of SEQ ID NO: 40 (e.g., a nucleic acid sequence comprising SEQ ID NO: 40). In some embodiments, in cases where a target region is a nucleic acid encoding a T cell receptor, a primer that can target the target region can include a nucleic acid sequence of SEQ ID NO: 41 (e.g., a nucleic acid sequence comprising SEQ ID NO: 41). In some embodiments, in cases where a target region is a nucleic acid encoding a T cell receptor, a primer that can target the target region can include a nucleic acid sequence of SEQ ID NO: 42 (e.g., a nucleic acid sequence comprising SEQ ID NO: 42). In some embodiments, in cases where a target region is a nucleic acid encoding a T cell receptor, a primer that can target the target region can include a nucleic acid sequence of SEQ ID NO: 43 (e.g., a nucleic acid sequence comprising SEQ ID NO: 43). In some embodiments, in cases where a target region is a nucleic acid encoding a T cell receptor, a primer that can target the target region can include a nucleic acid sequence of SEQ ID NO: 44 (e.g., a nucleic acid sequence comprising SEQ ID NO: 44). In some embodiments, in cases where a target region is a nucleic acid encoding a T cell receptor, a primer that can target the target region can include a nucleic acid sequence of SEQ ID NO: 45 (e.g., a nucleic acid sequence comprising SEQ ID NO: 45). In some embodiments, in cases where a target region is a nucleic acid encoding a T cell receptor, a primer that can target the target region can include a nucleic acid sequence of SEQ ID NO: 46 (e.g., a nucleic acid sequence comprising SEQ ID NO: 46). In some embodiments, in cases where a target region is a nucleic acid encoding a T cell receptor, a primer that can target the target region can include a nucleic acid sequence of SEQ ID NO: 47 (e.g., a nucleic acid sequence comprising SEQ ID NO: 47). In some embodiments, in cases where a target region is a nucleic acid encoding a T cell receptor, a primer that can target the target region can include a nucleic acid sequence of SEQ ID NO: 48 (e.g., a nucleic acid sequence comprising SEQ ID NO: 48). In some embodiments, in cases where a target region is a nucleic acid encoding a T cell receptor, a primer that can target the target region can include a nucleic acid sequence of SEQ ID NO: 49 (e.g., a nucleic acid sequence comprising SEQ ID NO: 49). In some embodiments, in cases where a target region is a nucleic acid encoding a T cell receptor, a primer that can target the target region can include a nucleic acid sequence of SEQ ID NO: 50 (e.g., a nucleic acid sequence comprising SEQ ID NO: 50). In some embodiments, in cases where a target region is a nucleic acid encoding a T cell receptor, a primer that can target the target region can include a nucleic acid sequence of SEQ ID NO: 51 (e.g., a nucleic acid sequence comprising SEQ ID NO: 51). In some embodiments, in cases where a target region is a nucleic acid encoding a T cell receptor, a primer that can target the target region can include a nucleic acid sequence of SEQ ID NO: 52 (e.g., a nucleic acid sequence comprising SEQ ID NO: 52). In some embodiments, in cases where a target region is a nucleic acid encoding a T cell receptor, a primer that can target the target region can include a nucleic acid sequence of SEQ ID NO: 53 (e.g., a nucleic acid sequence comprising SEQ ID NO: 53). In some embodiments, in cases where a target region is a nucleic acid encoding a T cell receptor, a primer that can target the target region can include a nucleic acid sequence of SEQ ID NO: 54 (e.g., a nucleic acid sequence comprising SEQ ID NO: 54). In some embodiments, in cases where a target region is a nucleic acid encoding a T cell receptor, a primer that can target the target region can include a nucleic acid sequence of SEQ ID NO: 55 (e.g., a nucleic acid sequence comprising SEQ ID NO: 55). In some embodiments, in cases where a target region is a nucleic acid encoding a T cell receptor, a primer that can target the target region can include a nucleic acid sequence of SEQ ID NO: 56 (e.g., a nucleic acid sequence comprising SEQ ID NO: 56). In some embodiments, in cases where a target region is a nucleic acid encoding a T cell receptor, a primer that can target the target region can include a nucleic acid sequence of SEQ ID NO: 57 (e.g., a nucleic acid sequence comprising SEQ ID NO: 57). In some embodiments, in cases where a target region is a nucleic acid encoding a T cell receptor, a primer that can target the target region can include a nucleic acid sequence of SEQ ID NO: 58 (e.g., a nucleic acid sequence comprising SEQ ID NO: 58). In some embodiments, in cases where a target region is a nucleic acid encoding a T cell receptor, a primer that can target the target region can include a nucleic acid sequence of SEQ ID NO: 59 (e.g., a nucleic acid sequence comprising SEQ ID NO: 59). In some embodiments, in cases where a target region is a nucleic acid encoding a T cell receptor, a primer that can target the target region can include a nucleic acid sequence of SEQ ID NO: 60 (e.g., a nucleic acid sequence comprising SEQ ID NO: 60). In some embodiments, in cases where a target region is a nucleic acid encoding a T cell receptor, a primer that can target the target region can include a nucleic acid sequence of SEQ ID NO: 61 (e.g., a nucleic acid sequence comprising SEQ ID NO: 61). In some embodiments, in cases where a target region is a nucleic acid encoding a T cell receptor, a primer that can target the target region can include a nucleic acid sequence of SEQ ID NO: 62 (e.g., a nucleic acid sequence comprising SEQ ID NO: 62). In some embodiments, in cases where a target region is a nucleic acid encoding a T cell receptor, a primer that can target the target region can include a nucleic acid sequence of SEQ ID NO: 63 (e.g., a nucleic acid sequence comprising SEQ ID NO: 63). In some embodiments, in cases where a target region is a nucleic acid encoding a T cell receptor, a primer that can target the target region can include a nucleic acid sequence of SEQ ID NO: 64 (e.g., a nucleic acid sequence comprising SEQ ID NO: 64). In some embodiments, in cases where a target region is a nucleic acid encoding a T cell receptor, a primer that can target the target region can include a nucleic acid sequence of SEQ ID NO: 65 (e.g., a nucleic acid sequence comprising SEQ ID NO: 65). In some embodiments, in cases where a target region is a nucleic acid encoding a T cell receptor, a primer that can target the target region can include a nucleic acid sequence of SEQ ID NO: 66 (e.g., a nucleic acid sequence comprising SEQ ID NO: 66). In some embodiments, in cases where a target region is a nucleic acid encoding a T cell receptor, a primer that can target the target region can include a nucleic acid sequence of SEQ ID NO: 67 (e.g., a nucleic acid sequence comprising SEQ ID NO: 67). In some embodiments, in cases where a target region is a nucleic acid encoding a T cell receptor, a primer that can target the target region can include a nucleic acid sequence of SEQ ID NO: 68 (e.g., a nucleic acid sequence comprising SEQ ID NO: 68).

TABLE 1

| SEQ ID NO. | Primer Sequence |
|---|---|
| SEQ ID NO: 4 | CTACAACTGTGAGTCTGGT |
| SEQ ID NO: 5 | GCCTTACCTACAACGGTTA |
| SEQ ID NO: 6 | CCTACAACAGTGAGCCAAC |
| SEQ ID NO: 7 | AAGACAGAGAGCTGGGT |
| SEQ ID NO: 8 | CCTAGGATGGAGAGTCGAG |
| SEQ ID NO: 9 | GTCACAGTGAGCCTGGT |
| SEQ ID NO: 10 | TACCTAGCACGGTGAGC |
| SEQ ID NO: 11 | CAGTACGGTCAGCCTAGA |
| SEQ ID NO: 12 | TACCGAGCACTGTCAGC |
| SEQ ID NO: 13 | CTTACCCAGCACTGAGAG |
| SEQ ID NO: 14 | CACCGAGCACCAGGAG |
| SEQ ID NO: 15 | CACCCAGCACGGTCAG |
| SEQ ID NO: 16 | CTGTGACCGTGAGCCT |
| SEQ ID NO: 17 | CCTGAAAAATGTCTTACCTACAACTG |
| SEQ ID NO: 18 | ACCCCCAGCCTTACCT |
| SEQ ID NO: 19 | GCCTTGACTTACTCACCTACAA |
| SEQ ID NO: 20 | GAAGTCTTTTACATACCCAAGACAG |
| SEQ ID NO: 21 | CCCTGATTCTGCAACTTACCTA |
| SEQ ID NO: 22 | CCCCCATACCTGTCACAG |
| SEQ ID NO: 23 | GAGCCCCCTTCTTACCTAG |
| SEQ ID NO: 24 | GCCTCCTTACCCAGTACG |
| SEQ ID NO: 25 | CCCGCTTACCGAGCAC |
| SEQ ID NO: 26 | GGCCCCAGCTTACCCA |
| SEQ ID NO: 27 | GCCCGCGCTCACCGAG |
| SEQ ID NO: 28 | CCGCGAAAACTCACCCA |
| SEQ ID NO: 29 | GCCCGAATCTCACCTGT |
| SEQ ID NO: 30 | AATGTCTTACCTACAACTGTGA |
| SEQ ID NO: 31 | CCCAGCCTTACCTACAAC |
| SEQ ID NO: 32 | CCTTGACTTACTCACCTACAACAG |
| SEQ ID NO: 33 | TACATACCCAAGACAGAGAGC |
| SEQ ID NO: 34 | GCAACTTACCTAGGATGGAGAG |
| SEQ ID NO: 35 | CCATACCTGTCACAGTGAGC |
| SEQ ID NO: 36 | CCCTTCTTACCTAGCACGG |
| SEQ ID NO: 37 | CTTACCCAGTACGGTCAGC |
| SEQ ID NO: 38 | CCGCTTACCGAGCACTG |
| SEQ ID NO: 39 | CCAGCTTACCCAGCACT |
| SEQ ID NO: 40 | CGCTCACCGAGCACCA |
| SEQ ID NO: 41 | GAAAACTCACCCAGCACG |
| SEQ ID NO: 42 | GAATCTCACCTGTGACCGT |
| SEQ ID NO: 43 | GCAAAAGAACCTGAAAAATGTCTTA |
| SEQ ID NO: 44 | CCCTCCTAGAGACCCCC |
| SEQ ID NO: 45 | CTGTCCAGCCTTGACTTACTC |
| SEQ ID NO: 46 | CTATCCCGAAAGAAGTCTTTTACATA |
| SEQ ID NO: 47 | CCACCCTGATTCTGCAACTT |
| SEQ ID NO: 48 | AAGAGTGGAGCCCCCATA |
| SEQ ID NO: 49 | CCACCTGGAGCCCCCT |
| SEQ ID NO: 50 | CCCCAACCGCCTCCTTA |
| SEQ ID NO: 51 | TTCAGCGGGAGCCCCC |
| SEQ ID NO: 52 | CGGCGGCCCCAGCTTA |
| SEQ ID NO: 53 | CAGCAGCCCGCGCTCA |
| SEQ ID NO: 54 | GGGTGGTCCCGCGAAA |
| SEQ ID NO: 55 | GGAGACGCCCGAATCTCA |
| SEQ ID NO: 56 | CTGAAAAATGTCTTACCTACAACTG |
| SEQ ID NO: 57 | GACCCCCAGCCTTACCT |
| SEQ ID NO: 58 | CCTTGACTTACTCACCTACAACA |
| SEQ ID NO: 59 | GAAGTCTTTTACATACCCAAGACAG |
| SEQ ID NO: 60 | TGATTCTGCAACTTACCTAGGATG |
| SEQ ID NO: 61 | CCCCCATACCTGTCACAG |
| SEQ ID NO: 62 | GAGCCCCCTTCTTACCTAG |
| SEQ ID NO: 63 | GCCTCCTTACCCAGTACGG |
| SEQ ID NO: 64 | GCCCCCGCTTACCGAG |
| SEQ ID NO: 65 | CCCAGCTTACCCAGCAC |
| SEQ ID NO: 66 | CGCGCTCACCGAGCAC |
| SEQ ID NO: 67 | CCGCGAAAACTCACCCA |
| SEQ ID NO: 68 | CGAATCTCACCTGTGACCG |

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more molecular barcodes.

In some cases, one or both primers of a primer pair used to amplify a target region from a library of amplification products (e.g., a duplex sequencing library, a library of single stranded Watson strand-derived sequences, or a library of single stranded Crick-strand derived sequences generated as described herein) can include one or more graft sequences (e.g. graft sequences for next generation sequencing).

In an aspect, the target enrichment comprises (a) selectively amplifying amplicons of Watson strands comprising the target polynucleotide sequence with a first set of Watson target-selective primer pairs, the first set of Watson target-selective primer pairs comprising: (i) a first Watson target-selective primer comprising a sequence complementary to the R2 sequencing primer site of the universal 3′ adapter sequence, and (ii) a second Watson target-selective primer comprising a target-selective sequence, thereby creating target Watson amplification products; and (b) selectively amplifying amplicons of Crick strands comprising the same target polynucleotide sequence with a first set of Crick target-selective primer pairs, the first set of Crick target-selective primer pairs comprising: (i) a first Crick target-selective primer comprising a sequence complementary to the R1 sequencing primer site of the universal 5′ adapter sequence, and (ii) a second Crick target-selective primer comprising the same target-selective sequence as the second Watson target-selective primer sequence, thereby creating target Crick amplification products.

In some embodiments, the method further comprises purifying the target Watson amplification products and the target Crick amplification products from non-target polynucleotides. In some embodiments, the purifying comprises attaching the target Watson amplification products and the target Crick amplification products to a solid support. In some embodiments, the first Watson target-selective primer and first Crick target-selective primer comprises a first member of an affinity binding pair, and wherein the solid support comprises a second member of the affinity binding pair. In some embodiments, the first member is biotin and the second member is streptavidin. In some embodiments, the solid support comprises a bead, well, membrane, tube, column, plate, sepharose, magnetic bead, or chip. In some embodiments, the method comprises removing polynucleotides that are not attached to the solid support.

In some embodiments, the method further comprises (a) further amplifying the target Watson amplification products with a second set of Watson target-selective primers, the second set of Watson target-selective primers comprising (i) a third Watson target-selective primer comprising a sequence complementary to the R2 sequencing primer site of the universal 3′ adapter sequence, and (ii) a fourth Watson target-selective primer comprising, in the 5′ to 3′ direction, an R1 sequencing primer site and a target-selective sequence selective for the same target polynucleotide, thereby creating target Watson library members; (b) further amplifying the target Crick amplification products with a second set of Crick target-selective primers, the second set of Crick target-selective primers comprising (i) a third Crick target-selective primer comprising a sequence complementary to the R1 sequencing primer site of the universal 3′ adapter sequence, and (ii) a fourth Crick target-selective primer comprising, in the 5′ to 3′ direction, an R2 sequencing primer site and the target-selective sequence selective for the same target polynucleotide of the fourth Watson target-selective primer, thereby creating target Crick library members.

In some embodiments, the third Watson and Crick target-selective primers further comprise a sample barcode sequence. In some embodiments, the third Watson target-selective primer further comprises a first grafting sequence that enables hybridization to a first grafting primer on a sequencer and wherein the third Crick target-selective primer further comprises a second grafting sequence that enables hybridization to a second grafting primer on the sequencer. In some embodiments, the fourth Watson target-selective primer further comprises the second grafting sequence and wherein the fourth Crick target-selective primer further comprises the first grafting sequence. In some embodiments, the first grafting sequence is a P7 sequence and wherein the second grafting sequence is a P5 sequence.

Any appropriate PCR conditions can be used to generate an amplified target region as described herein (e.g., from a library of amplification products such as a duplex sequencing library, a library of single stranded Watson strand-derived sequences, or a library of single stranded Crick-strand derived sequences generated). Exemplary PCR conditions are described herein. PCR conditions used to generate an amplified target region as described herein (e.g., from a library of amplification products such as a duplex sequencing library, a library of single stranded Watson strand-derived sequences, or a library of single stranded Crick-strand derived sequences generated) can include any appropriate number of PCR amplification cycles. In some cases, PCR amplification can include, without limitation, from about 1 to about 50 cycles. For example, when PCR amplification of an amplified target region includes a single PCR amplification, the PCR amplification can include about 18 amplification cycles. For example, when PCR amplification of an amplified target region includes a first PCR amplification and a subsequent, nested PCR amplification, the first PCR amplification can include about 18 amplification cycles, and the subsequent, nested PCR amplification can include about 10 amplification cycles.

### Exemplary Targets

Any appropriate target region (e.g., a region of interest) can be amplified from a library of amplification products (e.g., a duplex sequencing library, a library of single stranded Watson strand-derived sequences, or a library of single stranded Crick-strand derived sequences generated as described herein) and assessed for TCR/BCR receptor sequences. In some cases, a target region can be a region of nucleic acid encoding an immune cell receptor. Examples of target regions that can be amplified and assessed for determining a sequence can include, but are not limited to, nucleic acid encoding a pattern recognition receptor (PRR), nucleic acid encoding a toll-like receptor (TLR), nucleic acid encoding a c-type lectin receptor (CLR), nucleic acid encoding a NOD-like receptor (NLR), nucleic acid encoding a RIG-I-like receptor, nucleic acid encoding a killer activated receptor (KAR), nucleic acid encoding a killer inhibitor receptor (KIR), nucleic acid encoding a complement receptor, nucleic acid encoding a Fc receptor, nucleic acid encoding a B cell receptor, nucleic acid encoding a T cell receptor, and nucleic acid encoding a cytokine receptor. In some embodiments, a target region that can be amplified and assessed can include a nucleic acid encoding a T cell receptor. In some embodiments, a target region that can be amplified and assessed can include a nucleic acid encoding a B cell receptor.

Any appropriate method can be used to assess a target region (e.g., an amplified target region) for determining TCR/BCR receptor sequences. In some cases, one or more sequencing methods can be used to assess an amplified target region for determining TCR/BCR receptor sequences.

### Sequence Determination

In some cases, one or more sequencing methods can be used to assess an amplified target region determine TCR/BCR receptor sequences. In some cases, sequencing reads can be used to assess an amplified target region for TCR/BCR receptor sequences and can be used to determine TCR/BCR receptor sequences by using both the Watson strand and the Crick strand. Examples of sequencing methods that can be used to assess an amplified target region for the TCR/BCR receptor sequences as describe herein include, without limitation, single read sequencing, paired-end sequencing, NGS, and deep sequencing. In some embodiments, the single read sequencing comprises sequencing across the entire length of the templates to generate the sequence reads. In some embodiments, the sequencing comprises paired end sequencing. In some embodiments, the sequencing is performed with a massively parallel sequencer. In some embodiments, the massively parallel sequencer is configured to determine sequence reads from both ends of template polynucleotides.

### Analysis of Sequence Reads

In some embodiments, the sequence reads are mapped to a reference genome.

In some embodiments, the sequence reads are assigned into UID families. A UID family can comprise sequence reads from amplicons originating from an original template, e.g., original double-stranded DNA fragment from a nucleic acid sample.

In some embodiments, each member of a UID family comprises the same exogenous UID sequence. In some embodiments, each member of a UID family further comprises the same endogenous UID sequence. Endogenous UIDs are described herein.

In some embodiments, each member of a UID family further comprises the same exogenous UID sequence and the same endogenous UID sequence. In some embodiments, the combination of the exogenous UID sequence and endogenous UID sequence are unique to the UID family. In some embodiments, the combination of the exogenous UID sequence and endogenous UID sequence does not exist in another UID family represented in the nucleic acid sample.

The number of members of a UID family can depend on the depth of sequencing. In some embodiments, a UID family comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or 1000 members. In some embodiments, a UID family comprises about 2-1000 members, about 2-500 members, about 2-100 members, about 2-50 members, or about 2-20 members.

In some embodiments, the sequence reads of an individual UID family are assigned to a Watson subfamily and a Crick subfamily. In some embodiments, the sequence reads of an individual UID family are assigned to the Watson and Crick subfamilies based on the orientation of the insert relative to the adapter sequences. In some embodiments, the orientation of the insert relative to the adapter sequences is resolved by how the sequence reads were aligned as “read pairs” or “mate pairs”.

In some embodiments, the assignment of the sequence reads into the Watson and Crick subfamilies are based on spatial relationship of the exogenous UID sequence to the R1 and R2 read sequence. In some embodiments, members of the Watson subfamily are characterized by the exogenous UID sequence being downstream of the R2 sequence and upstream of the R1 sequence. In some embodiments, members of the Crick subfamily are characterized by the exogenous UID sequence being downstream of the R1 sequence and upstream of the R2 sequence. In some embodiments, members of the Watson subfamily are characterized by the exogenous UID sequence being in greater proximity to the R2 sequence and lesser proximity to the R1 sequence. In some embodiments, members of the Crick subfamily are characterized by the exogenous UID sequence being in greater proximity to the R1 sequence and in lesser proximity to the R2 sequence. In some embodiments, members of the Watson subfamily are characterized by the exogenous UID sequence being immediately downstream or within 1-70, 1-60, 1-50, 1-40, 1-30, 1-20, 1-10, or 1-5 nucleotides of the R2 sequence. In some embodiments, members of the Crick subfamily are characterized by the exogenous UID sequence being immediately downstream or within 1-70, 1-60, 1-50, 1-40, 1-30, 1-20, 1-10, or 1-5 nucleotides of the R1 sequence.

In some embodiments, a UID subfamily (e.g., Watson subfamily and/or Crick subfamily) comprises at least 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 members. In some embodiments, a UID subfamily (e.g., Watson subfamily and/or Crick subfamily) comprises about 2-500 members, about 2-100 members, about 2-50 members, about 2-20 members, or about 2-10 members.

In some embodiments, a nucleotide sequence is determined to accurately represent a Watson strand of an analyte DNA fragment, e.g., a double stranded DNA fragment from the nucleic acid sample, when a threshold percentage (or a percentage exceeding a threshold) of members of the Watson subfamily contain the sequence. In some embodiments, a nucleotide sequence is determined to accurately represent a Crick strand of an analyte DNA fragment, e.g., a double stranded DNA fragment from the nucleic acid sample, when a threshold percentage (or a percentage exceeding a threshold) of members of the Crick subfamily contain the sequence.

Thresholds can be determined by a skilled artisan based on, e.g., number of the members of the subfamily, the particular purpose of the sequencing experiment, and the particular parameters of the sequencing experiment. In some embodiments, the threshold is set at 1%, 5%, 10%, 20%, 30%, 40%, 50%, 60%, 70%, 75%, 80%, 85%, 90%, 95%, 96%, 97%, 98%, 99%, or 100%. In particular embodiments, the threshold is set at 50%. By way of example only, in an embodiment wherein the threshold is set at 50%, a nucleotide sequence is determined to accurately represent a Watson or Crick strand of an analyte DNA fragment, e.g., a double stranded DNA fragment from the nucleic acid sample, when at least 50% of the subfamily members contain the sequence. By way of other example only, in an embodiment wherein the threshold is set at 50%, a nucleotide sequence is determined to accurately represent a Watson or

Crick strand of an analyte DNA fragment, e.g., a double stranded DNA fragment from the nucleic acid sample, when more than 50% of the subfamily members contain the sequence.

In some embodiments, the sequence accurately representing the Watson strand of the analyte DNA fragment is determined to include a TCR/BCR receptor sequence.

In some embodiments, the sequence accurately representing the Crick strand of the analyte DNA fragment is determined to include a TCR/BCR receptor sequence.

In some embodiments, the analyte DNA fragment is used to determine the TCR/BCR receptor sequences when the sequence accurately representing the Watson strand the sequence accurately representing the Crick strand comprise the same sequence.

In some cases, the location of the molecular barcode within the paired-end sequencing reads of the amplified target region can be used to distinguish which strand of the double stranded nucleic acid template the amplified target region was derived from. For example, when a first a paired-end sequencing read of an amplified target region indicates that a molecular barcode is read last, the amplified target region can be identified as being derived from the sense strand of the nucleic acid template, and when a first a paired-end sequencing read of an amplified target region indicates that a molecular barcode is read first, the amplified target region can be identified as being derived from the anti-sense strand of the nucleic acid template. For example, when a second a paired-end sequencing read of an amplified target region indicates that a molecular barcode is read first, the amplified target region can be identified as being derived from the anti-sense strand of the nucleic acid template, and when a second a paired-end sequencing read of an amplified target region indicates that a molecular barcode is read last, the amplified target region can be identified as being derived from the sense strand of the nucleic acid template. In some cases, paired-end sequencing can be used to distinguish amplification products derived from the Watson strand from amplification products derived from the Crick strand.

Following sequencing of target regions (e.g., target regions amplified as described herein), sequencing reads can be aligned to a reference genome and grouped by the molecular barcode present in each sequencing read. In some cases, sequencing reads that include the same molecular barcode and map to both the Watson strand and the Crick strand of the double stranded nucleic acid template (e.g., both the Watson strand and the Crick strand of the target region) can be identified as having duplex support. For example, when sequencing reads indicate the presence of one or more mutations in a target region include the same molecular barcode and map to both the Watson strand and the Crick strand of the target region, the mutation(s) can be identified as having duplex support.

### Immune Cell Receptors

As used herein, an “immune cell receptor” refers a receptor, usually on a cell membrane, which binds to a substance (e.g., a cytokine) and causes a response in the immune system. For example, immune cell receptors in the immune system can include, but are not limited to, pattern recognition receptors (PRRs), toll-like receptors (TLRs), killer activated and killer inhibitor receptors (KARs and KIRs), complement receptors, Fc receptors, B cell receptors and T cell receptors. In some embodiments, an immune cell receptor is a T cell receptor. In some embodiments, an immune cell receptor is a B cell receptor.

A T cell receptor (TCR) is a protein complex found on the surface of T cells, or T lymphocytes, that is responsible for recognizing fragments of antigen as peptides bound to major histocompatibility complex (MHC) molecules. The generation of TCR diversity is similar to that for antibodies and B-cell antigen receptors. In some embodiments, it arises mainly from genetic recombination of the DNA-encoded segments in individual somatic T cells by somatic V(D)J recombination using RAG1 and RAG2 recombinases. Unlike immunoglobulins, however, TCR genes do not undergo somatic hypermutation, and T cells do not express activation-induced cytidine deaminase (AID). The recombination process that creates diversity in BCR (e.g., antibodies) and TCR is unique to lymphocytes (T and B cells) during the early stages of their development in primary lymphoid organs (thymus for T cells, bone marrow for B cells).

A B cell receptor (BCR) is a transmembrane protein on the surface of a B cell. A B cell receptor is composed of a membrane-bound immunoglobulin molecule and a signal transduction moiety. The former forms a type 1 transmembrane receptor protein, and is typically located on the outer surface of these lymphocyte cells. Through biochemical signaling and by physically acquiring antigens from the immune synapses, the BCR controls the activation of the B cell. Within the BCR, the part that recognizes antigens is composed of three distinct genetic regions, referred to as V, D, and J. All these regions are recombined and spliced at the genetic level in a combinatorial process that is exceptional to the immune system. There are a number of genes that encode each of these regions in the genome and can be joined in various ways to generate a wide range of receptor molecules. The production of this variety is crucial since the body may encounter many more antigens than the available genes. Through this process, the body finds a way of producing multiple different combinations of antigen-recognizing receptor molecules. Heavy chain rearrangement of the BCR entails the initial steps in the development of B cell.

### EXAMPLES

The disclosure is further described in the following examples, which do not limit the scope of the disclosure described in the claims.

### Example 1—PCR-Based Enrichment of BCR and TCR Sequences

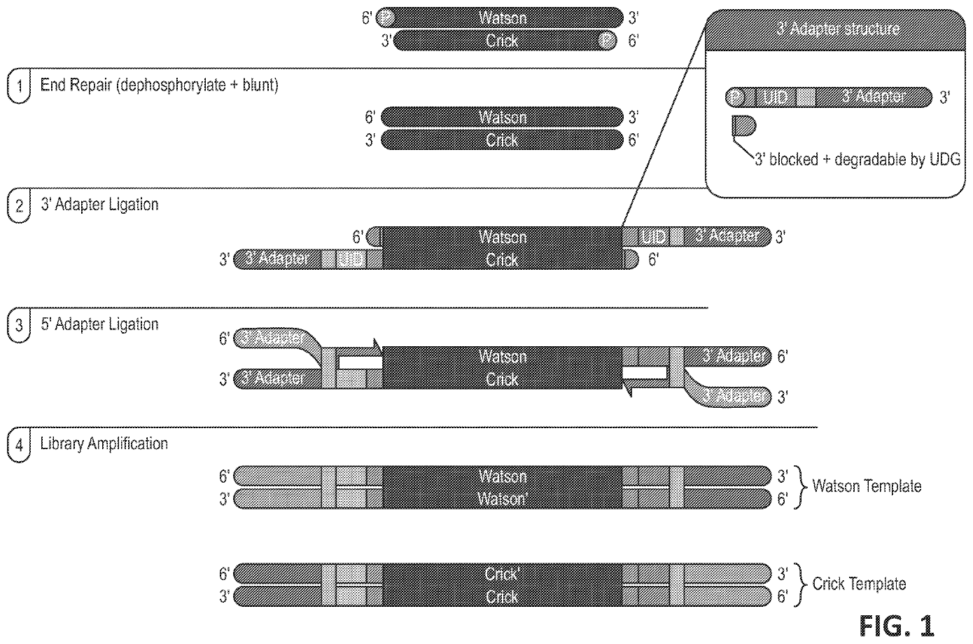

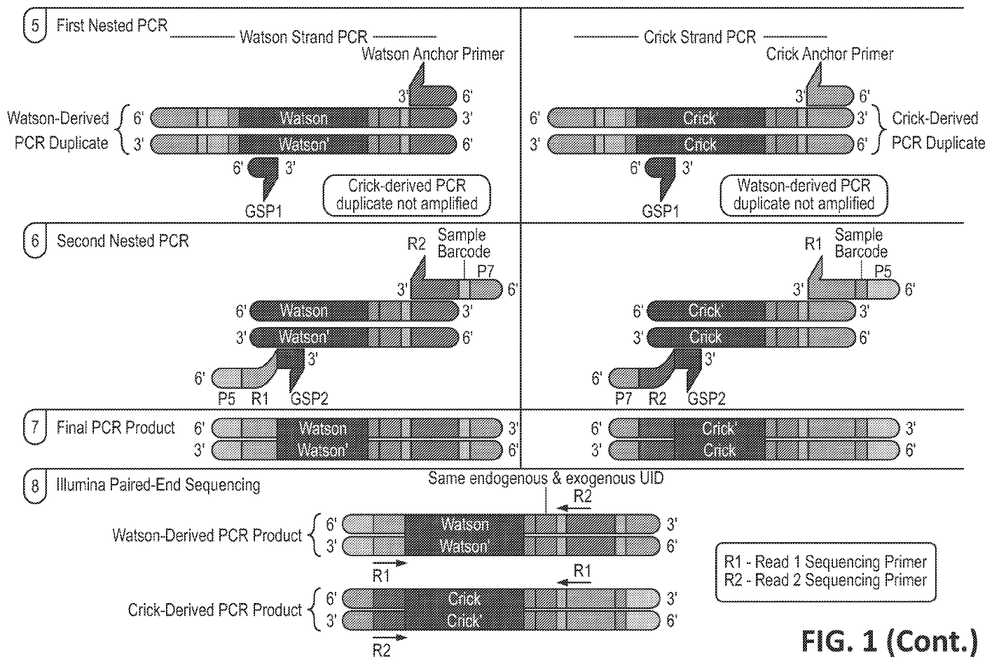

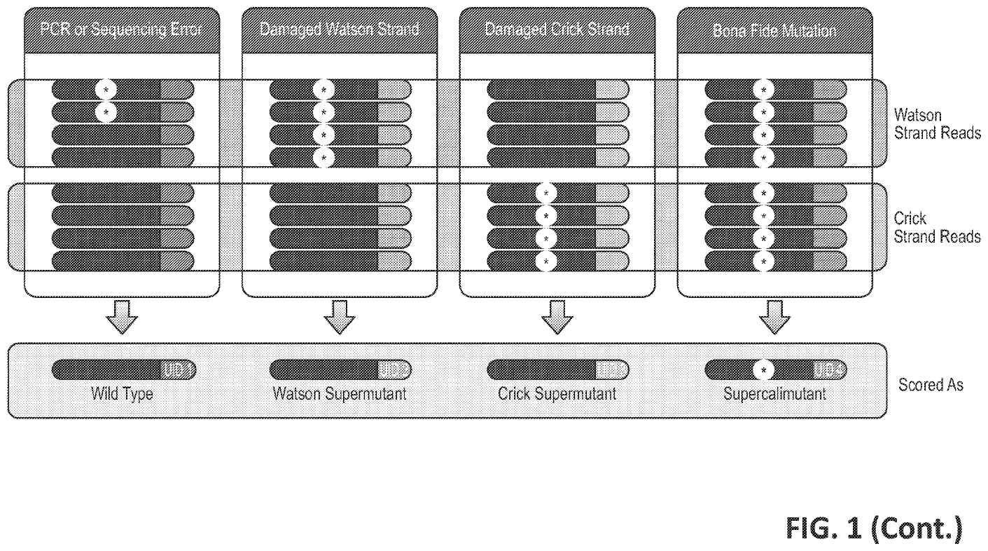

The methods described herein comprise three key steps: i) library construction with in situ generation of double stranded molecular barcodes (FIG. 1a), ii) target enrichment via anchored PCR (FIG. 1b), and iii) in silico reconstruction of template molecules (FIG. 1c). Bona fide sequences present in the original starting templates are identified by requiring the same sequence to be found on both strands of the same initial DNA molecule. This strategy minimizes DNA damage, PCR, and sequencing artifacts and permits the identification of rare sequences with high confidence.

To address inefficiencies and introduced errors typically associated with library construction, a strategy was designed which relies on the sequential ligation of adapter sequences to the 3′ and 5′ DNA fragment ends and the generation of double stranded molecular barcodes in situ (FIG. 1a). The in situ generation of molecular barcodes is the key innovation of the library preparation method. The enzymes used for the in situ generation of double stranded molecular barcodes uniquely barcode each DNA fragment and obviated the need to enzymatically prepare duplex adapters, which has been noted to adversely affect input DNA recovery (FIG. 1a, steps 2 and 3). Following adapter ligation, the fragments are subjected to a limited of number of PCR cycles to create redundant copies of the two original DNA strands (FIG. 1a, step 4).

Another innovation in the protocol disclosed herein is the use of a hemi-nested PCR-based approach for enrichment. Because of the immense combinatorial diversity that is introduced through V(D)J recombination, hemi-nested PCR is ideally suited for enrichment of TCR or BCR sequences. Only a limited set of primers targeting the finite number of J or V segments need to be designed (in contrast to traditional PCR-based methods which employ primers targeting all pairwise V-J segment combinations). Though hemi-nested PCR has previously been used for target enrichment, major changes were required to apply it to duplex sequencing with high efficiency. The previous descriptions of hemi-nested PCR either do not retain the requisite strand information to reconstruct the original duplex molecule or do not recover a high enough fraction of template molecules to permit the detection of variants present at frequencies below 0.1% within limited quantities of DNA. The hemi-nested approach described herein employs two separate PCRs—one for the Watson strand and one for the Crick strand (FIG. 1b). Following sequencing, reads corresponding to each strand of the original DNA duplexes are grouped into Watson and Crick families. Each family member has the identical endogenous barcode representing the sequence at one end of the initial template fragment and the identical exogenous barcode introduced in situ during library construction. Mutations present in a Watson strand family are called “Watson supermutants”. Mutations present in a Crick strand family are called “Crick supermutants”. Those present in both the Watson and Crick families with the same molecular barcode (a “duplex family”) are called “supercalifragilisticexpialidocious mutants”, hereinafter referred to as “supercalimutants” (FIG. 1c).

TCR and BCR sequences can be analyzed using custom or publicly available software packages. In the examples demonstrated here, sequences are grouped for UID error correction using MIGEC, aligned and grouped for clonotypes using MiXCR, and further analyzed using VDJtools.

### Example 2—Hemi-Nested Primers Targeting TCRs

The performance of the methods described herein were evaluated on a sample of DNA derived from human fibroblast cells. For this purpose, hemi-nested primers targeting the 13 TCR J segments were designed. Because fibroblast cells do not undergo V(D)J recombination, this substrate could be used as a suitable template to evaluate the performance of each of the primers designed to enrich the various TCRs.

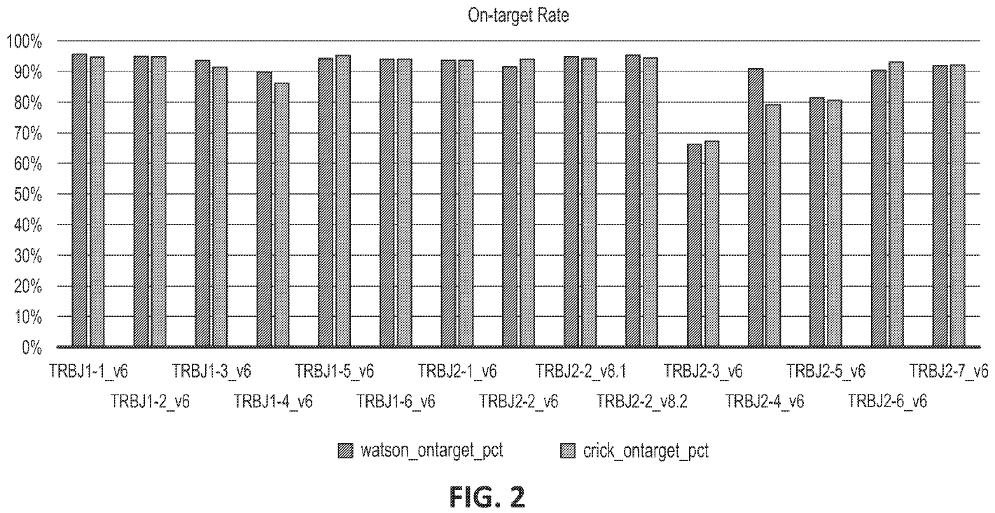

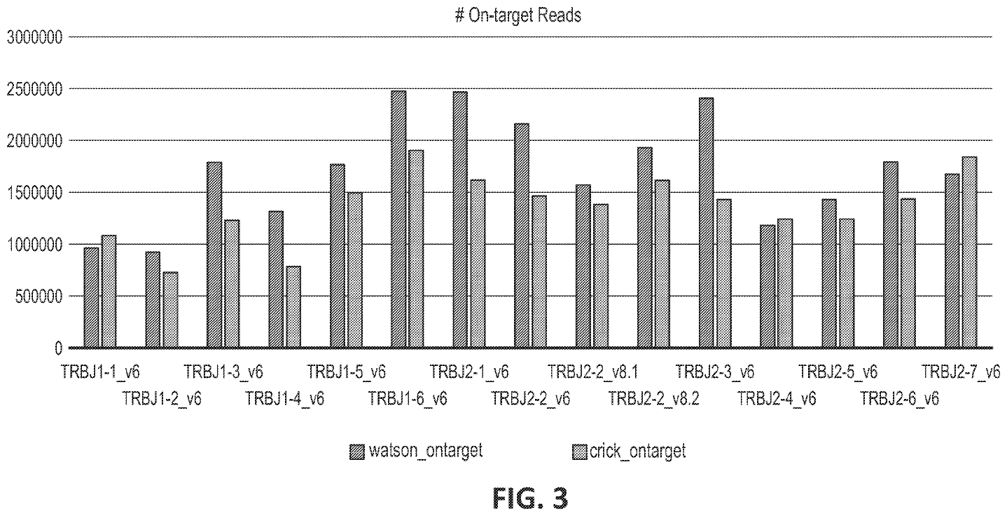

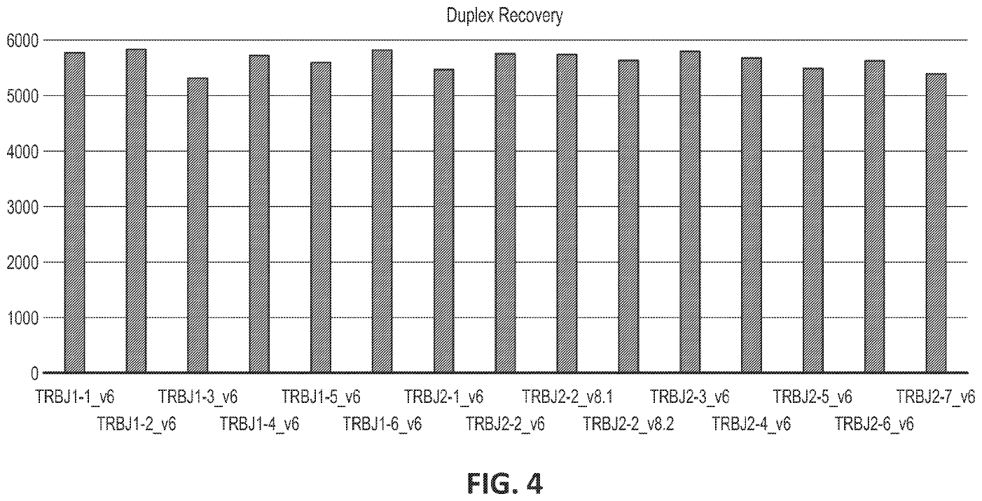

Across the 13 J segment targets, the median fraction of on-target reads (i.e., reads comprised of the intended amplicon) derived from the Watson strand was 94% (range: 66-96%) (FIG. 2). Similarly, the median fraction of on-target reads derived from the Crick strand was 94% (range: 67-85%) (FIG. 2). Each of the targets also exhibited relatively uniform amplification, with coefficients of variation of 29% and 24% for the Watson and Crick-derived reads, respectively (FIG. 3). Finally, the number of duplex UID families (i.e. each UID family represents an original molecule present in the DNA sample) was exceptionally uniform across each of the 13 targets (median: 5,681; range: 5,317-5,835). The coefficient of variation was 2.8% (FIG. 4). Such uniformity is critical for accurate quantification of TCR and BCR sequences.

### Example 3—Synthetic Constructs in Human Fibroblast DNA

Synthetic constructs were designed consisting of, as listed 5′ to 3′, 58 bp of the TRBV2 gene, the Jurkat clone E6-1 CDR3 sequence, a barcode specific for each TRBJ, 50 bp of one of the TRBJ genes (one of TRBJ1-1 to TRBJ2-7), and 50 bp of CMV promoter sequence. These constructs were spiked into normal human fibroblast DNA. Libraries were then prepared for sequencing using each of the TRBJ primer sets in addition to a primer set specific for the CMV sequence. Sequencing reads were grouped by UIDs, aligned sequences, and the number of barcoded molecules identified for each corresponding TRBJ gene synthetic construct were counted.

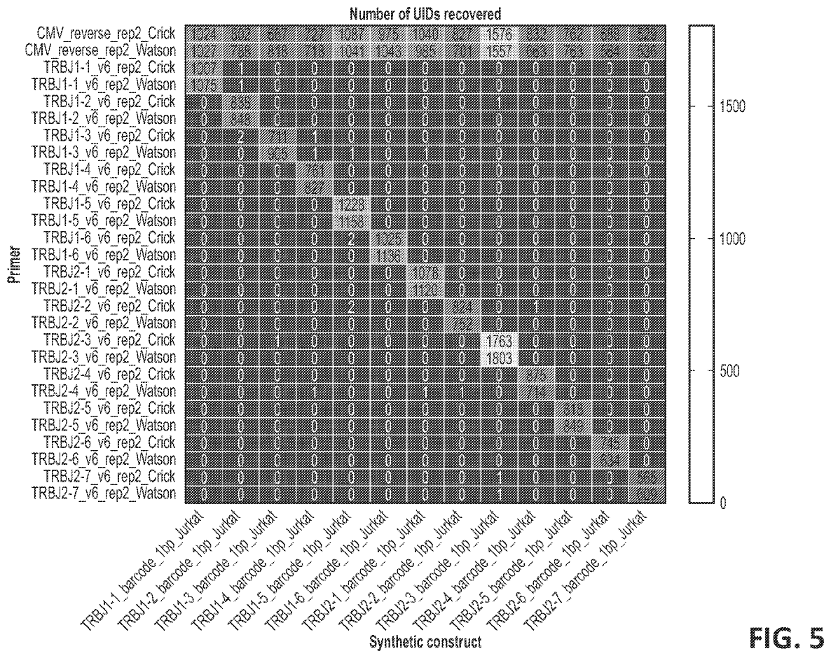

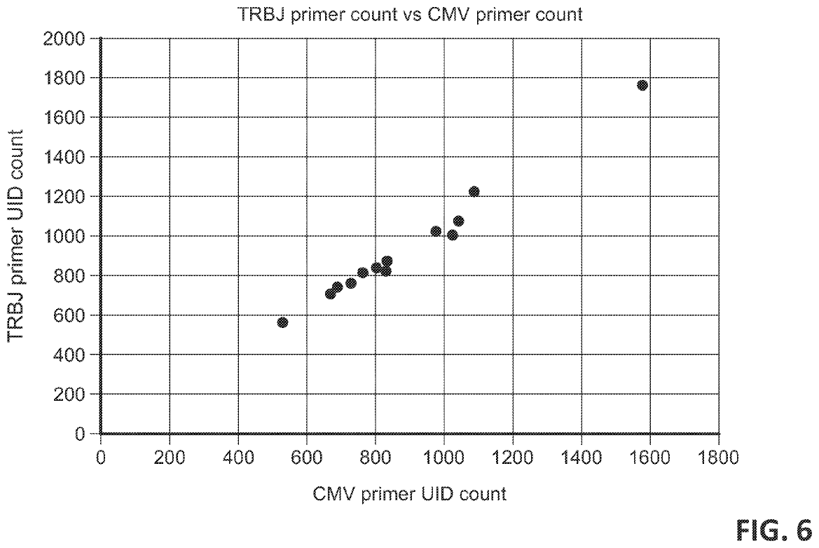

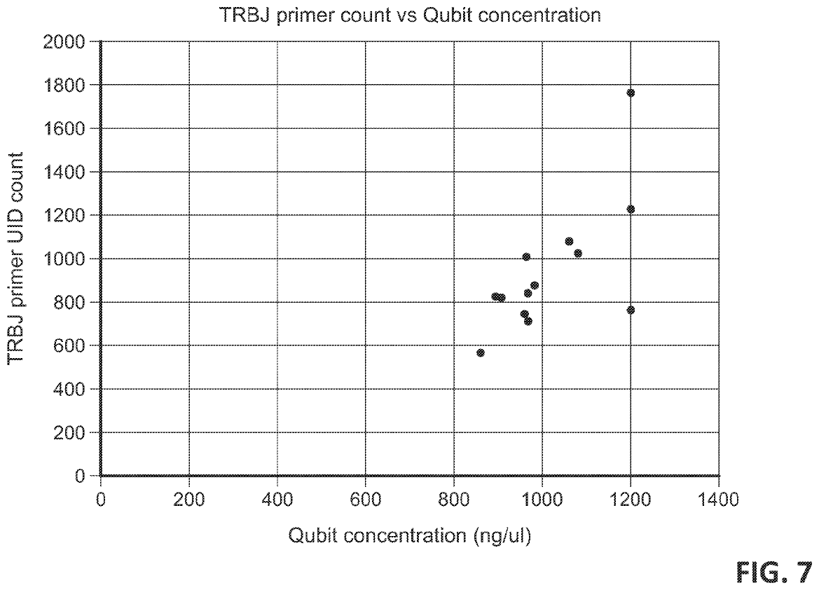

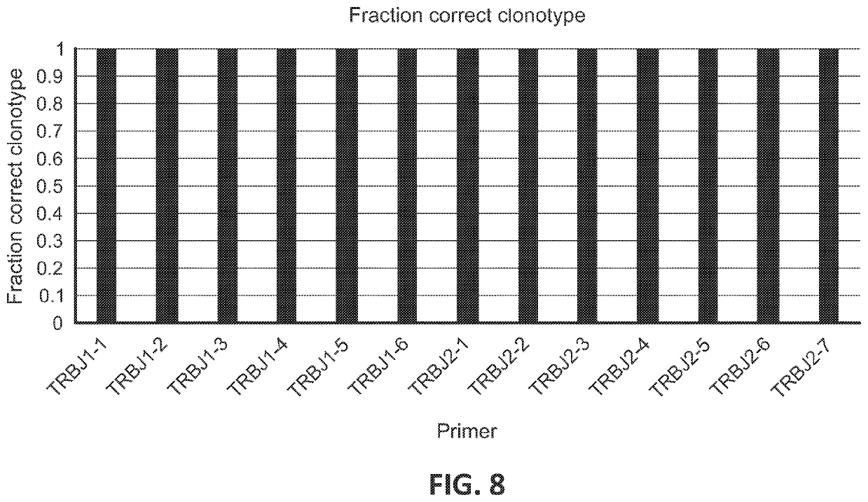

Each TRBJ primer set recovered an approximately equal number of corresponding synthetic construct molecules (median: 833.5; range: 587-1783 for average of Watson and Crick strands) (FIG. 5). Cross-reactive identification of non-corresponding synthetic constructs was minimal (FIG. 5). The number of synthetic construct molecules identified was highly correlated to orthogonal determinations of the synthetic control construct concentrations, as measured by the number of molecules identified using the CMV-specific primer set (FIG. 6) and by concentration in the ThermoFisher Qubit dsDNA HS assay (FIG. 7). The fraction of correct clonotypes identified by each primer set was high (median: 0.999; range 0.998-1.000) (FIG. 8).

### Example 4—Primers to Sequence TCR Receptors

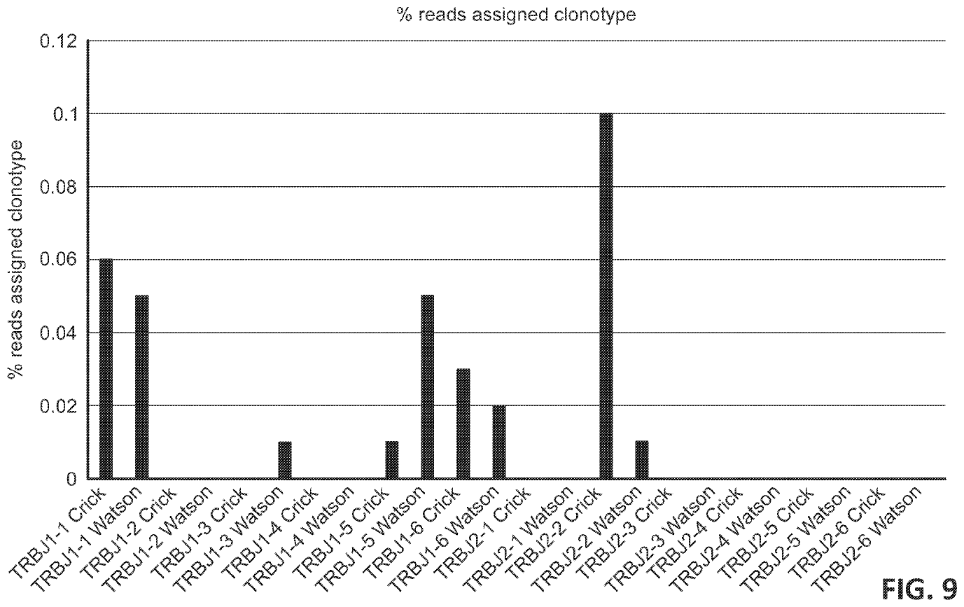

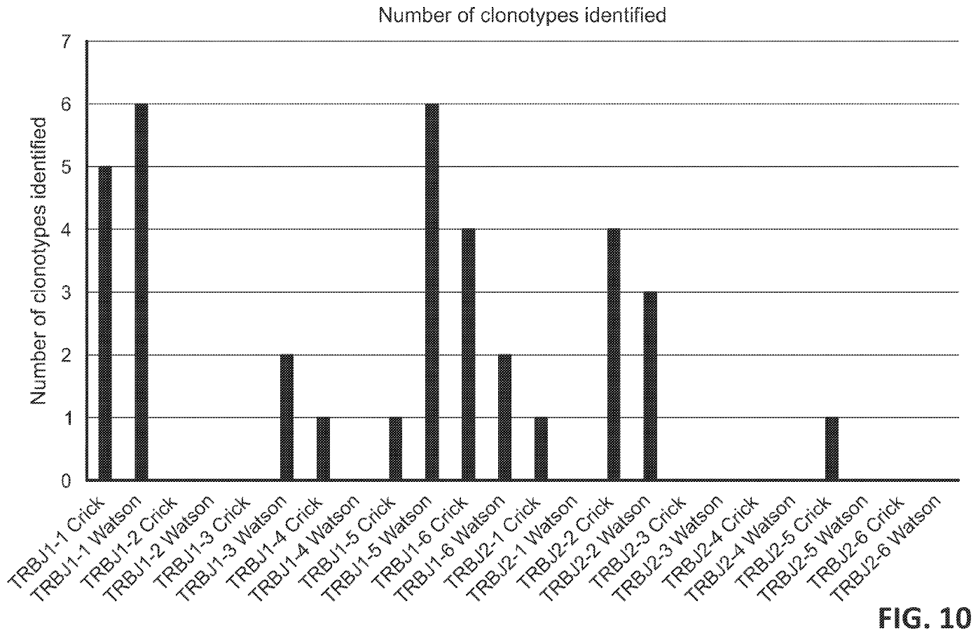

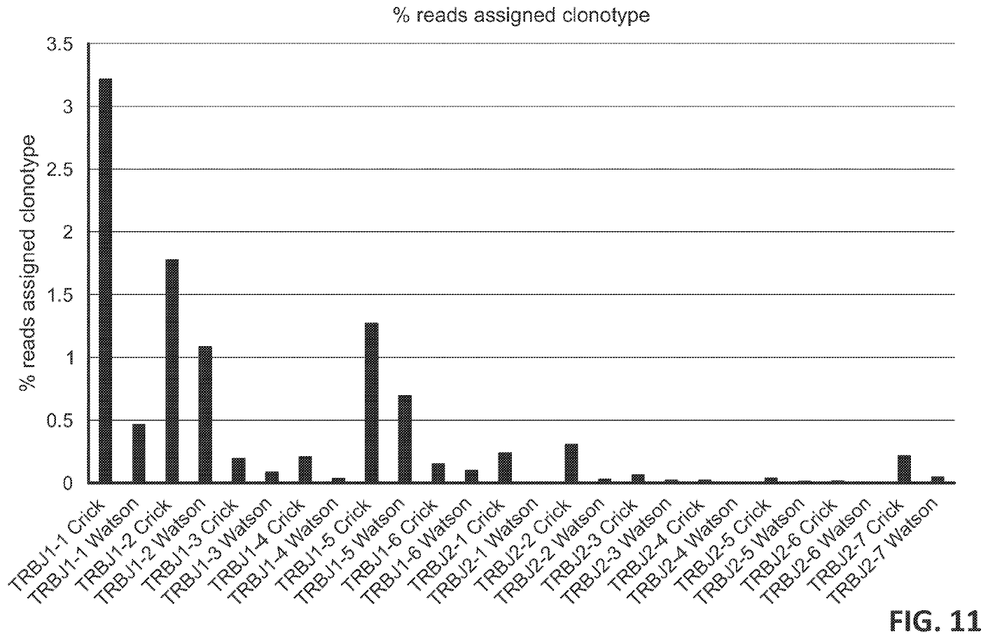

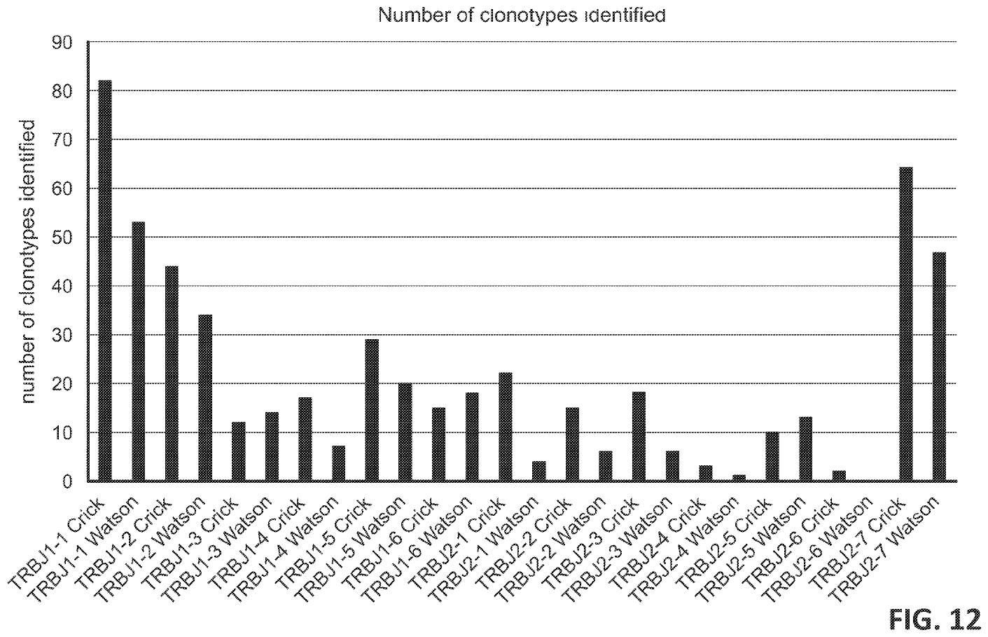

A set of primers (Table 2) was designed to sequence TCR receptors using any one of the methods described herein. The primers were designed to require only 1 round of PCR target enrichment using gene-specific primers. Furthermore, the primers were designed to include and the protocol incorporated RNaseH cleavage with the IDT rhAmpSeq system. The performance of these primers on DNA was evaluated from pooled plasma from normal healthy donors. The percent of sequencing reads assignable to a TCR clonotype was low for all TRBJ segments (range 0-0.06%) (FIG. 9). The number of clonotypes identified was similarly low for all TRBJ segments (range 0-6) (FIG. 10). The performance of the primers and protocols using DNA derived from T cells from a normal healthy donor was also evaluated. The percent of sequencing reads assignable to a TCR clonotype was again low for all TRBJ segments (range 0-3.2%) (FIG. 11). The number of clonotypes identified was also low for all TRBJ segments (range 0-82) (FIG. 12).

TABLE 2

| | Primer Sequence | SEQ ID NO |
|---|---|---|
| TRBJ1-1 | CTACAACTGTGAGTCTGGT | SEQ ID NO: 4 |
| TRBJ1-2 | GCCTTACCTACAACGGTTA | SEQ ID NO: 5 |
| TRBJ1-3 | CCTACAACAGTGAGCCAAC | SEQ ID NO: 6 |
| TRBJ1-4 | AAGACAGAGAGCTGGT | SEQ ID NO: 7 |
| TRBJ1-5 | CCTAGGATGGAGAGTCGAG | SEQ ID NO: 8 |
| TRBJ1-6 | GTCACAGTGAGCCTGGT | SEQ ID NO: 9 |
| TRBJ2-1 | TACCTAGCACGGTGAGC | SEQ ID NO: 10 |
| TRBJ2-2 | CAGTACGGTCAGCCTAGA | SEQ ID NO: 11 |
| TRBJ2-3 | TACCGAGCACTGTCAGC | SEQ ID NO: 12 |
| TRBJ2-4 | CTTACCCAGCACTGAGAG | SEQ ID NO: 13 |
| TRBJ2-5 | CACCGAGCACCAGGAG | SEQ ID NO: 14 |
| TRBJ2-6 | CACCCAGCACGGTCAG | SEQ ID NO: 15 |
| TRBJ2-7 | CTGTGACCGTGAGCCT | SEQ ID NO: 16 |

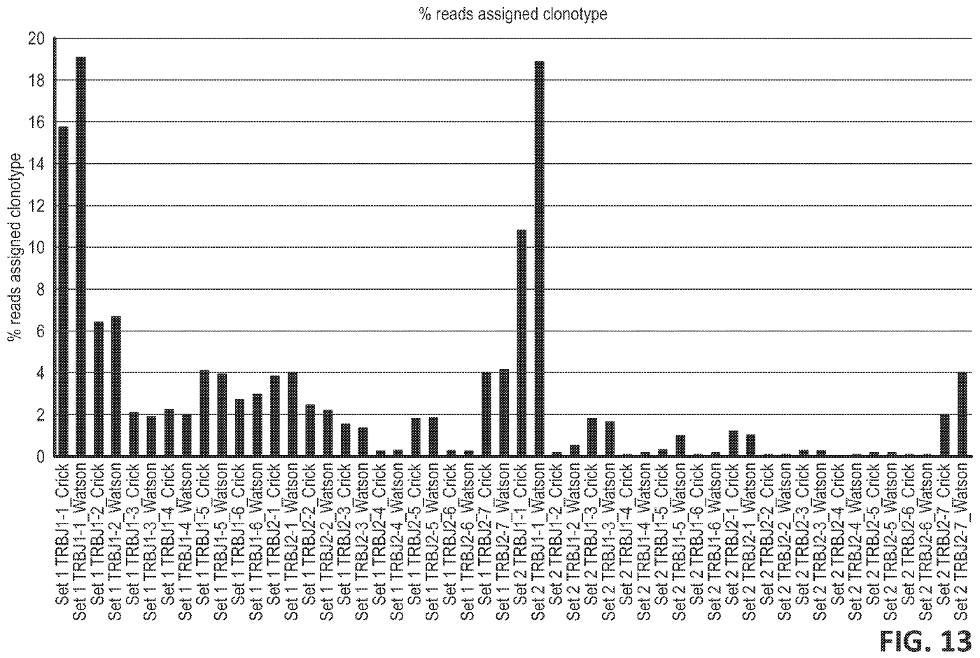

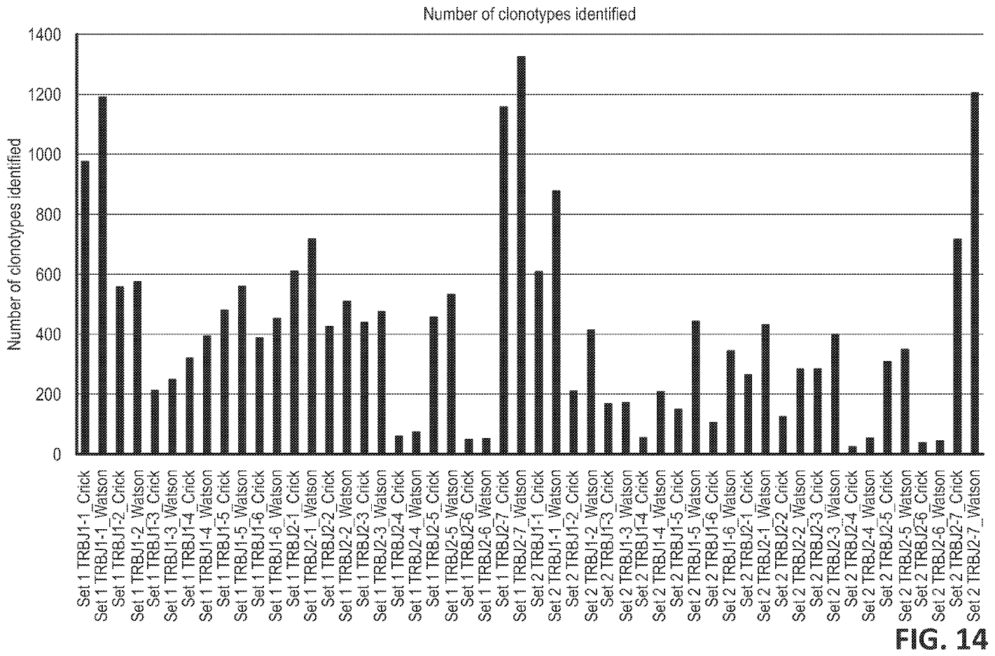

Two different sets of primers were designed to sequence TCR receptors using any one of the methods disclosed herein, entitled here as “Set 1” and “Set 2” (Table 3). The performance of these primer sets on DNA derived from T cells from a normal healthy donor was evaluated. The Set 1 primers had a greater percentage of sequencing reads assignable to TCR clonotypes (FIG. 13) and identified a greater number of clonotypes (FIG. 14) than the Set 2 primers.

TABLE 3

| | GSP1 primer sequence | SEQ ID NO | GSP2 primer sequence | SEQ ID NO |
|---|---|---|---|---|
| Set 1 | cctgaaaaatgtcttacctacaactg | SEQ ID NO: 17 | ctacaactgtgagtctggt | SEQ ID NO: 4 |
| TRBJ1-1 | | | | |
| Set 1 | acccccagccttacct | SEQ ID NO: 18 | gccttacctacaacggtta | SEQ ID NO: 5 |
| TRBJ1-2 | | | | |
| Set 1 | gccttgacttactcacctacaa | SEQ ID NO: 19 | cctacaacagtgagccaac | SEQ ID NO: 6 |
| TRBJ1-3 | | | | |
| Set 1 | gaagtcttttacatacccaagacag | SEQ ID NO: 20 | aagacagagagctgggt | SEQ ID NO: 7 |
| TRBJ1-4 | | | | |
| Set 1 | ccctgattctgcaacttaccta | SEQ ID NO: 21 | cctaggatggagagtcgag | SEQ ID NO: 8 |
| TRBJ1-5 | | | | |
| Set 1 | cccccatacctgtcacag | SEQ ID NO: 22 | gtcacagtgagcctggt | SEQ ID NO: 9 |
| TRBJ1-6 | | | | |
| Set 1 | gagcccccttcttacctag | SEQ ID NO: 23 | tacctagcacggtgagc | SEQ ID NO: 10 |
| TRBJ2-1 | | | | |
| Set 1 | gcctccttacccagtacg | SEQ ID NO: 24 | cagtacggtcagcctaga | SEQ ID NO: 11 |
| TRBJ2-2 | | | | |
| Set 1 | cccgcttaccgagcac | SEQ ID NO: 25 | taccgagcactgtcagc | SEQ ID NO: 12 |
| TRBJ2-3 | | | | |
| Set 1 | ggccccagcttaccca | SEQ ID NO: 26 | cttacccagcactgagag | SEQ ID NO: 13 |
| TRBJ2-4 | | | | |
| Set 1 | gcccgcgctcaccgag | SEQ ID NO: 27 | caccgagcaccaggag | SEQ ID NO: 14 |
| TRBJ2-5 | | | | |
| Set 1 | ccgcgaaaactcaccca | SEQ ID NO: 28 | cacccagcacggtcag | SEQ ID NO: 15 |
| TRBJ2-6 | | | | |
| Set 1 | gcccgaatctcacctgt | SEQ ID NO: 29 | ctgtgaccgtgagcct | SEQ ID NO: 16 |
| TRBJ2-7 | | | | |
| Set 2 | aatgtcttacctacaactgtga | SEQ ID NO: 30 | ctacaactgtgagtctggt | SEQ ID NO: 4 |
| TRBJ1-1 | | | | |
| Set 2 | cccagccttacctacaac | SEQ ID NO: 31 | gccttacctacaacggtta | SEQ ID NO: 5 |
| TRBJ1-2 | | | | |
| Set 2 | ccttgacttactcacctacaacag | SEQ ID NO: 32 | cctacaacagtgagccaac | SEQ ID NO: 6 |
| TRBJ1-3 | | | | |
| Set 2 | tacatacccaagacagagagc | SEQ ID NO: 33 | aagacagagagctgggt | SEQ ID NO: 7 |
| TRBJ1-4 | | | | |
| Set 2 | gcaacttacctaggatggagag | SEQ ID NO: 34 | cctaggatggagagtcgag | SEQ ID NO: 8 |
| TRBJ1-5 | | | | |
| Set 2 | ccatacctgtcacagtgagc | SEQ ID NO: 35 | gtcacagtgagcctggt | SEQ ID NO: 9 |
| TRBJ1-6 | | | | |
| Set 2 | cccttcttacctagcacgg | SEQ ID NO: 36 | tacctagcacggtgagc | SEQ ID NO: 10 |
| TRBJ2-1 | | | | |
| Set 2 | cttacccagtacggtcagc | SEQ ID NO: 37 | cagtacggtcagcctaga | SEQ ID NO: 11 |
| TRBJ2-2 | | | | |
| Set 2 | ccgcttaccgagcactg | SEQ ID NO: 38 | taccgagcactgtcagc | SEQ ID NO: 12 |
| TRBJ2-3 | | | | |
| Set 2 | ccagcttacccagcact | SEQ ID NO: 39 | cttacccagcactgagag | SEQ ID NO: 13 |
| TRBJ2-4 | | | | |
| Set 2 | cgctcaccgagcacca | SEQ ID NO: 40 | caccgagcaccaggag | SEQ ID NO: 14 |
| TRBJ2-5 | | | | |
| Set 2 | gaaaactcacccagcacg | SEQ ID NO: 41 | cacccagcacggtcag | SEQ ID NO: 15 |
| TRBJ2-6 | | | | |
| Set 2 | gaatctcacctgtgaccgt | SEQ ID NO: 42 | ctgtgaccgtgagcct | SEQ ID NO: 16 |
| TRBJ2-7 | | | | |

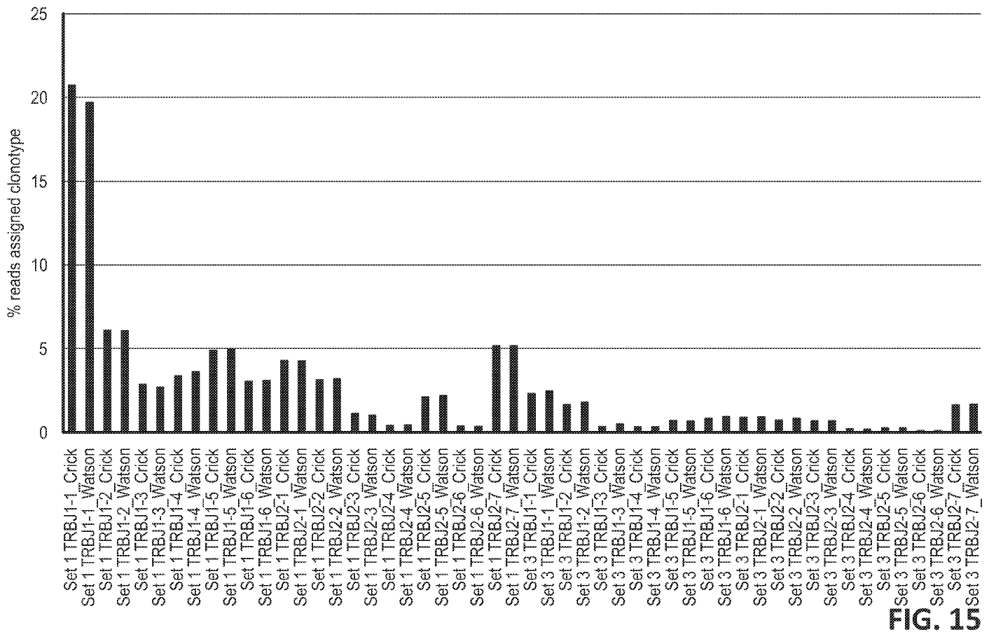

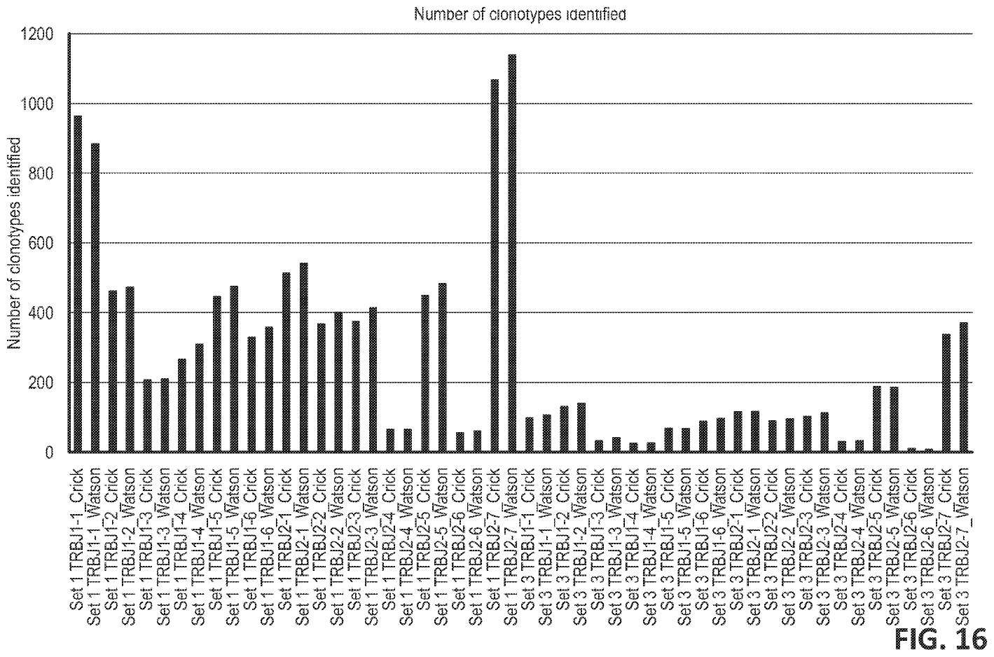

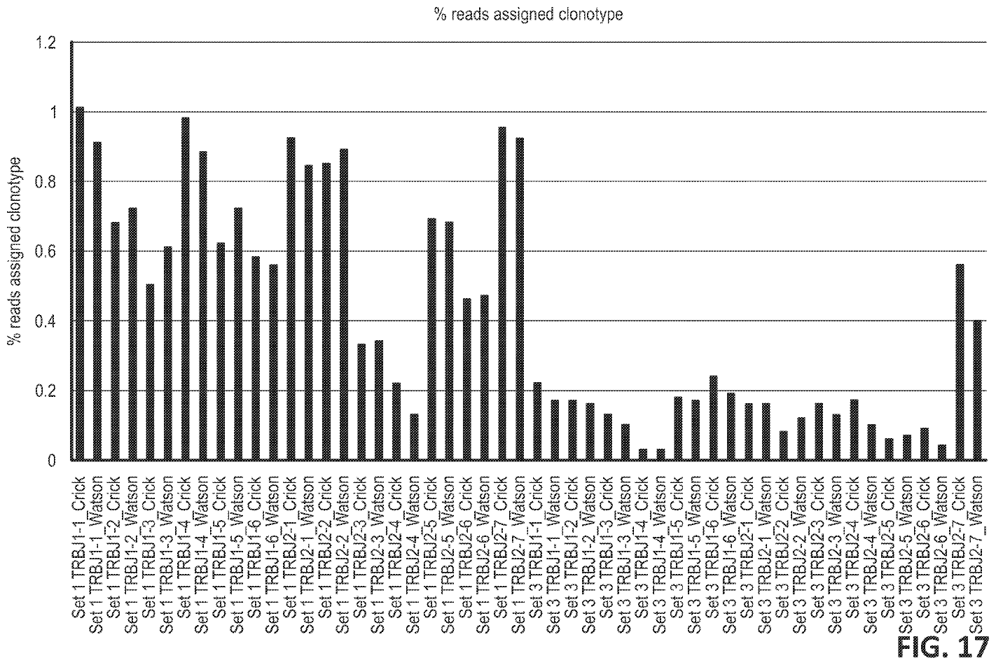

Also, two different sets of primers were designed to sequence TCR receptors using any one of the methods disclosed herein, entitled here as “Set 1” and “Set 3” (Table 4). The performance of these primer sets on DNA derived from T cells from a normal healthy donor were evaluated. The Set 1 primers had a greater percentage of sequencing reads assignable to TCR clonotypes (FIG. 15) and identified a greater number of clonotypes (FIG. 16) than the Set 3 primers. The performance of these primer sets was also evaluated using synthetic control constructs modeling the TCR repertoire. The Set 1 primers had a greater percentage of sequencing reads assignable to TCR clonotypes than the Set 3 primers (FIG. 17).

TABLE 4

| | GSP1 primer sequence | SEQ ID NO | GSP2 primer sequence | SEQ ID NO |
|---|---|---|---|---|
| Set 1 | cctgaaaaatgtcttacctacaactg | SEQ ID NO: 17 | ctacaactgtgagtctggt | SEQ ID NO: 4 |
| TRBJ1-1 | | | | |
| Set 1 | acccccagccttacct | SEQ ID NO: 18 | gccttacctacaacggtta | SEQ ID NO: 5 |
| TRBJ1-2 | | | | |
| Set 1 | gccttgacttactcacctacaa | SEQ ID NO: 19 | cctacaacagtgagccaac | SEQ ID NO: 6 |
| TRBJ1-3 | | | | |
| Set 1 | gaagtcttttacatacccaagacag | SEQ ID NO: 20 | aagacagagagctgggt | SEQ ID NO: 7 |
| TRBJ1-4 | | | | |
| Set 1 | ccctgattctgcaacttaccta | SEQ ID NO: 21 | cctaggatggagagtcgag | SEQ ID NO: 8 |
| TRBJ1-5 | | | | |
| Set 1 | cccccatacctgtcacag | SEQ ID NO: 22 | gtcacagtgagcctggt | SEQ ID NO: 9 |
| TRBJ1-6 | | | | |
| Set 1 | gagcccccttcttacctag | SEQ ID NO: 23 | tacctagcacggtgagc | SEQ ID NO: 10 |
| TRBJ2-1 | | | | |
| Set 1 | gcctccttacccagtacg | SEQ ID NO: 24 | cagtacggtcagcctaga | SEQ ID NO: 11 |
| TRBJ2-2 | | | | |
| Set 1 | cccgcttaccgagcac | SEQ ID NO: 25 | taccgagcactgtcagc | SEQ ID NO: 12 |
| TRBJ2-3 | | | | |
| Set 1 | ggccccagcttaccca | SEQ ID NO: 26 | cttacccagcactgagag | SEQ ID NO: 13 |
| TRBJ2-4 | | | | |
| Set 1 | gcccgcgctcaccgag | SEQ ID NO: 27 | caccgagcaccaggag | SEQ ID NO: 14 |
| TRBJ2-5 | | | | |
| Set 1 | ccgcgaaaactcaccca | SEQ ID NO: 28 | cacccagcacggtcag | SEQ ID NO: 15 |
| TRBJ2-6 | | | | |
| Set 1 | gcccgaatctcacctgt | SEQ ID NO: 29 | ctgtgaccgtgagcct | SEQ ID NO: 16 |
| TRBJ2-7 | | | | |
| Set 3 | gcaaaagaacctgaaaaatgtctta | SEQ ID NO: 43 | ctgaaaaatgtcttacctacaactg | SEQ ID NO: 56 |
| TRBJ1-1 | | | | |
| Set 3 | ccctcctagagaccccc | SEQ ID NO: 44 | gacccccagccttacct | SEQ ID NO: 57 |
| TRBJ1-2 | | | | |
| Set 3 | ctgtccagccttgacttactc | SEQ ID NO: 45 | ccttgacttactcacctacaaca | SEQ ID NO: 58 |
| TRBJ1-3 | | | | |
| Set 3 | ctatcccgaaagaagtcttttacata | SEQ ID NO: 46 | gaagtcttttacatacccaagacag | SEQ ID NO: 59 |
| TRBJ1-4 | | | | |
| Set 3 | ccaccctgattctgcaactt | SEQ ID NO: 47 | tgattctgcaacttacctaggatg | SEQ ID NO: 60 |
| TRBJ1-5 | | | | |
| Set 3 | aagagtggagcccccata | SEQ ID NO: 48 | cccccatacctgtcacag | SEQ ID NO: 61 |
| TRBJ1-6 | | | | |
| Set 3 | ccacctggagccccct | SEQ ID NO: 49 | gagcccccttcttacctag | SEQ ID NO: 62 |
| TRBJ2-1 | | | | |
| Set 3 | ccccaaccgcctcctta | SEQ ID NO: 50 | gcctccttacccagtacgg | SEQ ID NO: 63 |
| TRBJ2-2 | | | | |
| Set 3 | ttcagcgggagccccc | SEQ ID NO: 51 | gcccccgcttaccgag | SEQ ID NO: 64 |
| TRBJ2-3 | | | | |
| Set 3 | cggcggccccagctta | SEQ ID NO: 52 | cccagcttacccagcac | SEQ ID NO: 65 |
| TRBJ2-4 | | | | |
| Set 3 | cagcagcccgcgctca | SEQ ID NO: 53 | cgcgctcaccgagcac | SEQ ID NO: 66 |
| TRBJ2-5 | | | | |
| Set 3 | gggtggtcccgcgaaa | SEQ ID NO: 54 | ccgcgaaaactcaccca | SEQ ID NO: 67 |
| TRBJ2-6 | | | | |
| Set 3 | ggagacgcccgaatctca | SEQ ID NO: 55 | cgaatctcacctgtgaccg | SEQ ID NO: 68 |
| TRBJ2-7 | | | | |

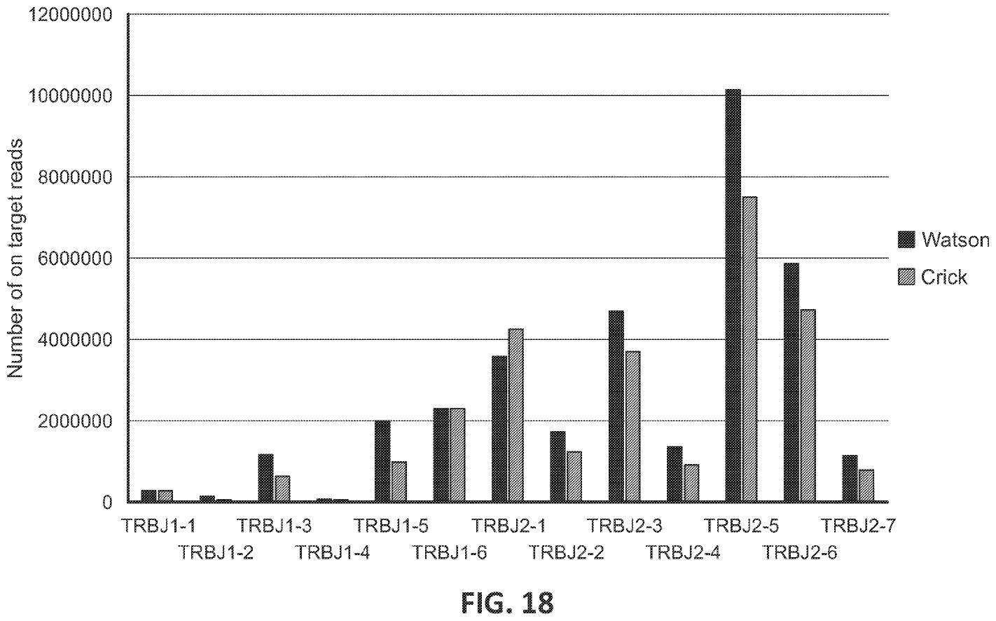

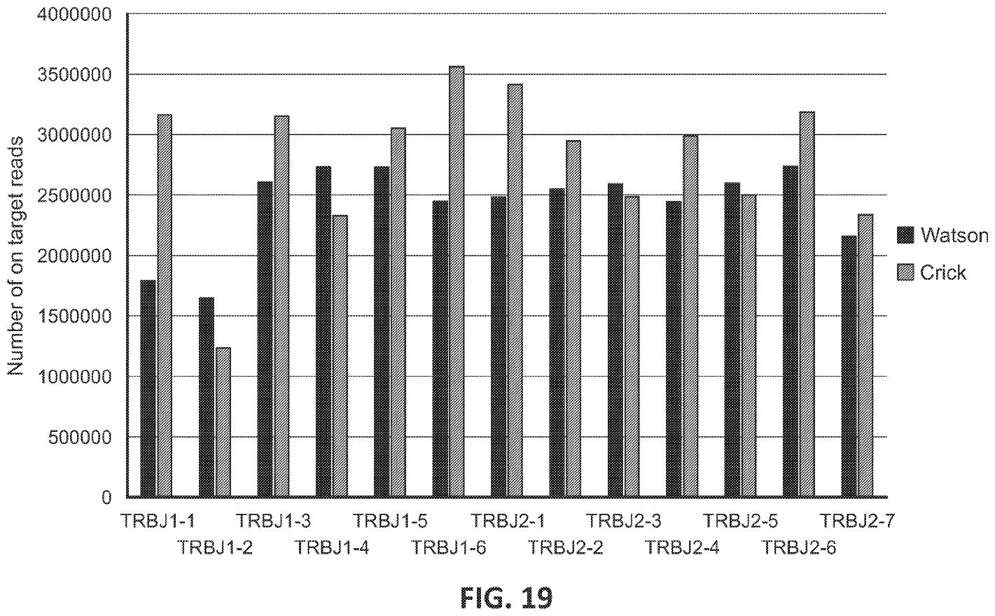

Multiplex primer sets were also created with the TRBJ primers pooled in equimolar ratios or pooled in a ratio designed to achieved balanced reads for each TRBJ segment, entitled here as “Multiplex Pool 1” and “Multiplex Pool 2” respectively (Table 5). The performance of these primer sets on DNA derived from fibroblasts from a normal healthy donor was evaluated. The coefficient of variation for the number of on-target reads for each TRBJ segment for Multiplex Pool 1 was 103.5% (FIG. 18). Multiplex Pool 2 exhibited more balanced recovery of each TRBJ segment with a coefficient of variation for the number of on-target reads for TRBJ segment of 17.5% (FIG. 19).

TABLE 5

| Primer | Relative ratio |
|---|---|
| Multiplex Pool 1 TRBJ1-1 | 1.0 |
| Multiplex Pool 1 TRBJ1-2 | 1.0 |
| Multiplex Pool 1 TRBJ1-3 | 1.0 |
| Multiplex Pool 1 TRBJ1-4 | 1.0 |
| Multiplex Pool 1 TRBJ1-5 | 1.0 |
| Multiplex Pool 1 TRBJ1-6 | 1.0 |
| Multiplex Pool 1 TRBJ2-1 | 1.0 |
| Multiplex Pool 1 TRBJ2-2 | 1.0 |
| Multiplex Pool 1 TRBJ2-3 | 1.0 |
| Multiplex Pool 1 TRBJ2-4 | 1.0 |
| Multiplex Pool 1 TRBJ2-5 | 1.0 |
| Multiplex Pool 1 TRBJ2-6 | 1.0 |
| Multiplex Pool 1 TRBJ2-7 | 1.0 |
| Multiplex Pool 2 TRBJ1-1 | 7.3 |
| Multiplex Pool 2 TRBJ1-2 | 11.9 |
| Multiplex Pool 2 TRBJ1-3 | 3.6 |
| Multiplex Pool 2 TRBJ1-4 | 11.9 |
| Multiplex Pool 2 TRBJ1-5 | 2.8 |
| Multiplex Pool 2 TRBJ1-6 | 2.3 |
| Multiplex Pool 2 TRBJ2-1 | 2.2 |
| Multiplex Pool 2 TRBJ2-2 | 3.9 |
| Multiplex Pool 2 TRBJ2-3 | 2.9 |
| Multiplex Pool 2 TRBJ2-4 | 4.9 |
| Multiplex Pool 2 TRBJ2-5 | 1.0 |
| Multiplex Pool 2 TRBJ2-6 | 1.3 |
| Multiplex Pool 2 TRBJ2-7 | 5.7 |

### Example 5-Multiplex Primer Sets with TRBJ Primers

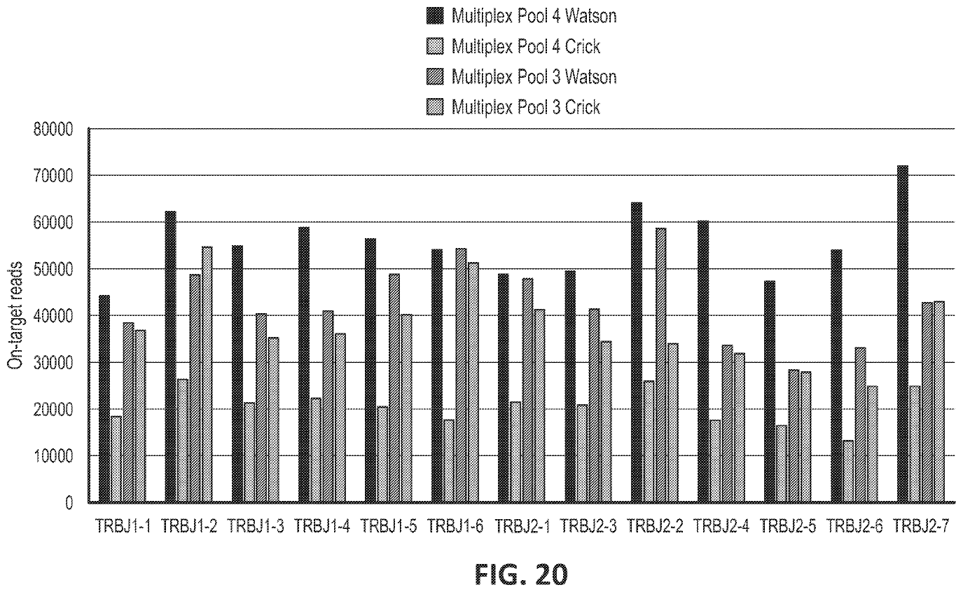

Multiplex primer sets were created with the TRBJ primers pooled in ratios designed to achieve balanced reads for each TRBJ segment. The ratios of primers in each mix were adjusted based on the ratio of reads with the mix described above. The ratios of primers were adjusted separately for Watson and Crick GSP reactions. The performance of these primer sets were evaluated on DNA derived from fibroblasts from a normal healthy donor. The penultimate set of primer pools is entitled here “Multiplex Pool 3” and the final set of primer pools is entitled here “Multiplex Pool 4” (Table 6). The coefficient of variation for the number of on-target reads for each TRBJ segment for Multiplex Pool 3 was 19.4% for Watson and 21.4% for Crick (FIG. 20). Multiplex Pool 4 exhibited more balanced recovery of each TRBJ segment with a coefficient of variation for the number of on-target reads for each TRBJ segment of 13.2% for Watson and 18.1% for Crick (FIG. 20).

TABLE 6

| Primer | Relative Ratio |
|---|---|
| Multiplex Pool 3 Watson TRBJ1-1 | 8.02 |
| Multiplex Pool 3 Watson TRBJ1-2 | 6.24 |
| Multiplex Pool 3 Watson TRBJ1-3 | 5.17 |
| Multiplex Pool 3 Watson TRBJ1-4 | 5.89 |
| Multiplex Pool 3 Watson TRBJ1-5 | 5.48 |
| Multiplex Pool 3 Watson TRBJ1-6 | 3.50 |
| Multiplex Pool 3 Watson TRBJ2-1 | 4.24 |
| Multiplex Pool 3 Watson TRBJ2-2 | 6.41 |
| Multiplex Pool 3 Watson TRBJ2-3 | 3.06 |
| Multiplex Pool 3 Watson TRBJ2-4 | 17.95 |
| Multiplex Pool 3 Watson TRBJ2-5 | 13.49 |
| Multiplex Pool 3 Watson TRBJ2-6 | 12.44 |
| Multiplex Pool 3 Watson TRBJ2-7 | 8.11 |
| Multiplex Pool 3 Crick TRBJ1-1 | 5.41 |
| Multiplex Pool 3 Crick TRBJ1-2 | 4.49 |
| Multiplex Pool 3 Crick TRBJ1-3 | 4.04 |
| Multiplex Pool 3 Crick TRBJ1-4 | 4.22 |
| Multiplex Pool 3 Crick TRBJ1-5 | 11.57 |
| Multiplex Pool 3 Crick TRBJ1-6 | 2.00 |
| Multiplex Pool 3 Crick TRBJ2-1 | 3.22 |
| Multiplex Pool 3 Crick TRBJ2-2 | 6.25 |
| Multiplex Pool 3 Crick TRBJ2-3 | 3.20 |
| Multiplex Pool 3 Crick TRBJ2-4 | 16.11 |
| Multiplex Pool 3 Crick TRBJ2-5 | 14.12 |
| Multiplex Pool 3 Crick TRBJ2-6 | 15.29 |
| Multiplex Pool 3 Crick TRBJ2-7 | 10.09 |
| Multiplex Pool 4 Watson TRBJ1-1 | 6.94 |
| Multiplex Pool 4 Watson TRBJ1-2 | 5.2 |
| Multiplex Pool 4 Watson TRBJ1-3 | 4.63 |
| Multiplex Pool 4 Watson TRBJ1-4 | 6.09 |
| Multiplex Pool 4 Watson TRBJ1-5 | 4.30 |
| Multiplex Pool 4 Watson TRBJ1-6 | 2.50 |
| Multiplex Pool 4 Watson TRBJ2-1 | 3.19 |
| Multiplex Pool 4 Watson TRBJ2-2 | 5.61 |
| Multiplex Pool 4 Watson TRBJ2-3 | 2.32 |
| Multiplex Pool 4 Watson TRBJ2-4 | 25.65 |
| Multiplex Pool 4 Watson TRBJ2-5 | 11.83 |
| Multiplex Pool 4 Watson TRBJ2-6 | 12.04 |
| Multiplex Pool 4 Watson TRBJ2-7 | 9.70 |
| Multiplex Pool 4 Crick TRBJ1-1 | 4.86 |
| Multiplex Pool 4 Crick TRBJ1-2 | 2.84 |
| Multiplex Pool 4 Crick TRBJ1-3 | 4.61 |
| Multiplex Pool 4 Crick TRBJ1-4 | 4.15 |
| Multiplex Pool 4 Crick TRBJ1-5 | 8.82 |
| Multiplex Pool 4 Crick TRBJ1-6 | 1.35 |
| Multiplex Pool 4 Crick TRBJ2-1 | 3.11 |
| Multiplex Pool 4 Crick TRBJ2-2 | 7.89 |
| Multiplex Pool 4 Crick TRBJ2-3 | 4.12 |
| Multiplex Pool 4 Crick TRBJ2-4 | 16.30 |
| Multiplex Pool 4 Crick TRBJ2-5 | 14.47 |
| Multiplex Pool 4 Crick TRBJ2-6 | 17.61 |
| Multiplex Pool 4 Crick TRBJ2-7 | 9.88 |

### Example 6—Evaluation of Performance by Determining Yield

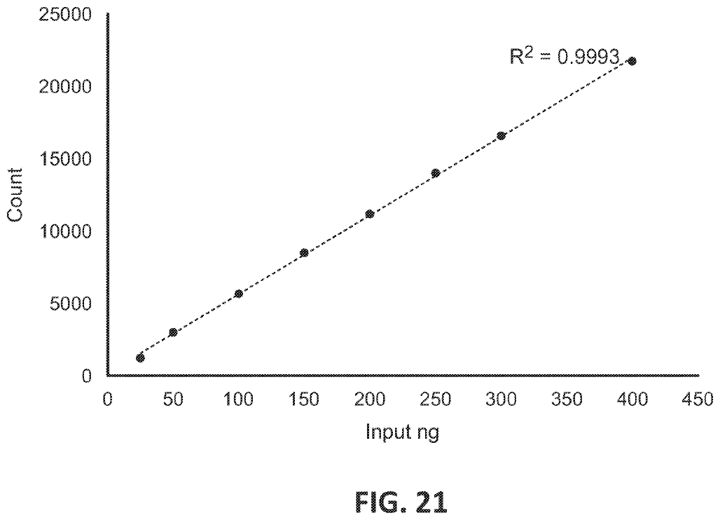

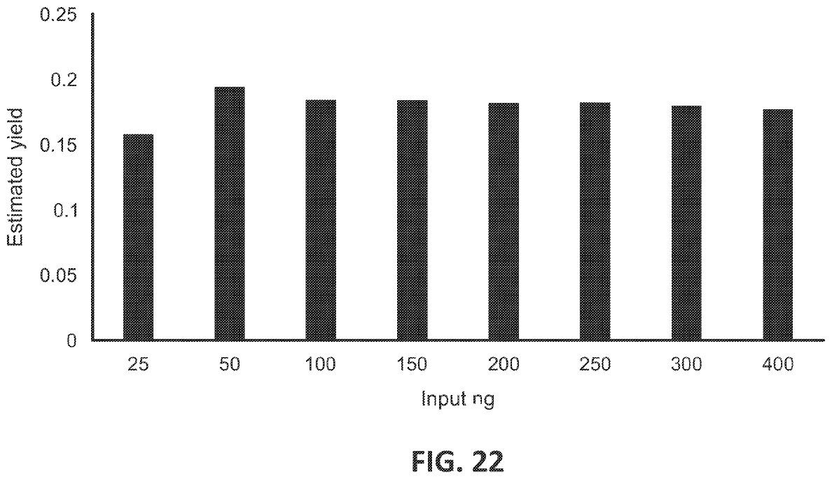

The performance of the methods described herein were evaluated by determining the yield with varying amounts of input DNA derived from healthy donor T cells. The number of TCRs recovered, averaged from donors and replicates, was linear across input amounts from 25 ng to 400 ng (FIG. 21). The yield, averaged for donors and replicates, was also consistent across input amounts from 25 ng to 400 ng (FIG. 22).

### Example 7—Epstein-Barr Virus (EBV) Specific T Cells

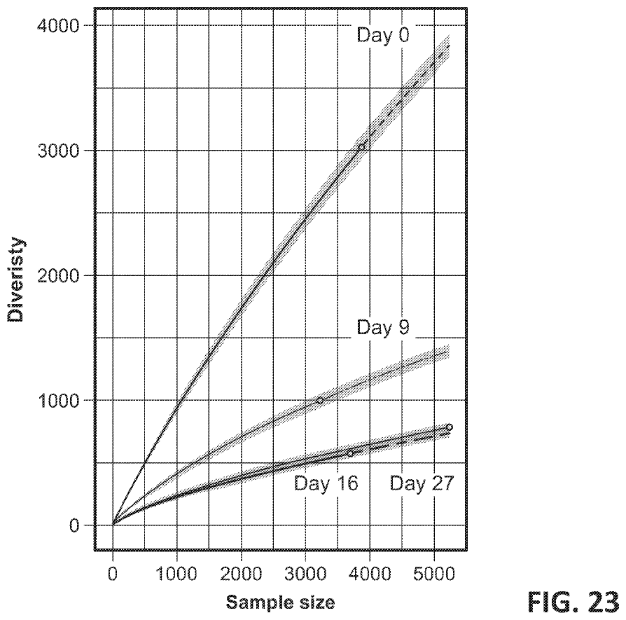

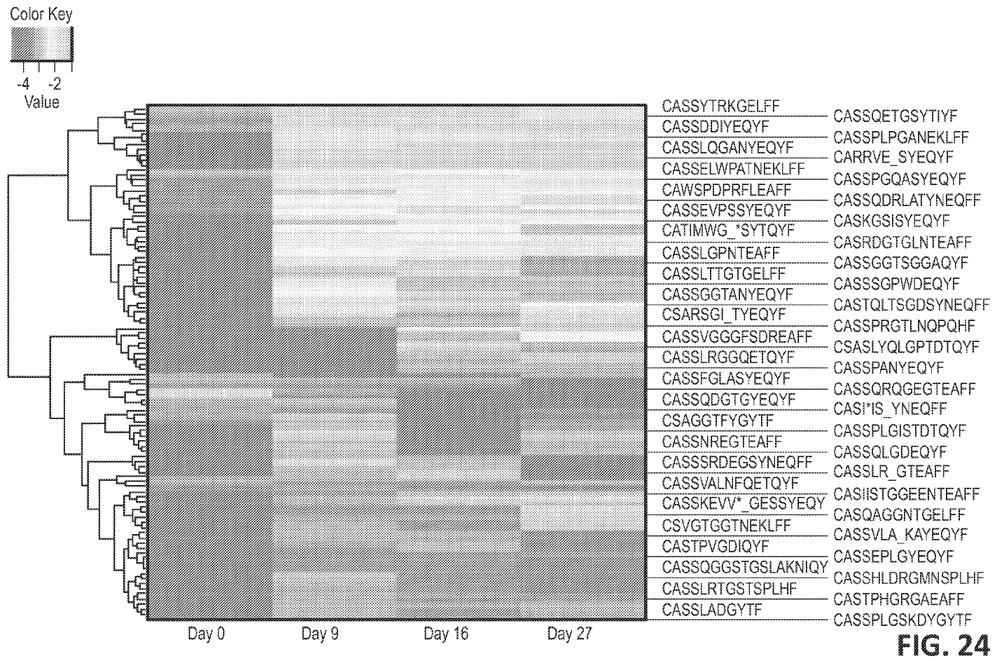

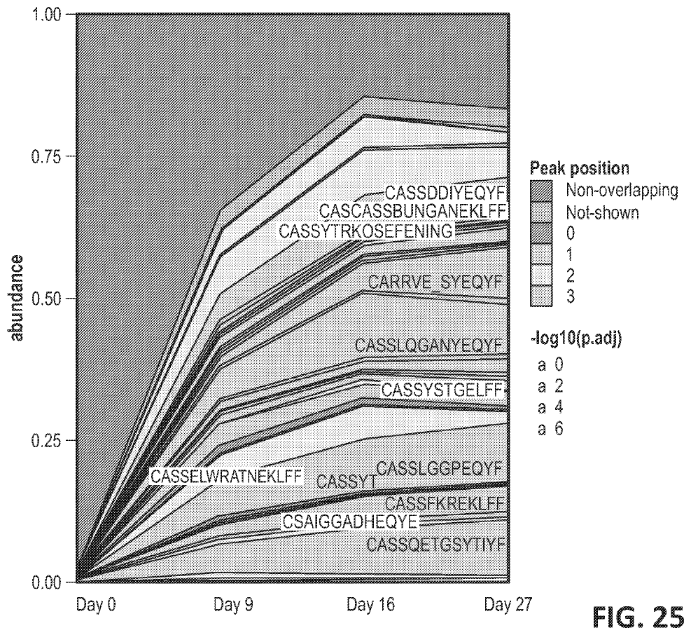

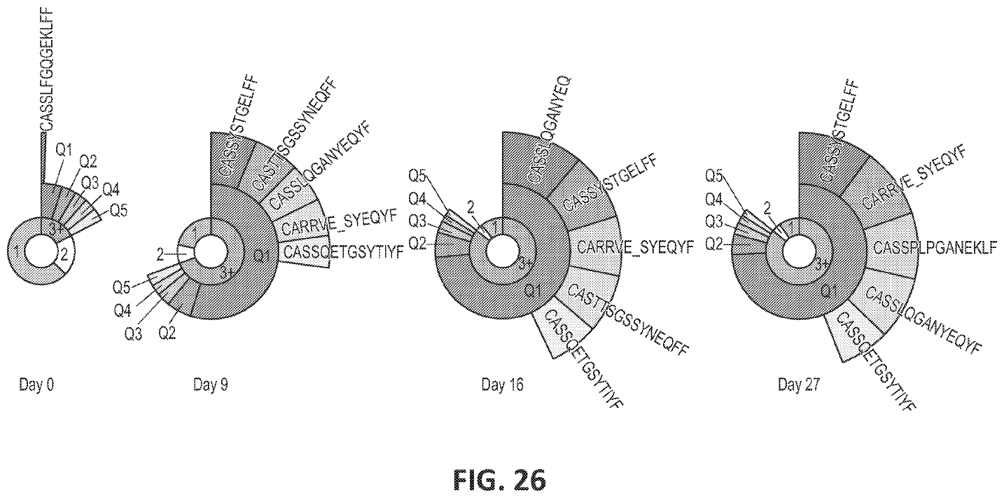

Epstein-Barr Virus (EBV) specific T cells were expanded using EBV peptides. T cells were collected on day 0, 9, 16, and 27 of expansion. The TCR repertoires of the cells were evaluated using the methods described herein. Analysis was correctly demonstrated a reduction in clonal diversity over the course of the expansion (FIG. 23). The methods also identified the outgrowth of specific clones over the course of expansion (FIG. 24, FIG. 25, FIG. 26).

### Example 8—Identification of TCR Sequences from Extracted DNA

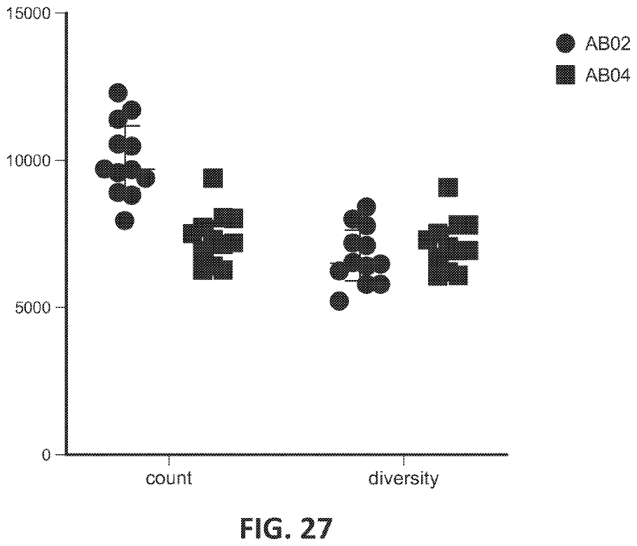

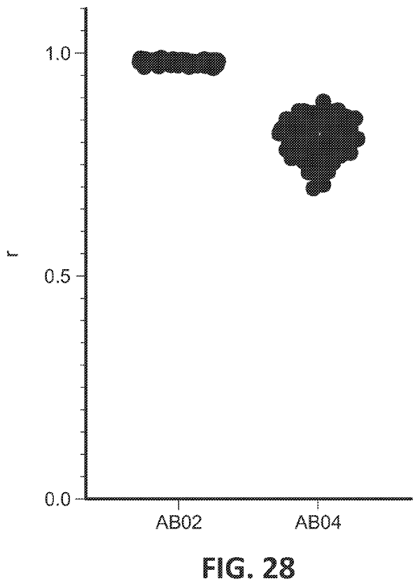

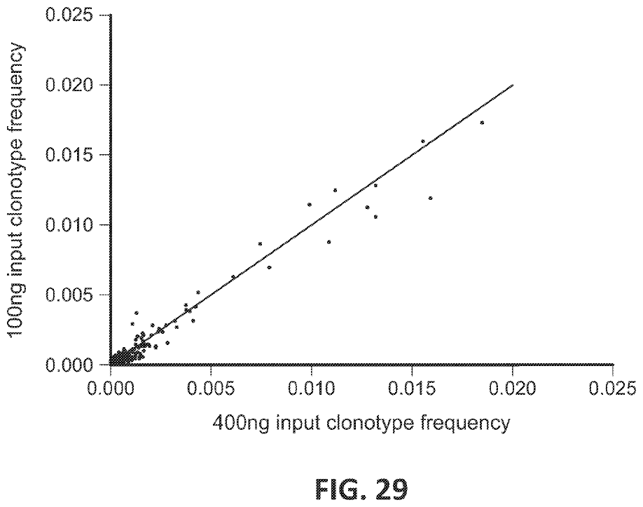

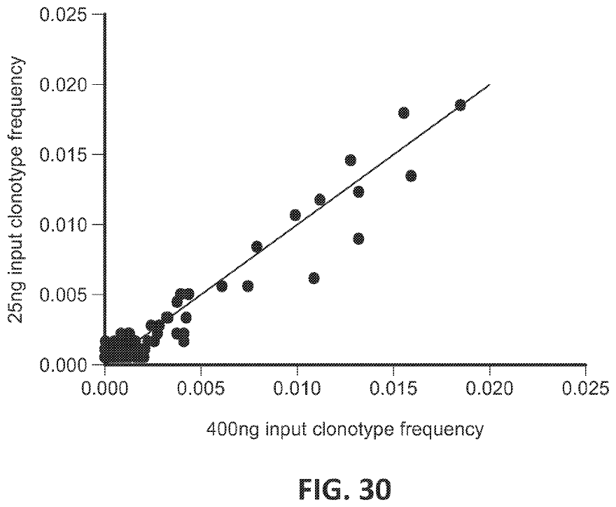

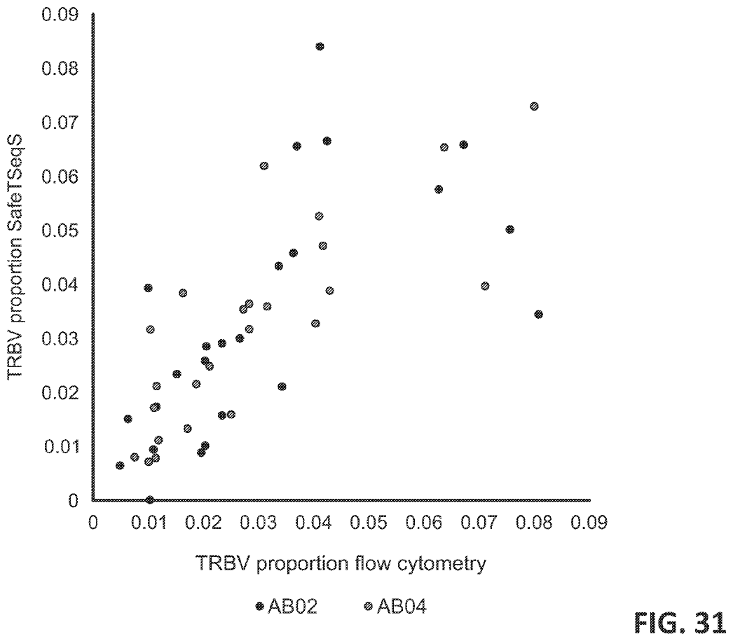

DNA was extracted from T cells derived from two healthy donors, designated “AB02” and “AB04.” The TCR repertoires were analyzed using the methods described herein from multiple replicates and DNA input amounts from these samples. The number of TCRs recovered and the diversity of TCRs recovered was high for both donors across all replicates (FIG. 27). The pairwise distance correlation between replicates from each donor was consistently high (FIG. 28). The clonotype frequencies in samples from DNA input amounts of 400 ng, 100 ng, and 25 ng were well correlated for representative donor AB02 (FIG. 29, FIG. 30). In addition, the TCR V segment gene usage was analyzed in the T cell populations by flow cytometry using the Beckman Coulter IOTest Beta Mark TCR VB Repertoire Kit. The proportion of V gene segment usage was well correlated with the proportion of V gene segment usage measured by flow cytometry (FIG. 31).

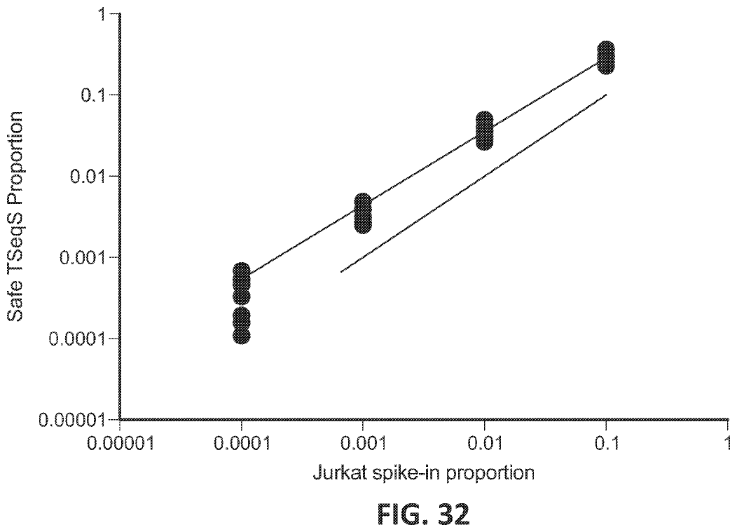

DNA was isolated from the Jurkat clonal T cell line. This DNA was spiked into DNA derived from healthy donor T cells in varying amounts. The TCR repertoires were analyzed using methods described herein. The proportion of TCR reads corresponding to the Jurkat clone TCR was well correlated with the input DNA proportion (FIG. 32).

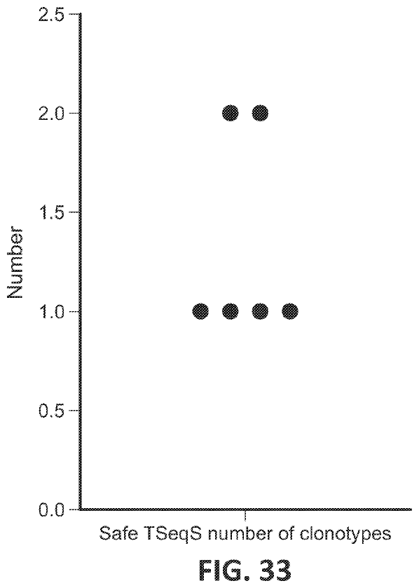

The methods described herein were used to analyze DNA derived from the Jurkat clonal T cell line. The methods correctly identified exactly 1 TCR clone in the Jurkat sample for 4 replicates with 2 clones called in 2 replicates (FIG. 33).

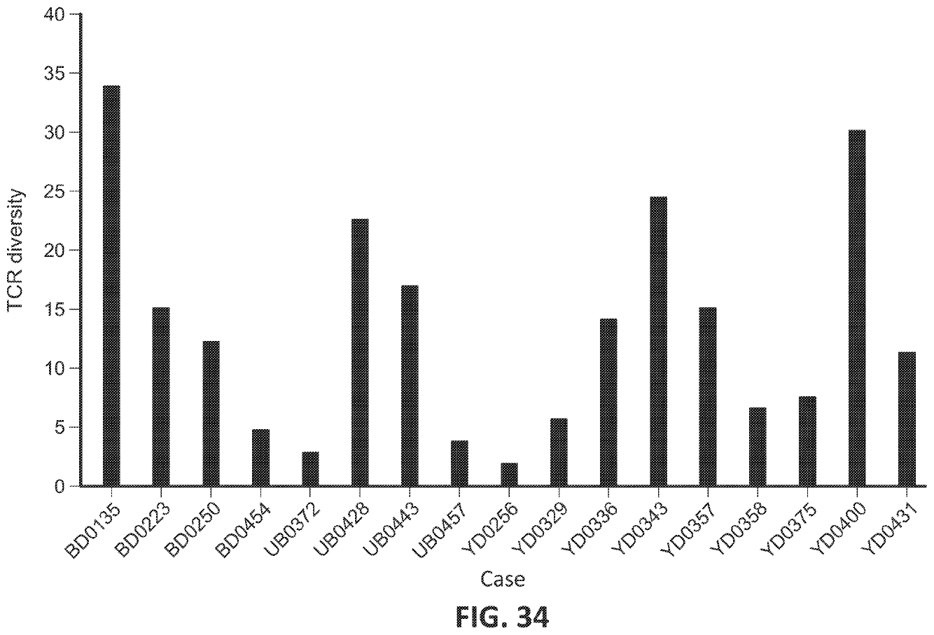

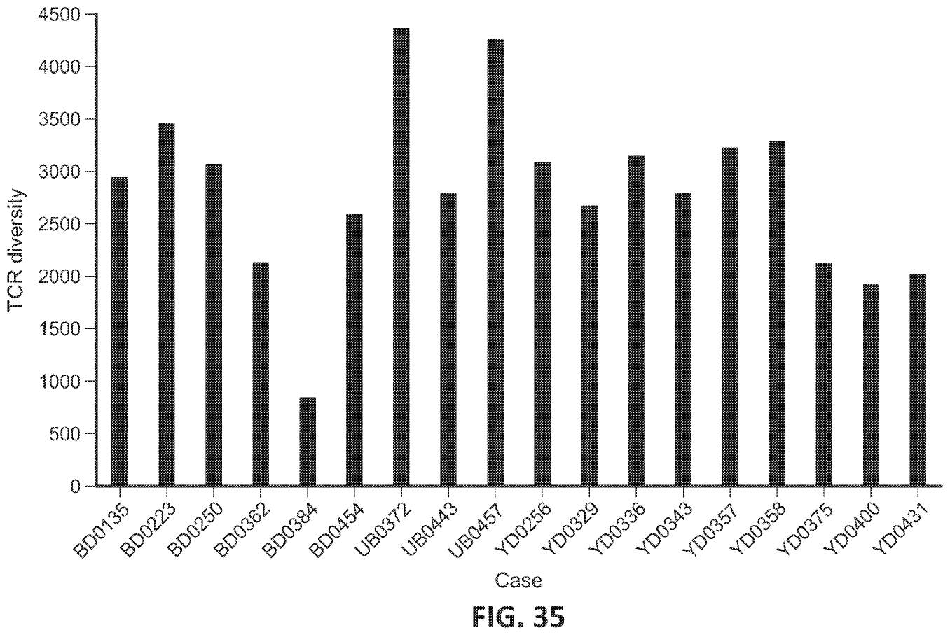

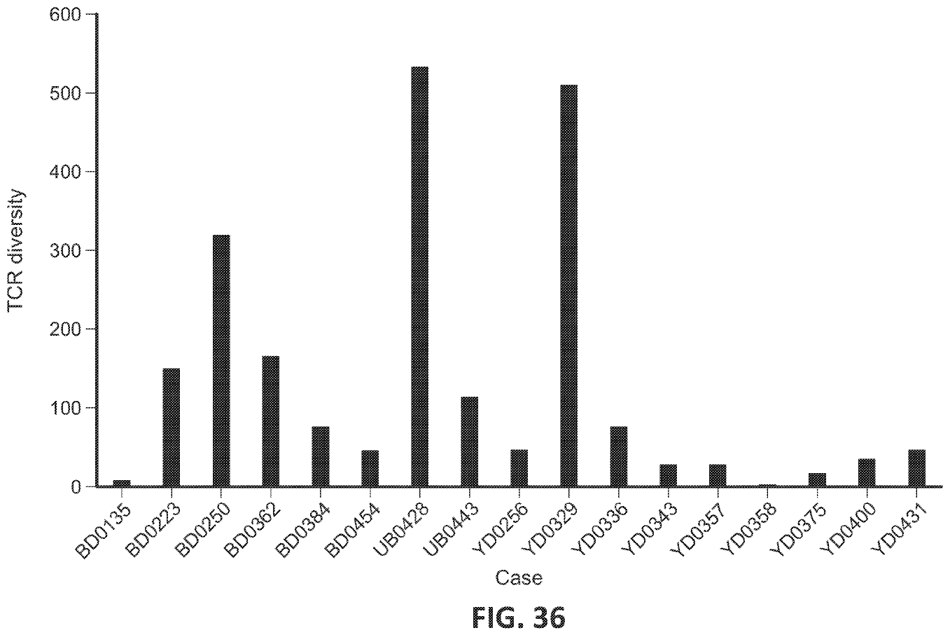

DNA was isolated from plasma, white blood cell, and tumor samples from patients with colorectal cancer. The methods described herein were used to analyze the TCR repertoires in each compartment. The results show the TCR diversity in plasma (FIG. 34), white blood cell (FIG. 35), and tumor (FIG. 36) samples.

The invention claimed is:

1. A method for determining a sequence of a double stranded DNA molecule of an immune cell receptor, the method comprising:
(a) attaching a 3′ adapter fragment to each 3′ end of the double-stranded DNA molecule and a 5′ adapter fragment to each 5′ end of the double-stranded DNA molecule to generate an adapted double-stranded DNA molecule, wherein the adapted double-stranded DNA molecule comprises an adapted Watson strand and an adapted Crick strand, wherein the 3′ adapter fragment comprises a molecular barcode, a primer sequence, and an adapter sequence, and wherein the molecular barcode of the adapted Watson strand is the reverse complement of the molecular barcode of the adapted Crick strand;
(b) copying both strands of the adapted double-stranded DNA molecule, wherein the copying comprises performing a round of linear extension of the adapted double-stranded DNA molecule, generating an adapted double-stranded Watson template and an adapted double-stranded Crick template;
(c) generating a first population of analyte DNA fragments from the adapted double-stranded Watson template and generating a first sequencing read for at least one member of the first population of analyte DNA fragments;
(d) generating a second population of analyte DNA fragments from the adapted double-stranded Crick template and generating a second sequencing read for at least one member of the second population of analyte DNA fragments;
(e) grouping the first sequencing reads according to the molecular barcode present on the at least one member of the first population of analyte DNA fragments to generate a first analyte DNA family;
(f) grouping the second sequencing reads according to the molecular barcode present on the at least one member of the second population of analyte DNA fragments to generate a second analyte DNA family;
(g) analyzing the first sequencing read of the first analyte DNA family; and
(h) analyzing the second sequencing read of the second analyte DNA family, thus, determining the sequence of the double stranded DNA molecule.

2. The method of claim 1, wherein the 3′ adaptor fragment comprises a partially double-stranded molecular barcode.

3. The method of claim 2, wherein the partially double-stranded molecular barcode comprises an endogenous barcode, an exogenous barcode, or both.

4. The method of claim 1, wherein the copying step (b) further comprises performing the round of linear extension of the adapted double-stranded DNA molecule with (i) a first primer complementary to the 3′ adapter sequence, and (ii) a second primer complementary to the complement of the 5′ adapter sequence.

5. The method of claim 1, wherein the generating steps (c) and (d) are performed under PCR conditions.

6. The method of claim 5, wherein the generating step (c) further comprises amplifying the adapted double-stranded Watson template with a first set of Watson-target selective primer pair, wherein the first set of Watson target-selective primer pair comprises (i) a first Watson target-selective primer comprising a sequence complementary to the 3′ adapter sequence, and (ii) a second Watson target-selective primer comprising a target-selective sequence.

7. The method of claim 6, wherein the second Watson target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65.

8. The method of claim 6, wherein the second Watson target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

9. The method of claim 5, wherein the generating step (d) further comprises amplifying the adapted double-stranded Crick template with a first set of Crick-target selective primer pair, wherein the first set of Crick target-selective primer pair comprises (i) a first Crick target-selective primer comprising a sequence complementary to the 3′ adapter sequence, and (ii) a second Crick target-selective primer comprising a target-selective sequence.

10. The method of claim 8, wherein the second Crick target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65.

11. The method of claim 8, wherein the second Crick target-selective primer comprises a sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26

12. The method of claim 1, wherein the double-stranded DNA molecule comprises a V(D)J sequence of the immune cell receptor.

13. The method of claim 12, wherein the target-selective sequence comprises a sequence complementary to the V(D)J sequence of the immune cell receptor.

14. The method of claim 1, wherein the immune cell receptor comprises a B cell receptor.

15. The method of claim 1, wherein the immune cell receptor comprises a T cell receptor.

16. The method of claim 1, further comprising identifying (i) a mutation in the adapted double-stranded Watson template of the first analyte DNA family, (ii) a mutation in the adapted double-stranded Crick template of the second analyte DNA family, or (iii) a mutation in both the adapted double-stranded Watson template and the adapted double-stranded Crick template.

17. The method of claim 16, wherein the mutation is selected from the group consisting of an insertion, a deletion, a substitution, a deletion-insertion, a duplication, an inversion, a frameshift, a repeat expansion, a translocation, and combinations thereof.

18. The method of claim 1, wherein the method determines the sequence of the double-stranded DNA molecule in a population of double-stranded DNA molecules by assaying both strands of the double-stranded DNA molecule.

19. The method of claim 18, wherein a mutation in both the adapted double-stranded Watson template and the adapted double-stranded Crick template is identified.


SEQUENCE LISTING (68 sequences)

SEQ ID NO: 1 - DNA, 59 bp, synthetic construct
  source 1..59: mol_type = other DNA
ataaaacgacggcnnnnnnnnnnnnnnagatcggaagagcacacgtctgaactccagtc

SEQ ID NO: 2 - skipped

SEQ ID NO: 3 - RNA, 14 nt, synthetic construct
  source 1..14: mol_type = other RNA
gccgtcgttttadt

SEQ ID NO: 4 - DNA, 19 bp, synthetic construct
  source 1..19: mol_type = other DNA
ctacaactgtgagtctggt

SEQ ID NO: 5 - DNA, 19 bp, synthetic construct
  source 1..19: mol_type = other DNA
gccttacctacaacggtta

SEQ ID NO: 6 - DNA, 19 bp, synthetic construct
  source 1..19: mol_type = other DNA
cctacaacagtgagccaac

SEQ ID NO: 7 - DNA, 17 bp, synthetic construct
  source 1..17: mol_type = other DNA
aagacagagagctgggt

SEQ ID NO: 8 - DNA, 19 bp, synthetic construct
  source 1..19: mol_type = other DNA
cctaggatggagagtcgag

SEQ ID NO: 9 - DNA, 17 bp, synthetic construct
  source 1..17: mol_type = other DNA
gtcacagtgagcctggt

SEQ ID NO: 10 - DNA, 17 bp, synthetic construct
  source 1..17: mol_type = other DNA
tacctagcacggtgagc

SEQ ID NO: 11 - DNA, 18 bp, synthetic construct
  source 1..18: mol_type = other DNA
cagtacggtcagcctaga

SEQ ID NO: 12 - DNA, 17 bp, synthetic construct
  source 1..17: mol_type = other DNA
taccgagcactgtcagc

SEQ ID NO: 13 - DNA, 18 bp, synthetic construct
  source 1..18: mol_type = other DNA
cttacccagcactgagag

SEQ ID NO: 14 - DNA, 16 bp, synthetic construct
  source 1..16: mol_type = other DNA
caccgagcaccaggag

SEQ ID NO: 15 - DNA, 16 bp, synthetic construct
  source 1..16: mol_type = other DNA
cacccagcacggtcag

SEQ ID NO: 16 - DNA, 16 bp, synthetic construct
  source 1..16: mol_type = other DNA
ctgtgaccgtgagcct

SEQ ID NO: 17 - DNA, 26 bp, synthetic construct
  source 1..26: mol_type = other DNA
cctgaaaaatgtcttacctacaactg

SEQ ID NO: 18 - DNA, 16 bp, synthetic construct
  source 1..16: mol_type = other DNA
acccccagccttacct

SEQ ID NO: 19 - DNA, 22 bp, synthetic construct
  source 1..22: mol_type = other DNA
gccttgacttactcacctacaa

SEQ ID NO: 20 - DNA, 25 bp, synthetic construct
  source 1..25: mol_type = other DNA
gaagtcttttacatacccaagacag

SEQ ID NO: 21 - DNA, 22 bp, synthetic construct
  source 1..22: mol_type = other DNA
ccctgattctgcaacttaccta

SEQ ID NO: 22 - DNA, 18 bp, synthetic construct
  source 1..18: mol_type = other DNA
cccccatacctgtcacag

SEQ ID NO: 23 - DNA, 19 bp, synthetic construct
  source 1..19: mol_type = other DNA
gagcccccttcttacctag

SEQ ID NO: 24 - DNA, 18 bp, synthetic construct
  source 1..18: mol_type = other DNA
gcctccttacccagtacg

SEQ ID NO: 25 - DNA, 16 bp, synthetic construct
  source 1..16: mol_type = other DNA
cccgcttaccgagcac

SEQ ID NO: 26 - DNA, 16 bp, synthetic construct
  source 1..16: mol_type = other DNA
ggccccagcttaccca

SEQ ID NO: 27 - DNA, 16 bp, synthetic construct
  source 1..16: mol_type = other DNA
gcccgcgctcaccgag

SEQ ID NO: 28 - DNA, 17 bp, synthetic construct
  source 1..17: mol_type = other DNA
ccgcgaaaactcaccca

SEQ ID NO: 29 - DNA, 17 bp, synthetic construct
  source 1..17: mol_type = other DNA
gcccgaatctcacctgt

SEQ ID NO: 30 - DNA, 22 bp, synthetic construct
  source 1..22: mol_type = other DNA
aatgtcttacctacaactgtga

SEQ ID NO: 31 - DNA, 18 bp, synthetic construct
  source 1..18: mol_type = other DNA
cccagccttacctacaac

SEQ ID NO: 32 - DNA, 24 bp, synthetic construct
  source 1..24: mol_type = other DNA
ccttgacttactcacctacaacag

SEQ ID NO: 33 - DNA, 21 bp, synthetic construct
  source 1..21: mol_type = other DNA
tacatacccaagacagagagc

SEQ ID NO: 34 - DNA, 22 bp, synthetic construct
  source 1..22: mol_type = other DNA
gcaacttacctaggatggagag

SEQ ID NO: 35 - DNA, 20 bp, synthetic construct
  source 1..20: mol_type = other DNA
ccatacctgtcacagtgagc

SEQ ID NO: 36 - DNA, 19 bp, synthetic construct
  source 1..19: mol_type = other DNA
cccttcttacctagcacgg

SEQ ID NO: 37 - DNA, 19 bp, synthetic construct
  source 1..19: mol_type = other DNA
cttacccagtacggtcagc

SEQ ID NO: 38 - DNA, 17 bp, synthetic construct
  source 1..17: mol_type = other DNA
ccgcttaccgagcactg

SEQ ID NO: 39 - DNA, 17 bp, synthetic construct
  source 1..17: mol_type = other DNA
ccagcttacccagcact

SEQ ID NO: 40 - DNA, 16 bp, synthetic construct
  source 1..16: mol_type = other DNA
cgctcaccgagcacca

SEQ ID NO: 41 - DNA, 18 bp, synthetic construct
  source 1..18: mol_type = other DNA
gaaaactcacccagcacg

SEQ ID NO: 42 - DNA, 19 bp, synthetic construct
  source 1..19: mol_type = other DNA
gaatctcacctgtgaccgt

SEQ ID NO: 43 - DNA, 25 bp, synthetic construct
  source 1..25: mol_type = other DNA
gcaaaagaacctgaaaaatgtctta

SEQ ID NO: 44 - DNA, 17 bp, synthetic construct
  source 1..17: mol_type = other DNA
ccctcctagagaccccc

SEQ ID NO: 45 - DNA, 21 bp, synthetic construct
  source 1..21: mol_type = other DNA
ctgtccagccttgacttactc

SEQ ID NO: 46 - DNA, 26 bp, synthetic construct
  source 1..26: mol_type = other DNA
ctatcccgaaagaagtcttttacata

SEQ ID NO: 47 - DNA, 20 bp, synthetic construct
  source 1..20: mol_type = other DNA
ccaccctgattctgcaactt

SEQ ID NO: 48 - DNA, 18 bp, synthetic construct
  source 1..18: mol_type = other DNA
aagagtggagcccccata

SEQ ID NO: 49 - DNA, 16 bp, synthetic construct
  source 1..16: mol_type = other DNA
ccacctggagccccct

SEQ ID NO: 50 - DNA, 17 bp, synthetic construct
  source 1..17: mol_type = other DNA
ccccaaccgcctcctta

SEQ ID NO: 51 - DNA, 16 bp, synthetic construct
  source 1..16: mol_type = other DNA
ttcagcgggagccccc

SEQ ID NO: 52 - DNA, 16 bp, synthetic construct
  source 1..16: mol_type = other DNA
cggcggccccagctta

SEQ ID NO: 53 - DNA, 16 bp, synthetic construct
  source 1..16: mol_type = other DNA
cagcagcccgcgctca

SEQ ID NO: 54 - DNA, 16 bp, synthetic construct
  source 1..16: mol_type = other DNA
gggtggtcccgcgaaa

SEQ ID NO: 55 - DNA, 18 bp, synthetic construct
  source 1..18: mol_type = other DNA
ggagacgcccgaatctca

SEQ ID NO: 56 - DNA, 25 bp, synthetic construct
  source 1..25: mol_type = other DNA
ctgaaaaatgtcttacctacaactg

SEQ ID NO: 57 - DNA, 17 bp, synthetic construct
  source 1..17: mol_type = other DNA
gacccccagccttacct

SEQ ID NO: 58 - DNA, 23 bp, synthetic construct
  source 1..23: mol_type = other DNA
ccttgacttactcacctacaaca

SEQ ID NO: 59 - DNA, 25 bp, synthetic construct
  source 1..25: mol_type = other DNA
gaagtcttttacatacccaagacag

SEQ ID NO: 60 - DNA, 24 bp, synthetic construct
  source 1..24: mol_type = other DNA
tgattctgcaacttacctaggatg

SEQ ID NO: 61 - DNA, 18 bp, synthetic construct
  source 1..18: mol_type = other DNA
cccccatacctgtcacag

SEQ ID NO: 62 - DNA, 19 bp, synthetic construct
  source 1..19: mol_type = other DNA
gagcccccttcttacctag

SEQ ID NO: 63 - DNA, 19 bp, synthetic construct
  source 1..19: mol_type = other DNA
gcctccttacccagtacgg

SEQ ID NO: 64 - DNA, 16 bp, synthetic construct
  source 1..16: mol_type = other DNA
gcccccgcttaccgag

SEQ ID NO: 65 - DNA, 17 bp, synthetic construct
  source 1..17: mol_type = other DNA
cccagcttacccagcac

SEQ ID NO: 66 - DNA, 16 bp, synthetic construct
  source 1..16: mol_type = other DNA
cgcgctcaccgagcac

SEQ ID NO: 67 - DNA, 17 bp, synthetic construct
  source 1..17: mol_type = other DNA
ccgcgaaaactcaccca

SEQ ID NO: 68 - DNA, 19 bp, synthetic construct
  source 1..19: mol_type = other DNA
cgaatctcacctgtgaccg